(19) United States
(12) Patent Application Publication
(10) Pub. No.: US 2026/0275419 A1
(43) Pub. Date: Sep. 17, 2026

(54) IMMUNE LANDSCAPE SIGNATURES FOR OCULAR INFLAMMATION

(71) Applicant: Adverum Biotechnologies, Inc., Redwood City, CA (US)

(72) Inventors: Ruslan Grishanin, Redwood City, CA (US); Brigit Riley, Redwood City, CA (US); Julian N. Ramos, Redwood City, CA (US)

(21) Appl. No.: 19/472,241

(22) PCT Filed: Apr. 5, 2024
(86) PCT No.: PCT/US2024/023438

§ 371 Date: Oct. 3, 2025

Related U.S. Application Data
(60) Provisional application No. 63/457,319, filed on Apr. 5, 2023.
(60) Provisional application No. 63/463,182, filed on May 1, 2023.
(60) Provisional application No. 63/466,160, filed on May 12, 2023.
(60) Provisional application No. 63/545,004, filed on Oct. 20, 2023.

(51) Int. Cl. C12Q 1/6883; A61K 9/00; A61K 31/573; A61K 45/06

(52) U.S. Cl. CPC C12Q 1/6883; A61K 9/0048; A61K 31/573; A61K 45/06; C12Q 2600/136; C12Q 2600/158

(57) ABSTRACT

The present disclosure relates to gene signatures of AAV-induced inflammatory response useful in gene therapy methods for the identification and treatment of individuals who are candidates to receive, or who have received an rAAV gene therapy treatment, and who have an increased likelihood of AAV-induced inflammation.

### CROSS REFERENCE TO RELATED APPLICATIONS

The present application claims priority to International Patent Application Number PCT/US2024/023438, which claims priority to US Provisional Patent Application Nos. 63/457,319, filed Apr. 5, 2023, 63/463,182, filed May 1, 2023, 63/466,160, filed May 12, 2023, and 63/545,004, filed Oct. 20, 2023, each of which is hereby incorporated by reference herein in its entirety for all purposes.

### FIELD

The present disclosure relates to the field of gene therapy, and particularly to gene signatures of AAV-induced inflammatory response useful in gene therapy methods for the identification and treatment of individuals who are candidates to receive, or who have received an rAAV gene therapy treatment, and who have an increased likelihood of AAV-induced inflammation.

### BACKGROUND

A promising approach to prevent and treat genetic and other acquired diseases and disorders is the delivery of therapeutic molecules with a gene therapy vector such as a viral vector. Adeno-associated virus (AAV) is a single-stranded DNA virus with a 4.7 kb genome. Recombinant AAV (rAAV) vectors have demonstrated the ability for efficient gene transfer to the target cells and tissues. These rAAV vectors have been widely investigated in preclinical and clinical studies due to their efficacy and safety profiles relative to other viral vectors, including retrovirus and lentivirus vectors. AAV-induced inflammation has been observed during rAAV gene therapy treatment in ocular and non-ocular tissues. The pre-existence of inflammation due to the disease, the route of administration of the rAAV and its efficacy of reaching target tissues, the serotype of rAAV capsid, and the vector dose can contribute to undesirable outcomes. One example is the inflammation and degeneration of dorsal root ganglia neurons in nonhuman primates (NHPs) following delivery of rAAVs to the central nervous system to treat neurological disorders.

There remains a need for optimized viral vectors that result in reduced occurrence of AAV-induced inflammation associated with gene therapy, such as ocular gene therapy. Such optimization requires an improved understanding of the mechanisms of AAV-induced inflammation and associated gene signatures, particularly immune gene pathway signatures. Such gene signatures can be used in methods to identify and treat patients at risk, track patient response to treatment with rAAV vectors, and carry out further studies to develop optimized viral vectors.

### SUMMARY

The present disclosure relates generally to the field of gene therapy and the cellular and physiological responses to treatment with rAAV vectors. In particular, the present disclosure is directed to immune pathway associated gene signatures that indicate an AAV-induced inflammatory response, and the use of these gene signatures in methods for identifying, treating, and monitoring individuals who are candidates to receive or have received an rAAV gene therapy treatment (e.g., an ocular rAAV treatment) and who are at risk of AAV-induced inflammation. The identified gene signatures include genes corresponding to cytokine profiles, cellular stressors, and innate and adaptive immunity induced by treatment with rAAV viral vectors, particularly intravitreal treatment, and the inflammatory responses that can occur in different regions of ocular tissues, such as the ciliary body, the iris, and/or the retina. The gene signatures can also be used in methods for screening rAAV vector compositions for decreased rAAV-induced inflammation. This summary is intended to introduce the subject matter of the present disclosure, but does not cover each and every embodiment, combination, or variation that is contemplated and described within the present disclosure. Further embodiments are contemplated and described by the disclosure of the detailed description, drawings, and claims.

In at least one embodiment, the present disclosure provides a method of selecting and treating a patient who has an increased likelihood of AAV-induced inflammation from a population of patients who are candidates for receiving rAAV treatment or who have received an rAAV treatment, the method comprising: (a) obtaining a biological sample from the patient who has received an rAAV treatment; (b) detecting expression levels of each of a set of genes in the biological sample; (c) determining whether the detected expression levels indicate an increased likelihood of AAV-induced inflammation by comparison to predetermined threshold expression levels for the set of genes; and (d) administering an anti-inflammatory agent to the patient if an increased likelihood of AAV-induced inflammation is determined.

In at least one embodiment, the present disclosure provides a method of identifying a patient in need of an anti-inflammatory treatment from a population of patients who have received an intravitreal rAAV therapy, the method comprising: (a) obtaining a biological sample from the patient who has received an rAAV treatment; (b) detecting expression levels of each of a set of genes in the biological sample; (c) determining whether the detected expression levels indicate an increased likelihood of AAV-induced inflammation by comparison to predetermined threshold expression levels for the set of genes; and (d) identifying the patient as in need of anti-inflammatory treatment if an increased likelihood of AAV-induced inflammation is determined.

In at least one embodiment, the present disclosure provides a method of screening a patient prior to intravitreal rAAV therapy to identify a patient at risk of AAV-induced inflammation, the method comprising: (a) obtaining a biological sample from the patient; (b) detecting expression levels of each of a set of genes in the biological sample; (c) determining whether the detected expression levels indicate an increased likelihood of AAV-induced inflammation by comparison to predetermined threshold expression levels for the set of genes; and (d) identifying the patient as in need of anti-inflammatory treatment if an increased likelihood of AAV-induced inflammation is determined.

In at least one embodiment, the present disclosure provides a method of providing immunosuppressive treatment in a patient who has received intravitreal rAAV therapy, the method comprising; (a) obtaining a biological sample from the patient who has received an rAAV treatment; (b) detecting expression levels of each of a set of genes in the biological sample; (c) determining whether the detected expression levels indicate an increased likelihood of AAV-induced inflammation by comparison to predetermined threshold expression levels for the set of genes; and (d) administering an immunosuppressive treatment to the patient if an increased likelihood of AAV-induced inflammation is determined.

In at least one embodiment, the present disclosure provides a method of screening an rAAV treatment for reduced AAV-induced inflammation comprising: (a) administering a dose of the rAAV treatment to a non-human subject; (b) obtaining a biological sample from the subject; (b) detecting expression levels of each of a set of genes in the biological sample; (d) comparing the detected expression levels to predetermined threshold expression levels of a control rAAV treatment.

In at least one embodiment of the methods of identifying, screening, and/or treating of the present disclosure, the biological sample comprises a first biological sample obtained at a first time point and a second biological sample obtained at a second time point.

In at least one embodiment of the methods of identifying, screening, and/or treating of the present disclosure, detecting the expression levels comprises sequencing RNA in the biological sample.

In at least one embodiment of the methods of identifying, screening, and/or treating of the present disclosure, the set of genes comprises at least 2, at least 3, at least 4, at least 5, at least 6, at least 7, at least 8, at least 9, at least 10, or more genes selected from A2M, B2M, BLNK, BTK, C1QA, C1QB, C1QC, C1R, C1S, C2, C3, C4B, CARD11, CASP8, CCL3, CCL4L2, CCL5, CD14, CD19, CD22, CD247, CD28, CD3D, CD3E, CD3G, CD4, CD40, CD40LG, CD72, CD74, CD79A, CD79B, CD86, CD8A, CD8B, CFHR4, CHUK, CIITA, CPB2, CR1, CR2, CTLA4, CTSS, CXCL10, CXCL11, CXCL8, CXCL9, DAPP1, F12, F13A1, F2RL3, FCGR1A, FCGR2A, FCGR3A, FYN, GRAP2, HCK, HLA-DMA, HLA-DMB, HLA-DOA, HLA-DOB, HLA-DPA1, HLA-DPB1, HLA-DQA1, HLA-DRA, HLA-DRB1, HLA-E, HLA-F, HLA-G, HSP90AA1, ICOS, IFI30, IFITIvII, IFITM1, IFNG, IKBKB, IKBKE, IKBKG, IL10, IL12A, IL12B, IL1B, IL6, INPP5D, IRF7, ITGAM, ITGAX, ITGB2, ITK, IY96, KLRC2, KLRD1, KPK3CD, LAT, LCK, LCP2, LGMN, LILRA1, LILRA4, LILRB2, LILRB4, LR2, LY97, MAP3K7, MAPK13, MP3K7, MYD88, NCF1, NFATC2, PAK5, PDCD1, PIK3AP1, PIK3CD, PLA2G4B, PLA2G4C, PLAU, PLCG2, PLD1, PRKCD, PROC, PSME1, PTPRC, RAC2, RASGRP3, SCIN, SERPINA1, SERPINA5, SERPINE1, STAT1, SYK, TAP1, TAP2, TLR1, TLR4, TLR7, TLR8, TNF, VASP, VAV1, VAV2, VSIG4, WAS, WAV1, and ZAP70.

In at least one embodiment of the methods of identifying, screening, and/or treating of the present disclosure, the set of genes comprises 30 or fewer, 25 or fewer, 20 or fewer, 15 or fewer, 10 or fewer, or 5 or fewer genes selected from A2M, B2M, BLNK, BTK, C1QA, C1QB, C1QC, C1R, C1S, C2, C3, C4B, CARD11, CASP8, CCL3, CCL4L2, CCL5, CD14, CD19, CD22, CD247, CD28, CD3D, CD3E, CD3G, CD4, CD40, CD40LG, CD72, CD74, CD79A, CD79B, CD86, CD8A, CD8B, CFHR4, CHUK, CIITA, CPB2, CR1, CR2, CTLA4, CTSS, CXCL10, CXCL11, CXCL8, CXCL9, DAPP1, F12, F13A1, F2RL3, FCGR1A, FCGR2A, FCGR3A, FYN, GRAP2, HCK, HLA-DMA, HLA-DMB, HLA-DOA, HLA-DOB, HLA-DPA1, HLA-DPB1, HLA-DQA1, HLA-DRA, HLA-DRB1, HLA-E, HLA-F, HLA-G, HSP90AA1, ICOS, IFI30, IFITIvII, IFITM1, IFNG, IKBKB, IKBKE, IKBKG, IL10, IL12A, IL12B, IL1B, IL6, INPP5D, IRF7, ITGAM, ITGAX, ITGB2, ITK, IY96, KLRC2, KLRD1, KPK3CD, LAT, LCK, LCP2, LGMN, LILRA1, LILRA4, LILRB2, LILRB4, LR2, LY97, MAP3K7, MAPK13, MP3K7, MYD88, NCF1, NFATC2, PAK5, PDCD1, PIK3AP1, PIK3CD, PLA2G4B, PLA2G4C, PLAU, PLCG2, PLD1, PRKCD, PROC, PSME1, PTPRC, RAC2, RASGRP3, SCIN, SERPINA1, SERPINA5, SERPINE1, STAT1, SYK, TAP1, TAP2, TLR1, TLR4, TLR7, TLR8, TNF, VASP, VAV1, VAV2, VSIG4, WAS, WAV1, and ZAP70.

In at least one embodiment, the set of genes is selected from:
(a) CXCL9, CXCL10, CXCL11, CCL5 and CCL3;
(b) CXCL9, CXCL11, CXCL10, IL6, TNF, CCL5, CXCL8, CCL3, TLR8, CCL4L2, IY96, IL12A, IL12B, STAT1, IKBKE, IL1B, TLR1, CASP8, IKBKG, TLR4, IRF7, CD14, MYD88, CD86, IKBKB, KPK3CD, MP3K7, CHUK, and CD40;
(c) CXCL10, CXCL9, CCL5, CCL4L2, and MYD88;
(d) CXCL10, CXCL9, CCL5, CXCL11, IL1B, TLR8, CCL4L2, CD86, TLR2, STAT1, TNF, LY97, TLR4, IRF7, TLR7 and IKBKE;
(e) PLA2G4C, PRKCG, RAC2, PTPRC, FCGR3A, FCGR2A, SYK, and FCGR1A;
(f) PLA2G4C, PTPRC, RAC2, LAT, NCF1, FCGR3A, WAV1, SYK, PLA2G4B, WAS, PLCG2, HCK, FCGR2A, INPP5D, FCGR2A, INPP5D, FCGR1A, PIK3CD, PRKCD, and VASP;
(g) RAC2, SYK, VAV2, FCGR3A and PTPRC;
(h) RAC2, FCGR2A, PTPRC, FCGR3A, SYK, VAV1, NCF1, PLA2G4C, WAS, SCIN, FCGR1A, INPP5D, HCK and PLD1;
(i) C1R, ITGAM, C4B, F13A1, ITGB2 and C1QC;
(j) SERPINA5, CR1, CR2, C4B, ITGAX, PROC, ITGAM, C3, F12, F13A1, ITGB2, SERPINE1, PLAU, C1S, C1QC, CFHR4, C1QA and C1R;
(k) ITGAX, FGG and CPB2;
(l) F13A1, ITGAX, C1QB, ITGAM, C1QC, C1QA, C2, ITGB2, C3, C4B, C1S, SERPINA1, VSIG4, SERPINA5, CR2, A2M, and F2RL3;
(m) HLA-DOB, KLRC2, CD8B, TAP1, HLA-DRA, CTSS, HLA-DMB, IFNG, CD8A and LGMN;
(n) HLA-DOB, KLRD1, TNF, IFNG, TAP1, HLA-G, CIITA, KLRC2, CD8B, TAP2, HLA-E, CD8A, CTSS, HLA-DPB1, HLA-DPA1, HLA-DRB1, HLA-DMB, HLA-DRA, HLA-DOA, HLA-F, CD74, HLA-DMA, IFI30, PSME1, HSP90AA1, and LGMN;
(o) HLA-DRB1, HLA-DQA1, HLA-DPA1, and KLRD1;
(p) HLA-DRA, HLA-G, B2M, HLA-DQA1, HLA-DRB1, HLA-DPB1, CIITA, CD74, HLA-DOA, HLA-DMB, HLA-DMA, HLA-E, CD8B, HLA-DPA1, CD4, TAP1, IFI30, KLRD1, HLA-DOB, TNF, CD8A, TAP2, and PSME1;
(q) CD79A, DAPP1, CD79B, CARD11, RAC2, BLNK, BTK, IFITIvII, and SYK;
(r) CD79A, CD19, CARD11, CD79B, DAPP1, CR2, LILRA4, CD22, RAC2, BTK, LILRA1, BLNK, IFITM1, LILRB2, LILRB4, VAV1, SYK, PIK3AP1, PLCG2, INPP5D, CD72, IKBKG, IKBKB, PIK3CD, and CHUK;
(s) DAPP1, RAC2, SYK, VAV2, and NFATC2; and
(t) RAC2, CD79A, CD79B, DAPP1, LILRA4, SYK, VAV1, BTK, BLNK, LILRB2, INPP5D, NFATC2, LILRB4, CD22, CARD11, CR2, LILRA1, CD72 and RASGRP3
(u) LCK, CD40LG, CD3D, CD28, CD3E, CARD11, PAK5, CD247, CD8B, ITK, PTPRC, LCP2, IFNG, CD8A, and FYN;
(v) LCK, CD3D, CARD11, CD28, TNF, ZAP70, CD40LG, CD3E, CD3G, PTPRC, ITK, IFNG, ICOS, PDCD1, LAT, LCP2, GRAP2, IL10, CD8B, CD8A, CD247, VAV1, PAK5, IKBKG, IKBKB, PIK3CD, MAPK13, MAP3K7, and CHUK;
(w) VAV2, CD40LG, CD28, PTPRC, and NFATC2; and
(x) CD3D, PTPRC, CD28, LCK, CD3G, VAV1, ICOS, CD8B, CD4, CD40LG, ITK, CD3E, TNF, CD8A, NFATC2, LCP2, CARD11, and CTLA4.

In at least one embodiment of the methods of identifying, screening, and/or treating of the present disclosure: (a) the AAV-induced inflammation is ocular inflammation; (b) the biological sample comprises ocular cells; (c) the biological sample comprises blood, serum, tears, or a combination thereof; (d) the anti-inflammatory agent is administered to the eye; (e) the rAAV treatment was administered to an eye of the subject; optionally, wherein the rAAV treatment was administered to the contralateral eye of the subject.

In at least one embodiment of the methods of identifying, screening, and/or treating of the present disclosure, the anti-inflammatory agent is a steroid. In at least one embodiment, the anti-inflammatory agent is a steroid and is administered systemically, administered orally, or administered topically. In at least one embodiment, the steroid is selected from corticosteroid, prednisone, difluprednate, and a combination thereof.

In at least one embodiment of the methods of identifying, screening, and/or treating of the present disclosure, the AAV-induced inflammation is in the ciliary body, in the iris, in the retina, and/or in the choroid.

In at least one embodiment of the methods of identifying, screening, and/or treating of the present disclosure, the AAV-induced inflammation is ocular inflammation, the expression levels of the set of genes in the biological sample relative to the predetermined threshold levels indicates a likelihood of AAV-induced inflammation in the ciliary body and/or the iris, and wherein the set of genes is selected from:
(a) CXCL9, CXCL10, CXCL11, CCL5 and CCL3;
(b) CXCL9, CXCL11, CXCL10, IL6, TNF, CCL5, CXCL8, CCL3, TLR8, CCL4L2, IY96, IL12A, IL12B, STAT1, IKBKE, IL1B, TLR1, CASP8, IKBKG, TLR4, IRF7, CD14, MYD88, CD86, IKBKB, KPK3CD, MP3K7, CHUK, and CD40;
(c) PLA2G4C, PRKCG, RAC2, PTPRC, FCGR3A, FCGR2A, SYK, and FCGR1A;
(d) PLA2G4C, PTPRC, RAC2, LAT, NCF1, FCGR3A, WAV1, SYK, PLA2G4B, WAS, PLCG2, HCK, FCGR2A, INPP5D, FCGR2A, INPP5D, FCGR1A, PIK3CD, PRKCD, and VASP;
(e) C1R, ITGAM, C4B, F13A1, ITGB2 and C1QC;
(f) SERPINA5, CR1, CR2, C4B, ITGAX, PROC, ITGAM, C3, F12, F13A1, ITGB2, SERPINE1, PLAU, C1S, C1QC, CFHR4, C1QA and C1R;
(g) HLA-DOB, KLRC2, CD8B, TAP1, HLA-DRA, CTSS, HLA-DMB, IFNG, CD8A and LGMN;
(h) HLA-DOB, KLRD1, TNF, IFNG, TAP1, HLA-G, CIITA, KLRC2, CD8B, TAP2, HLA-E, CD8A, CTSS, HLA-DPB1, HLA-DPA1, HLA-DRB1, HLA-DMB, HLA-DRA, HLA-DOA, HLA-F, CD74, HLA-DMA, IFI30, PSME1, HSP90AA1, and LGMN;
(i) CD79A, DAPP1, CD79B, CARD11, RAC2, BLNK, BTK, IFITIvII, and SYK; and
(j) CD79A, CD19, CARD11, CD79B, DAPP1, CR2, LILRA4, CD22, RAC2, BTK, LILRA1, BLNK, IFITM1, LILRB2, LILRB4, VAV1, SYK, PIK3AP1, PLCG2, INPP5D, CD72, IKBKG, IKBKB, PIK3CD, and CHUK;
(k) LCK, CD40LG, CD3D, CD28, CD3E, CARD11, PAK5, CD247, CD8B, ITK, PTPRC, LCP2, IFNG, CD8A, and FYN; and
(l) LCK, CD3D, CARD11, CD28, TNF, ZAP70, CD40LG, CD3E, CD3G, PTPRC, ITK, IFNG, ICOS, PDCD1, LAT, LCP2, GRAP2, IL10, CD8B, CD8A, CD247, VAV1, PAK5, IKBKG, IKBKB, PIK3CD, MAPK13, MAP3K7, and CHUK.

In at least one embodiment of the methods of identifying, screening, and/or treating of the present disclosure, the AAV-induced inflammation is ocular inflammation, the expression levels of the set of genes in the biological sample relative to the predetermined threshold levels indicates a likelihood AAV-induced inflammation in the retina, and wherein the set of genes is selected from:
(a) CXCL10, CXCL9, CCL5, CCL4L2, and MYD88;
(b) CXCL10, CXCL9, CCL5, CXCL11, IL1B, TLR8, CCL4L2, CD86, TLR2, STAT1, TNF, LY97, TLR4, IRF7, TLR7 and IKBKE;
(c) RAC2, SYK, VAV2, FCGR3A and PTPRC;
(d) RAC2, FCGR2A, PTPRC, FCGR3A, SYK, VAV1, NCF1, PLA2G4C, WAS, SCIN, FCGR1A, INPP5D, HCK and PLD1;
(e) ITGAX, FGG and CPB2;
(f) F13A1, ITGAX, C1QB, ITGAM, C1QC, C1QA, C2, ITGB2, C3, C4B, CIS, SERPINA1, VSIG4, SERPINA5, CR2, A2M, and F2RL3;
(g) HLA-DRB1, HLA-DQA1, HLA-DPA1, and KLRD1;
(h) HLA-DRA, HLA-G, B2M, HLA-DQA1, HLA-DRB1, HLA-DPB1, CIITA, CD74, HLA-DOA, HLA-DMB, HLA-DMA, HLA-E, CD8B, HLA-DPA1, CD4, TAP1, IFI30, KLRD1, HLA-DOB, TNF, CD8A, TAP2, and PSME1;
(i) DAPP1, RAC2, SYK, VAV2, and NFATC2; and
(j) RAC2, CD79A, CD79B, DAPP1, LILRA4, SYK, VAV1, BTK, BLNK, LILRB2, INPP5D, NFATC2, LILRB4, CD22, CARD11, CR2, LILRA1, CD72 and RASGRP3
(k) VAV2, CD40LG, CD28, PTPRC, and NFATC2; and
(l) CD3D, PTPRC, CD28, LCK, CD3G, VAV1, ICOS, CD8B, CD4, CD40LG, ITK, CD3E, TNF, CD8A, NFATC2, LCP2, CARD11, and CTLA4.

### INCORPORATION BY REFERENCE

The following passages describe different aspects of the invention in greater detail. Each aspect, embodiment, or feature of the invention may be combined with any other aspect, embodiment, or feature the invention unless clearly indicated to the contrary. All publications, patents, and patent applications mentioned in this specification are herein incorporated by reference in their entireties to the same extent as if each individual publication, patent, or patent application was specifically and individually indicated to be incorporated by reference.

### BRIEF DESCRIPTION OF THE DRAWINGS

A better understanding of the novel features and advantages of the present disclosure will be obtained by reference to the following detailed description that sets forth illustrative embodiments, in which the principles of the disclosure are utilized, and the accompanying drawings (also “Figure” and “FIG.” herein), of which:

FIG. 1 depicts the workflow for rAAV dose-response analysis in cynomolgus macaque NHPs to identify gene signatures as described in Example 1.

FIGS. 2A, 2B, 2C, and 2D depict results of a rAAV dose-response analysis of toll-like receptor signaling pathway gene expression changes in ciliary body-iris tissue at two dosages of IVT administered Ixo-vec as described in Example 1. FIG. 2A depicts a schematic diagram of the toll-like receptor signaling pathway in response to an Ixo-vec dose of 4E11 vg/eye. FIG. 2B plots the genes from this pathway that show significant log 2 fold-changes in expression between eyes dosed at 4E11 vg/eye and sham-injected eyes either individually (solid columns) or averaged together (box and whisker plot). FIG. 2C plots the genes from this pathway that show significant log 2 fold-changes in expression between eyes dosed at 2E13 vg/eye and sham-injected eyes either individually (solid columns) or averaged together (box and whisker plot). FIG. 2D depicts a schematic diagram of the toll-like receptor signaling pathway in response to an Ixo-vec dose of 2E13 vg/eye.

FIGS. 3A, 3B, 3C, and 3D depict results of a rAAV dose-response analysis of toll-like receptor signaling pathway gene expression changes in retina tissue at two dosages of IVT administered Ixo-vec as described in Example 1. FIG. 3A depicts a schematic diagram of the toll-like receptor signaling pathway in response to an Ixo-vec dose of 4E11 vg/eye. FIG. 3B plots the genes from this pathway that show significant log 2 fold-changes in expression between eyes dosed at 4E11 vg/eye and sham-injected eyes either individually (solid columns) or averaged together (box and whisker plot). FIG. 3C plots the genes from this pathway that show significant log 2 fold-changes in expression between eyes dosed at 2E13 vg/eye and sham-injected eyes either individually (solid columns) or averaged together (box and whisker plot).

FIG. 3D depicts a schematic diagram of the toll-like receptor signaling pathway in response to an Ixo-vec dose of 2E13 vg/eye.

FIGS. 4A, 4B, 4C, and 4D depict results of a rAAV dose-response analysis of FcγR-mediated phagocytosis pathway gene expression changes in ciliary body-iris tissue at two dosages of IVT administered Ixo-vec as described in Example 1. FIG. 4A depicts a schematic diagram of the FcγR-mediated phagocytosis pathway in response to an Ixo-vec dose of 4E11 vg/eye. FIG. 4B plots the genes from this pathway that show significant log 2 fold-changes in expression between eyes dosed at 4E11 vg/eye and sham-injected eyes either individually (solid columns) or averaged together (box and whisker plot). FIG. 4C plots the genes from this pathway that show significant log 2 fold-changes in expression between eyes dosed at 2E13 vg/eye and sham-injected eyes either individually (solid columns) or averaged together (box and whisker plot). FIG. 4D depicts a schematic diagram of the FcγR-mediated phagocytosis pathway in response to an Ixo-vec dose of 2E13 vg/eye.

FIGS. 5A, 5B, 5C, and 5D depict results of a rAAV dose-response analysis of FcγR-mediated phagocytosis gene expression changes in retina tissue at two dosages of IVT administered Ixo-vec as described in Example 1. FIG. 5A depicts a schematic diagram of the FcγR-mediated phagocytosis pathway in response to an Ixo-vec dose of 4E11 vg/eye. FIG. 5B plots the genes from this pathway that show significant log 2 fold-changes in expression between eyes dosed at 4E11 vg/eye and sham-injected eyes either individually (solid columns) or averaged together (box and whisker plot). FIG. 5C plots the genes from this pathway that show significant log 2 fold-changes in expression between eyes dosed at 2E13 vg/eye and sham-injected eyes either individually (solid columns) or averaged together (box and whisker plot).

FIG. 5D depicts a schematic diagram of the FcγR-mediated phagocytosis pathway in response to an Ixo-vec dose of 2E13 vg/eye.

FIGS. 6A, 6B, 6C, and 6D depict results of a rAAV dose-response analysis of complement and coagulation cascades pathway gene expression changes in ciliary body-iris tissue at two dosages of IVT administered Ixo-vec as described in Example 1. FIG. 6A depicts a schematic diagram of the complement and coagulation cascades pathway in response to an Ixo-vec dose of 4E11 vg/eye. FIG. 6B plots the genes from this pathway that show significant log 2 fold-changes in expression between eyes dosed at 4E11 vg/eye and sham-injected eyes either individually (solid columns) or averaged together (box and whisker plot). FIG. 6C plots the genes from this pathway that show significant log 2 fold-changes in expression between eyes dosed at 2E13 vg/eye and sham-injected eyes either individually (solid columns) or averaged together (box and whisker plot). FIG. 6D depicts a schematic diagram of the complement and coagulation cascades pathway in response to an Ixo-vec dose of 2E13 vg/eye.

FIGS. 7A, 7B, 7C, and 7D depict results of a rAAV dose-response analysis of complement and coagulation cascades gene expression changes in retina tissue at two dosages of IVT administered Ixo-vec as described in Example 1. FIG. 7A depicts a schematic diagram of the complement and coagulation cascades pathway in response to an Ixo-vec dose of 4E11 vg/eye. FIG. 7B plots the genes from this pathway that show significant log 2 fold-changes in expression between eyes dosed at 4E11 vg/eye and sham-injected eyes either individually (solid columns) or averaged together (box and whisker plot). FIG. 7C plots the genes from this pathway that show significant log 2 fold-changes in expression between eyes dosed at 2E13 vg/eye and sham-injected eyes either individually (solid columns) or averaged together (box and whisker plot). FIG. 7D depicts a schematic diagram of the complement and coagulation cascades pathway in response to an Ixo-vec dose of 2E13 vg/eye.

FIGS. 8A, 8B, 8C, and 8D depict results of a rAAV dose-response analysis of antigen processing and presentation pathway gene expression changes in ciliary body-iris tissue at two dosages of IVT administered Ixo-vec as described in Example 1. FIG. 8A depicts a schematic diagram of the antigen processing and presentation pathway in response to an Ixo-vec dose of 4E11 vg/eye. FIG. 8B plots the genes from this pathway that show significant log 2 fold-changes in expression between eyes dosed at 4E11 vg/eye and sham-injected eyes either individually (solid columns) or averaged together (box and whisker plot). FIG. 8C plots the genes from this pathway that show significant log 2 fold-changes in expression between eyes dosed at 2E13 vg/eye and sham-injected eyes either individually (solid columns) or averaged together (box and whisker plot). FIG. 8D depicts a schematic diagram of the antigen processing and presentation pathway in response to an Ixo-vec dose of 2E13 vg/eye.

FIGS. 9A, 9B, 9C, and 9D depict results of a rAAV dose-response analysis of antigen processing and presentation gene expression changes in retina tissue at two dosages of IVT administered Ixo-vec as described in Example 1. FIG. 9A depicts a schematic diagram of the antigen processing and presentation pathway in response to an Ixo-vec dose of 4E11 vg/eye.

FIG. 9B plots the genes from this pathway that show significant log 2 fold-changes in expression between eyes dosed at 4E11 vg/eye and sham-injected eyes either individually (solid columns) or averaged together (box and whisker plot). FIG. 9C plots the genes from this pathway that show significant log 2 fold-changes in expression between eyes dosed at 2E13 vg/eye and sham-injected eyes either individually (solid columns) or averaged together (box and whisker plot). FIG. 9D depicts a schematic diagram of the antigen processing and presentation pathway in response to an Ixo-vec dose of 2E13 vg/eye.

FIGS. 10A, 10B, 10C, and 10D depict results of a rAAV dose-response analysis of B cell receptor signaling pathway gene expression changes in ciliary body-iris tissue at two dosages of IVT administered Ixo-vec as described in Example 1. FIG. 10A depicts a schematic diagram of the B cell receptor signaling pathway in response to an Ixo-vec dose of 4E11 vg/eye.

FIG. 10B plots the genes from this pathway that show significant log 2 fold-changes in expression between eyes dosed at 4E11 vg/eye and sham-injected eyes either individually (solid columns) or averaged together (box and whisker plot). FIG. 10C plots the genes from this pathway that show significant log 2 fold-changes in expression between eyes dosed at 2E13 vg/eye and sham-injected eyes either individually (solid columns) or averaged together (box and whisker plot). FIG. 10D depicts a schematic diagram of the B cell receptor signaling pathway in response to an Ixo-vec dose of 2E13 vg/eye.

FIGS. 11A, 11B, 11C, and 11D depict results of a rAAV dose-response analysis of B cell receptor signaling pathway gene expression changes in retina tissue at two dosages of IVT administered Ixo-vec as described in Example 1. FIG. 11A depicts a schematic diagram of the B cell receptor signaling pathway in response to an Ixo-vec dose of 4E11 vg/eye. FIG. 11B plots the genes from this pathway that show significant log 2 fold-changes in expression between eyes dosed at 4E11 vg/eye and sham-injected eyes either individually (solid columns) or averaged together (box and whisker plot). FIG. 11C plots the genes from this pathway that show significant log 2 fold-changes in expression between eyes dosed at 2E13 vg/eye and sham-injected eyes either individually (solid columns) or averaged together (box and whisker plot).

FIG. 11D depicts a schematic diagram of the B cell receptor signaling pathway in response to an Ixo-vec dose of 2E13 vg/eye.

FIGS. 12A, 12B, and 12C depict results of a rAAV dose-response analysis of VEGF signaling pathway gene expression changes in ciliary body-iris tissue at two dosages of IVT administered Ixo-vec as described in Example 1. FIG. 12A depicts a schematic diagram of the VEGF signaling pathway in response to an Ixo-vec dose of 2E13 vg/eye. FIG. 12B plots the genes from this pathway that show significant log 2 fold-changes in expression between eyes dosed at 4E11 vg/eye and sham-injected eyes either individually (solid columns) or averaged together (box and whisker plot). FIG. 12C plots the genes from this pathway that show significant log 2 fold-changes in expression between eyes dosed at 2E13 vg/eye and sham-injected eyes either individually (solid columns) or averaged together (box and whisker plot).

FIGS. 12D, 12E, and 12F depict results of a rAAV dose-response analysis of VEGF signaling pathway gene expression changes in retina tissue at two dosages of IVT administered Ixo-vec as described in Example 1. FIG. 12D depicts a schematic diagram of the VEGF signaling pathway in response to an Ixo-vec dose of 2E13 vg/eye. FIG. 12E plots the genes from this pathway that show significant log 2 fold-changes in expression between eyes dosed at 4E11 vg/eye and sham-injected eyes either individually (solid columns) or averaged together (box and whisker plot). FIG. 12F plots the genes from this pathway that show significant log 2 fold-changes in expression between eyes dosed at 2E13 vg/eye and sham-injected eyes either individually (solid columns) or averaged together (box and whisker plot).

FIGS. 13A, 13B, 13C, and 13D depict results of a rAAV dose-response analysis of ciliary body fold formation and aqueous humor production occurring in pigmented ciliary epithelium (PCE) and nonpigmented ciliary epithelium (NPCE) pathway gene expression changes in ciliary body-iris tissue at three dosages of IVT administered Ixo-vec as described in Example 1. FIG. 13A depicts a schematic diagram of the pathway. Genes within the dashed rectangle have biological roles specific to pigmented ciliary epithelium (see e.g., Cicero 2009; Kramer 2013; Janssen 2012; Janssen 2014; Pang 2021). FIG. 13B plots the genes from this pathway that show significant log 2 fold-changes in expression between eyes dosed at 4E11 vg/eye and sham-injected eyes either individually (solid columns) or averaged together (box and whisker plot). FIG. 13C plots the genes from this pathway that show significant log 2 fold-changes in expression between eyes dosed at 2E12 vg/eye and sham-injected eyes either individually (solid columns) or averaged together (box and whisker plot). FIG. 13D plots the genes from this pathway that show significant log 2 fold-changes in expression between eyes dosed at 2E13 vg/eye and sham-injected eyes either individually (solid columns) or averaged together (box and whisker plot).

FIG. 14A shows a heatmap of composite scores for activation of differentially expressed genes (“DEGs”) from choroid tissue samples of the rAAV dose-response analysis of Example 1. The DEGs are associated with the specific biological process or with the regulation of the specific molecule indicated on the y-axis. The samples used in calculating the composite scores were as follows: G1 CHL indicates choroid from left (contralateral uninjected) eye of Group 1 animals (those receiving sham injection in right eye); G4 CHL indicates choroid from left (contralateral uninjected) eye of Group 4 animals (2E13 vg injected in right eye); G1 CHR indicates choroid from the right (sham injected) eye of Group 1 animals; G2 CHR indicates choroid from the right (4E11 vg injected) eye of Group 2 animals; and G4 CHR indicates choroid from the right (2E13 vg injected) eye of Group 4 animals.

FIG. 14B shows a heatmap of composite scores for activation of differentially expressed genes (“DEGs”) from retina tissue samples of the rAAV dose-response analysis of Example 1. The DEGs are associated with the specific biological process or with the regulation of the specific molecule indicated on the y-axis. The samples used in calculating the composite scores were as follows: G1 REL indicates retina from left (contralateral uninjected) eye of Group 1 animals (those receiving sham injection in right eye); G4 REL indicates retina from left (contralateral uninjected) eye of Group 4 animals (2E13 vg injected in right eye); G1 RER indicates retina from right (sham injected) eye of Group 1 animals; G2 RER indicates retina from right (4E11 vg injected) eye of Group 2 animals; G4 RER indicates retina from right (2E13 vg injected) eye of Group 4 animals.

FIG. 14C shows a heatmap of composite scores for activation of differentially expressed genes (“DEGs”) from ciliary body-iris tissue samples of the rAAV dose-response analysis of Example 1. The DEGs are associated with the specific biological process or with the regulation of the specific molecule indicated on the y-axis. The samples used in calculating the composite scores were as follows: G1 CIL indicates ciliary body-iris from left (contralateral uninjected) eye of Group 1 animals (those receiving sham injection in right eye); G3 CIL indicates ciliary body-iris from left (contralateral uninjected) eye of Group 3 animals (2E12 vg injected in right eye); G4 CIL indicates ciliary body-iris from left (contralateral uninjected) eye of Group 4 animals (2E13 vg injected in right eye); G1 CIR indicates ciliary body-iris from right (sham injected) eye of Group 1 animals; G2 CIR indicates ciliary body-iris from right (4E11 vg injected) eye of Group 2 animals; G3 CIR indicates ciliary body-iris from right (2E12 vg injected) eye of Group 3 animals; G4 CIR indicates ciliary body-iris from right (2E13 vg injected) eye of Group 4 animals.

FIG. 14D shows a heatmap of composite scores for activation of differentially expressed genes (“DEGs”) from spleen tissue samples of the rAAV dose-response analysis of Example 1. The DEGs are associated with the specific biological process or with the regulation of the specific molecule indicated on the y-axis. The samples used in calculating the composite scores were as follows: G1 SP indicates spleen from Group 1 (sham injected) animals; G3 SP indicates spleen from Group 3 (2E12 vg injected into right eye) animals; G4 SP indicates spleen from Group 4 (2E13 vg injected into right eye) animals.

FIGS. 15A, 15B, and 15C depict results of a rAAV dose-response analysis of endoplasmic reticulum stress pathway gene expression changes in ciliary body-iris tissue at two dosages of IVT administered Ixo-vec as described in Example 1. FIG. 15A depicts a schematic diagram of the pathway with darkened icons indicating the DEGs in ciliary body-iris tissue between sham-injected eyes and eyes dosed at 4E11 vg/eye. FIG. 15B depicts a schematic diagram of the pathway with darkened icons indicating the DEGs in ciliary body-iris tissue between sham-injected eyes and eyes dosed at 2E13 vg/eye. FIG. 15C plots the genes from this pathway that show significant log 2 fold-changes in expression between eyes dosed at 2E13 vg/eye and sham-injected eyes either individually (solid columns) or averaged together (box and whisker plot).

FIGS. 16A and 16B depict results of a rAAV dose-response analysis of endoplasmic reticulum stress pathway gene expression changes in retina tissue at two dosages of IVT administered Ixo-vec as described in Example 1. FIG. 16A depicts a schematic diagram of the pathway with no darkened icons indicating DEGs in ciliary body-iris tissue between sham-injected eyes and eyes dosed at 4E11 vg/eye. FIG. 16B depicts a schematic diagram of the pathway with darkened icons indicating the DEGs (CASP12 and XBP1) in ciliary body-iris tissue between sham-injected eyes and eyes dosed at 2E13 vg/eye.

FIGS. 17A and 17B depict results of a rAAV dose-response analysis of endoplasmic reticulum stress pathway gene expression changes in choroid tissue at two dosages of IVT administered Ixo-vec as described in Example 1. FIG. 17A depicts a schematic diagram of the pathway with no darkened icons indicating DEGs in ciliary body-iris tissue between sham-injected eyes and eyes dosed at 4E11 vg/eye. FIG. 17B depicts a schematic diagram of the pathway with no darkened icons indicating DEGs in choroid tissue between sham-injected eyes and eyes dosed at 2E13 vg/eye.

FIGS. 18A, 18B, 18C, and 18D show representative immunohistochemistry (IHC) staining for B lymphocyte markers in the anterior tissues of the eye. FIG. 18A shows no CD20+cell (left image) or CD138+ cells (right image) were observed in the vehicle (sham injected) eyes. FIG. 18B shows staining of CD20-expressing cells in eyes dosed at 4E11 vg/eye. FIG. 18C shows CD20-positive (left image) and plasma cell marker CD138-positive (right image) IHC staining in eyes dosed at 2E13 vg/eye. Positively stained cells are indicated by arrows. FIG. D shows CD20-positive cell clusters in anterior tissues of the eye at indicated Ixo-vec dose. Positively stained cells are indicated by arrows (FIG. 18B, 18C) or asterisks (FIG. 18D).

FIGS. 19A, 19B, and 19C show representative immunohistochemistry (IHC) staining for T lymphocyte markers in the anterior tissues of the eye. FIG. 19A shows that vehicle (sham injected) eyes had no detectable expression of either CD4+ (left image) or CD8+ T cells (right image). FIG. 19B shows staining of CD4-positive (left image) and CD8-positive (right image) T cells were observed at doses of 4E11 vg/eye. FIG. 19C shows staining of CD4-positive (left image) and CD8-positive (right image) T cells were observed at doses of 2E13 vg/eye. Positively stained cells are indicated by arrows.

FIG. 20 shows log 2 fold-changes in expression of genes that represent a portion of the genes associated with TLR signaling (top), complement activation (middle), and B cell receptor signaling (bottom) pathways in the ciliary body/iris tissues across dosed groups, relative to the sham-injected group. Cohort sizes are shown in the figure legend: 4E11 vg=2; 2E12 vg=7; 2E13vg=6.

FIG. 21 shows a heatmap of log 2 fold-changes in expression of genes associated with biological pathways and processes. Changes in gene expression were measured in ciliary body-iris tissue by RT-qPCR analysis and normalized to the sham-injected (vehicle) eyes.

FIG. 22 shows the levels of vitreous inflammation measured in non-human primates with indicated doses of Ixo-vec. Vitreous cell (VC) grading is reported as peak score using the Hackett-McDonald method.

FIG. 23 shows the levels of anterior chamber and vitreous chamber cell inflammation measured in non-human primates with the indicated doses of Ixo-vec. Anterior cell (AC) and vitreous cell (VC) grading is reported as peak score using the Hackett-McDonald method. The maximum score for each chamber is 4. For each eye, the scores of both AC and VC were summated. For each group, the cumulative AC and VC scores are reported as mean with standard deviation.

FIGS. 24A, 24B, 24C, and 24D depict results of a rAAV dose-response analysis of T cell receptor signaling pathway gene expression changes in ciliary body-iris tissue at two dosages of IVT administered Ixo-vec as described in Example 1. FIG. 24A depicts a schematic diagram of the T cell receptor signaling pathway in response to an Ixo-vec dose of 4E11 vg/eye.

FIG. 24B plots the genes from this pathway that show significant log 2 fold-changes in expression between eyes dosed at 4E11 vg/eye and sham-injected eyes either individually (solid columns) or averaged together (box and whisker plot). FIG. 24C depicts a schematic diagram of the T cell receptor signaling pathway in response to an Ixo-vec dose of 2E13 vg/eye. FIG. 24D plots the genes from this pathway that show significant log 2 fold-changes in expression between eyes dosed at 2E13 vg/eye and sham-injected eyes either individually (solid columns) or averaged together (box and whisker plot).

FIGS. 25A, 25B, 25C, and 25D depict results of a rAAV dose-response analysis of T cell receptor signaling pathway gene expression changes in retina tissue at two dosages of IVT administered Ixo-vec as described in Example 1. FIG. 25A depicts a schematic diagram of the T cell receptor signaling pathway in response to an Ixo-vec dose of 4E11 vg/eye. FIG. 25B plots the genes from this pathway that show significant log 2 fold-changes in expression between eyes dosed at 4E11 vg/eye and sham-injected eyes either individually (solid columns) or averaged together (box and whisker plot). FIG. 25C depicts a schematic diagram of the T cell receptor signaling pathway in response to an Ixo-vec dose of 2E13 vg/eye. FIG. 25D plots the genes from this pathway that show significant log 2 fold-changes in expression between eyes dosed at 2E13 vg/eye and sham-injected eyes either individually (solid columns) or averaged together (box and whisker plot).

FIGS. 26A and 26B show mRNA expression levels of TLR10. FIG. 26A depicts TLR10 expression changes in ciliary body-iris tissue at three dosages of IVT administered Ixo-vec as described in Example 1. Cohort sizes are indicated within the column of each group: 4E11 vg=2; 2E12 vg=7; 2E13 vg=6. FIG. 26B depicts TLR10 expression changes in blood at three dosages of IVT administered Ixo-vec and vehicle (sham IVT injection) control at multiple time points after dosage: Day 15, Week 7, and Week 13. Asterisks denote significant differences from vehicle group (* P<0.05; ** P<0.01).

FIGS. 27A and 27B show mRNA expression levels of CXCR3. FIG. 27A depicts CXCR3 expression changes in ciliary body-iris tissue at three dosages of IVT administered Ixo-vec as described in Example 1. Cohort sizes are indicated within the column of each group: 4E11 vg=2; 2E12 vg=7; 2E13 vg=6. FIG. 27B depicts CXCR3 expression changes in blood at three dosages of IVT administered Ixo-vec and vehicle (sham IVT injection) control at multiple time points after dosage: Day 15, Week 7, and Week 13. Asterisk denotes significant difference from vehicle group (* P<0.05).

### DETAILED DESCRIPTION

For the descriptions herein and the appended claims, the singular forms “a”, and “an” include plural referents unless the context clearly indicates otherwise. Thus, for example, reference to “a protein” includes more than one protein, and reference to “a compound” refers to more than one compound. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. The use of “comprise,” “comprises,” “comprising” “include,” “includes,” and “including” are interchangeable and not intended to be limiting. It is to be further understood that where descriptions of various embodiments use the term “comprising,” those skilled in the art would understand that in some specific instances, an embodiment can be alternatively described using language “consisting essentially of” or “consisting of.”

Where a range of numeric values is provided, unless the context clearly dictates otherwise, it is understood that each intervening integer of the value, and each tenth of each intervening integer of the value, unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range.

Numeric ranges are inclusive of the numbers defining the range. The term about is used herein to mean plus or minus ten percent (10%) of a value. For example, “about 100” refers to any number between 90 and 110. The term “about” a range refers to that range minus 10% of its lowest value and plus 10% of its greatest value. Reference to “about” a value or parameter herein includes (and describes) embodiments that are directed to that value or parameter per se.

A scientific format of exponential notation may be used in the present disclosure where part of a number is replaced with En, in which E (exponent) multiplies the preceding number by 10 to the nth power. For example, a 2-decimal scientific format displays 12345678901 as 1.23E10, which is 1.23 times 10 to the 10th power, and may be written alternatively as 1.23E+ 10 or 1.23×10¹⁰.

Generally, the nomenclature used herein and the techniques and procedures described herein include those that are well understood and commonly employed by those of ordinary skill in the art, such as the common techniques and methodologies described in e.g., Green and Sambrook, Molecular Cloning: A Laboratory Manual (Fourth Edition), Vols. 1-3, Cold Spring Harbor Laboratory, Cold Spring Harbor, N.Y., 2012 (hereinafter “Sambrook”); and Current Protocols in Molecular Biology, F. M. Ausubel et al., eds., originally published in 1987 in book form by Greene Publishing Associates, Inc. and John Wiley & Sons, Inc., and regularly supplemented through 2011, and now available in journal format online as Current Protocols in Molecular Biology, Vols. 00-130, (1987-2020), published by Wiley & Sons, Inc. in the Wiley Online Library (hereinafter “Ausubel”).

Unless otherwise indicated, nucleic acids are written left to right in 5′ to 3′ orientation; amino acid sequences are written left to right in amino to carboxy orientation, respectively.

Unless defined otherwise, all technical and scientific terms used herein have the same meaning as commonly understood by one of ordinary skill in the art to which the present invention pertains. It is to be understood that the terminology used herein is for describing particular embodiments only and is not intended to be limiting. For purposes of interpreting this disclosure, the following description of terms will apply and, where appropriate, a term used in the singular form will also include the plural form and vice versa.

The headings provided herein are not limitations of the various aspects or embodiments of the invention which can be had by reference to the specification as a whole. Accordingly, the terms defined immediately below are more fully defined by reference to the specification as a whole.

The terminology used herein is for the purpose of describing particular examples only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”. The term “comprising” as used herein is synonymous with “including” or “containing,” and is inclusive or open-ended.

An “AAV vector” or “rAAV vector” as used herein refers to an adeno-associated virus (AAV) vector or a recombinant AAV (rAAV) vector comprising a polynucleotide sequence not of AAV origin (e.g., a polynucleotide heterologous to AAV such as a nucleic acid sequence that encodes a therapeutic transgene, e.g., human complement factor inhibitor (CFI) for transduction into a target cell or to a target tissue. In general, the heterologous polynucleotide is flanked generally by two AAV inverted terminal repeat sequences (ITRs). The term rAAV vector encompasses both rAAV vector particles and rAAV vector plasmids. A rAAV vector may be either single-stranded (ssAAV) or self-complementary (scAAV).

An “AAV virus” or “AAV viral particle” or “rAAV vector particle” or “rAAV particle” refers to a viral particle comprising at least one AAV capsid protein and a polynucleotide rAAV vector. In some cases, the AAV capsid protein is from a wild type AAV or is a variant AAV capsid protein. By “variant AAV capsid protein” is intended that the AAV capsid protein comprises at least one amino acid difference (e.g., amino acid substitution, amino acid insertion, amino acid deletion) relative to a corresponding parental AAV capsid protein. The variant capsid protein may confer increased infectivity of a retinal cell as compared to the infectivity of a retinal cell by an AAV virion comprising an amino acid sequence present in a naturally occurring AAV capsid protein. Variant AAV capsid proteins may include, but are not limited to, an AAV capsid protein with an insertion, an insertion of the 7m8 amino sequence, an R100 insertion, a 7m8 like insertion, an LSV1 sequence replacement and any other engineered capsid protein generated by other strategies (e.g., DNA shuffling, directed evolution, peptide insertion, ancestral reconstruction, among others). The LSV1 replacement sequence and the 7m8 insertion sequence are known in the art (see, for example, U.S. Pat. Nos. 9,193,956; 9,233,133; U.S. Pub. No. US2021/0040501; and PCT/US2020/029895). Variant AAVs of particular interest may include, but are not limited to, those disclosed in U.S. Pat. No. 9,193,956. In some embodiments, the variant AAV comprises or consists of the 7m8 variant capsid protein (which may be referred to as AAV2.7m8 and 7m8AAV2). In some embodiments, the AAV comprises or consists of an AAV2.5T capsid protein such as provided in U.S. Pat. No. 9,233,131. In some embodiments, the AAV comprises the AAVShH10 or AAV6 capsid protein (U.S. Patent Application Pub. No. 20120164106 and Klimczak et al PLOS One 4(10):e7467 (Oct. 14, 2009). In some embodiments, the AAV comprises or consists of an AAV2.5T_LSV1 variant disclosed in U.S. Patent App Pub. No. WO2020219933.

The terms “gene of interest”, “GOI” and “transgene” are used interchangeably herein. A gene of interest comprises an open reading frame encoding a gene product of interest. In various aspects, a nucleic acid may comprise two or more nucleic acid sequences each comprising a gene of interest encoding a gene product. A “gene product” is a molecule resulting from expression of a particular gene. Gene products include, but are not limited to, a polypeptide, an aptamer, an interfering RNA, an mRNA, and the like. In particular embodiments, a “gene product” is a polypeptide, peptide, protein or interfering RNA including short interfering RNA (siRNA), miRNA or small, hairpin RNA (shRNA). In some embodiments, the gene of interest may be a reporter gene. Reporter genes are known in the art and include, but are not limited to, chloramphenicol acetyl transferase, a P-galactosidase, a β-glucuronidase, a renilla luciferase, a firefly luciferase, a green fluorescent protein (GFP), a red fluorescent protein (RFP) and an alkaline phosphatase such as secreted alkaline phosphatase. In some embodiments, the gene product is a secreted or a transmembrane protein or polypeptide. In some embodiments, the gene of interest may encode one or more AAV proteins or polypeptides. AAV proteins or polypeptides may include, but are not limited to Rep proteins, Rep78, Rep 68, Rep58, Rep40, Cap proteins, VP1, VP2, VP3 and fragments and variants thereof, including but not limited to Rep/Cap. In some embodiments, the gene of interest may encode a reporter gene product such as, but not limited to, GFP and RFP.

In some embodiments, the gene of interest may encode a therapeutic gene product such as a therapeutic protein. Therapeutic gene products are known in the art and include, but are not limited to, a polypeptide hormone, cytokine or growth factor (e.g. insulin or erythropoietin), an interferon, a blood clotting factor, a vaccine, an anti-angiogenic polypeptide, a vascular endothelial growth factor (VEGF) binding protein, an anti-VEGF agent, an anti-VEGF protein, an opsin protein, an anti-C3 antibody, an anti-C5 antibody, a hormone receptor (such as but not limited to mineral corticosteroid, glucocorticoid, and thyroid hormone receptors), intramembrane proteins (such as but not limited to TM-1 and TM-7), intracellular receptors (such as but not limited to orphans, retinoids, vitamin D3 and vitamin A receptors), signaling molecules (such as but not limited to kinases, transcription factors and signal transducers and activators of transcription receptors of the cytokine superfamily (e.g. erythropoietin, growth hormone, interferons, interleukins and colony stimulating factors); G-protein coupled receptors such as but not limited to hormones, calcitonin, epinephrine, gastrin, paracrine or autocrine mediators such as somatostatin or prostaglandins; neurotransmitter receptors (norepinephrine, dopamine, serotonin or acetylcholine); ligands of tyrosine kinase receptors such as insulin growth factor and nerve growth factor; and an anti-dry AMD gene product. Anti-VEGF agents are known in the art and include, but are not limited to, bevacizumab, brolucizumab, ranibizumab, faricimab, abicipar pegol, conbercept, OPT-302, KSI-301, injectable sunitinib maleate (GB-102), PAN-90806 (PanOptica), and/or aflibercept.

Therapeutic gene products for use in treating an ocular disease or disorder may include, but are not limited to, an anti-angiogenic polypeptide, a VEGF binding protein, an opsin protein, an anti-C3 antibody, an anti-C5 antibody, an anti-dry AMD gene product, sFLT-1, CFI, or an anti-VEGF agent known in the art, including, but not limited to, bevacizumab, brolucizumab, ranibizumab, faricimab, abicipar pegol, conbercept, OPT-302, KSI-301, injectable sunitinib maleate (GB-102), PAN-90806 (PanOptica), and/or aflibercept. An anti-dry AMD gene product may include, but is not limited to, inhibitors of C3, C5, HtrA1, C1qm, and natural inhibitors of the complement pathway, such as CFI, CFH and CD59.

In at least one embodiment, the methods of the present disclosure can be used treating ocular diseases or disorders is selected from the group comprising glaucoma, retinitis pigmentosa, macular degeneration, retinoschisis, Leber's Congenital Amaurosis, diabetic retinopathy, achromotopsia and color blindness. In some aspects, the macular degeneration is selected from the group comprising dry macular degeneration, wet macular degeneration and age-related macular degeneration. In some aspects of the methods, the therapeutic gene product is selected from the group comprising an anti-angiogenic polypeptide, a vascular endothelial growth factor (VEGF) binding protein, an opsin protein, an anti-C3 antibody, an anti-C5 antibody, complement factor I (CFI) and an anti-dry AMD gene product. In certain aspects of the methods, the therapeutic gene product is selected from the group comprising an anti-angiogenic polypeptide, a VEGF binding protein, an opsin protein, an anti-C3 antibody, an anti-C5 antibody, an anti-dry AMD gene product, sFLT-1, CFI, or an anti-VEGF agent known in the art, including, but not limited to, bevacizumab, brolucizumab, ranibizumab, faricimab, abicipar pegol, conbercept, OPT-302, KSI-301, injectable sunitinib maleate (GB-102), PAN-90806 (PanOptica), and/or aflibercept.

The term “gene” as used herein refers to a nucleic acid molecule comprising a sequence encoding a polypeptide, and optionally, comprising sequences that direct and/or control expression of the coding sequence. Genes include genes included in the signatures identified herein as well as variants of those genes. The sequences of the genes listed herein can readily be obtained by one of skill in the art from publicly available databases, such as but not limited to the GenBank database maintained by the National Center for Biotechnology (NCBI), for example, by searching using the provided gene symbols. These gene symbols are recognized by databases including but not limited to HGNC, Entrez Gene, UniProtKB/Swiss-Prot, OMIM, GeneLoc, and/or Ensembl; all aliases listed herein are defined by the GeneCards database.

The terms “gene signature” or “gene expression profile” as used herein, refer to a set of genes for which the expression levels when taken together are indicative and/or predictive of a certain condition, such as inflammation. The term “differential expression” as used herein refers to the quantitative and/or qualitative differences in the expression levels of one or more genes in sample (e.g., a sample of rAAV-treated ocular cells) relative to another sample (e.g., a sample of non-treated ocular cells). A gene is determined to be differentially expressed when the measured level of its expression is determined to be either higher or lower by some predetermined threshold level in a sample relative to the level of its expression in a control sample. In at least one embodiment, the threshold for differential expression is set as a log 2 fold-change in expression of greater than 10.61.

A range of methods for determining expression levels of genes are well-known in the art. Generally, it is contemplated that any method of determining expression levels of genes may be used in the methods of the application. Methods of determining expression level include, but are not limited to, methods comprising detecting nucleic acids (e.g., RNA transcripts or cDNA) encoded by each of the plurality of genes in a gene signature. A range of technique are known in the art and can be used in the methods of the present disclosure, including but not limited to RNA sequencing based methods (e.g., RNA-seq), DNA sequencing based methods, PCR-based methods, reverse transcriptase-(RT) PCR methods, Q-beta replicase amplification, ligase chain reaction, signal amplification (Ampliprobe), light cycling, differential display, Northern analysis, hybridization, microarray analysis, MassArray analysis and MALDI-TOF mass spectrometry.

Methods of determining expression levels based on detecting protein molecules (e.g., immunoassays, or histological assays) may also be used in the methods of the application. Immunoassay methods for measuring the presence and quantity of a protein in a biological or cell sample are known in the art. See, for example, Hage, D. S. (1999) “Immunoassays” Analytica Chemistry 71(12):294-304; The Immunoassay Handbook, 4^{th} Edition: Theory and Applications of Ligand Binding, ELISA and Related Techniques by David Wild (Ed) Elsevier Science (2013). Immunoassays are generally based on the reaction between a target protein and an antibody or antibody fragment specifically binding to the target protein. Immunoassay may be performed in a liquid or solid phase. Suitable immunoassays include, but are not limited to, sandwich and competition assays, Western blotting, ELISAs, radioimmunoassays, fluoroimmunoassays and the like. The biological sample can be a cell culture medium or supernatant (a sample take from the culture without lysing the cells), cell lysate, whole cells (e.g., peripheral blood mononuclear cells (PBMCs)), blood (sometimes referred to as whole blood), serum, plasma, aqueous humor, vitreous humor or other body fluid or tissue. It is recognized that a biological sample from a subject may be enriched by separation of whole cells from the sample, particularly when the polypeptide of interest may be secreted from a cell. Separation may be by any convenient separation technique known in the art including, but not limited to, fluorescence activated cell sorting (FACS), magnetic separation, affinity chromatography, “panning” with an affinity reagent, centrifugation and ultracentrifugation.

The term “subject”, “patient”, or “individual” refers to a mammal including but not limited to, primates, such as humans and non-human primates (“NHPs”), e.g., African green monkeys and rhesus monkeys, mammalian sport animals, mammalian farm animals, mammalian pets and rodents. In some embodiments, the subject is a human.

Biological samples useful in the methods of the present disclosure include any sample obtained from a subject contains the biological molecules (e.g., RNA or DNA) relevant to the method for measuring the expression levels of the gene signature that identifies the risk of inflammatory response. Accordingly, it is contemplated that biological samples can comprise blood, intravitreal fluid, cerebrospinal fluid, peritoneal fluid, pleural fluid, bronchoalveolar lavage fluid, sputum, nasal brushings, throat swabs, urine, amniotic fluid, plasma, serum, saliva, semen, bone marrow, tissue or fine needle biopsy samples, stool, skin, or cells therefrom. In another embodiment, the biological sample comprises blood. The biological sample can be obtained from the subject at any point relevant to the particular method, such as prior to any rAAV treatment (e.g., screening potential candidates), or any time during or after rAAV treatment (e.g., monitoring patients). It is also contemplated that the biological samples can include tissue sections, such as frozen sections for immuno-histological analysis.

The terms “treat,” “treating”, “treatment,” and other grammatical equivalents as used herein, refer to alleviating, abating or ameliorating a disease or disorder, or symptoms of a disease or disorder, preventing additional symptoms of the disease or disorder, ameliorating or preventing the underlying causes of symptoms, inhibiting a disease or disorder, e.g., arresting the development of a disease or disorder, relieving a disease or disorder, causing regression of a disease or disorder, or stopping the symptoms of a disease or disorder, and are intended to include prophylaxis and prevention. The terms further include achieving a therapeutic benefit and/or a prophylactic benefit, such as the benefit achieved with the eradication or amelioration of one or more of the physiological symptoms associated with a disease or disorder such that an improvement is observed in the subject, notwithstanding that, the subject may still be afflicted with a disease or disorder.

The terms “administer,” “administering”, “administration,” and the like, as used herein, can refer to the methods that are used to enable delivery of therapeutics or pharmaceutical compositions to the desired site of biological action. These methods include intravitreal or subretinal injection to an eye.

The term “pharmaceutical composition,” or simply “composition” as used herein, can refer to a biologically active compound, optionally mixed with at least one pharmaceutically acceptable chemical component, such as, though not limited to carriers, stabilizers, diluents, dispersing agents, suspending agents, thickening agents, excipients and the like.

The terms “effective amount”, “therapeutically effective amount” or “pharmaceutically effective amount” as used herein, can refer to a sufficient amount of at least one pharmaceutical composition or compound being administered which will relieve to some extent one or more signs or symptoms of the ocular disease, ocular disorder or ocular condition being treated. In some embodiment, the effective amount of a pharmaceutical composition may be administered to a subject in need thereof as a unit dose (as described in further detail elsewhere herein).

Gene Signatures of Increased rAAV-Induced Inflammation Risk

rAAV-induced inflammation is a side-effect experienced by many patients receiving treatment with rAAV gene therapy. The inflammatory response induced by rAAV treatment, however, is not well understood and the molecular level. Proposed mechanisms responsible for rAAV-induced inflammation include toxicity related to overproduction of exogenous protein, and general immuno pathways previously identified in gene therapy studies.

The present disclosure is directed to methods for identifying immuno-related pathways and associated gene signatures useful in methods for identifying and mitigating an increased risk of an rAAV-induced inflammatory response in a patient. For example, the gene signatures can be used in methods for selecting, treating, and monitoring subjects who are candidates to receive an rAAV gene therapy, or who are receiving an rAAV gene therapy during their course of treatment. These methods can thereby help optimize the risk-benefit profile of any rAAV treatment used in humans.

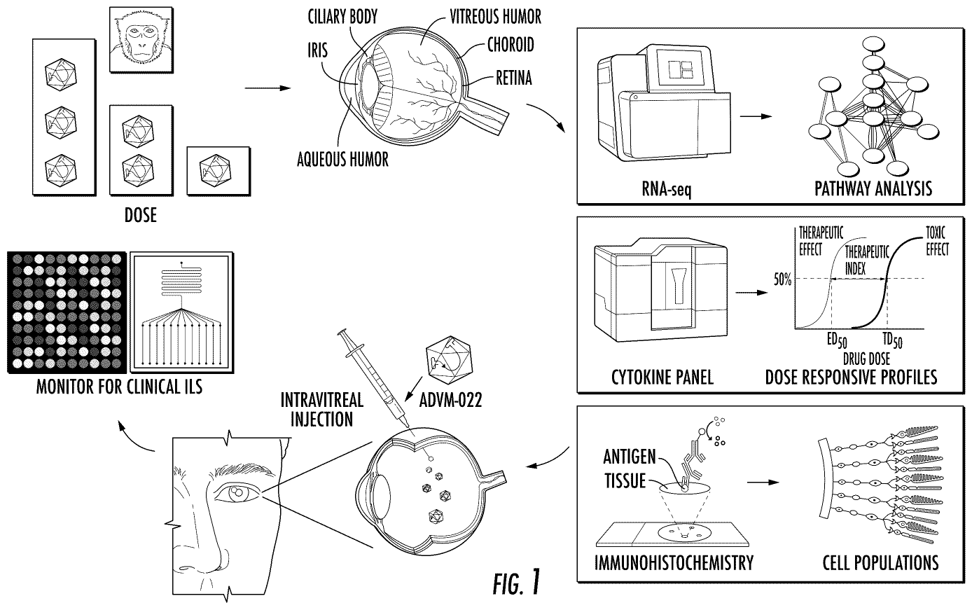

FIG. 1 depicts the workflow in a study of an exemplary rAAV gene therapy, Ixo-vec, used in dose response treatment in NHPs for identification the pathways and associated gene signatures useful for determining increased risk of rAAV-induced inflammatory response.

Ixo-vec (also known as, Ixoberogene soroparvovec or ADVM-022) is an adeno-associated virus (AAV) gene therapy vector encoding aflibercept packaged in the chimeric AAV.7m8 capsid. Ixo-vec turns ocular cells into biofactories that express and secrete the anti-VEGF drug, aflibercept. Ixo-vec is currently undergoing clinical trials for the treatment of ocular diseases mediated by abnormal production of VEGF, such as wet age-related macular degeneration (wAMD). To date, a single IVT injection of ADVM-022 has resulted in robust levels of aflibercept at therapeutic concentrations out as long as 104 weeks, as demonstrated in an ongoing, long-term clinical study, OPTIC. ADVM-022 has been shown to reduce annual injection frequency by >80%, and in most patients eliminate the need for any additional injections of anti-VEGF. Previous non-clinical studies demonstrated a nearly flat dose response suggesting a lower dose could also yield efficacious aflibercept levels.

In phase 1 OPTIC trial patients, ADVM-022 treatment has been demonstrated to induce long-term anti-VEGF expression of at least 3 years. In the OPTIC study, dose dependent mild to moderate inflammation was seen at doses of 2E11 vg/eye and 6E11 vg/eye. This inflammation was found responsive to topical corticosteroid therapy. Currently, a 2E11 dose of ADVM-022, as well as a lower 6E10 vg/eye dose are being evaluated in a phase 2 clinical study to expand the therapeutic window in humans.

Data from NHP studies of ADVM-022 have demonstrated intravitreal (IVT) administration results in a non-dose proportional aflibercept levels across several orders of magnitude of dosing. Levels at 3E10 vg/eye (human equivalent dose [HED](6E10)) were nearly equivalent to aflibercept levels observed at higher doses (up to 2E13 vg/eye). NHP data has also demonstrated that inflammation is dose-dependent, with little to no inflammation (e.g., Hackett-McDonald vitreous cell scores of 0 to 0.5) observed at the 3E10 and IE11 vg/eye doses (HED 6E10 and 2E11) to inflammation requiring corticosteroids at 2E12 vg/eye (HED 4E12 vg/eye) or greater.

As illustrated in FIG. 1, increasing doses of rAAV vector expressing Ixo-vec are administered in a single eye to cynomolgus macaques by intravitreal injection (top left, FIG. 1). Some time period after administration (e.g., 3 months) after rAAV dose, animals are euthanized and biological sample comprising ocular tissues and liquid are isolated (top middle, FIG. 1). Aqueous humor and vitreous humor from the samples are allocated for cytokine panel or measurement of the transgene protein (e.g., aflibercept). Tissues corresponding to multiple regions of ocular anatomy can be processed for immunohistochemistry or dissected further to isolate anatomical regions (choroid, retina, ciliary body/iris) for subsequent RNA-seq analysis (right panels). RNA-seq analysis provides expression level changes of genes in each of the samples at various dosages and over time. The RNA expression level changes can be analyzed to identify pathways that are significantly modulated at each dose of the rAAV treatment in comparison to sham-injected controls. Aqueous and vitreous humor analyses evaluate dose responsive RNA expression profiles. Parallel immunohistochemistry and histology, guided by the RNA expression analysis, can be used to characterize and quantify further the relevant immune cell populations and provide support for identifying optimal gene signatures. The resulting immune response gene signatures can be used to predict likelihood of AAV-induced inflammation in ocular tissues and guide patient dosing of rAAV vector (bottom center, FIG. 1).

Further, as exemplified by the RNA-seq analysis results described in the Examples, distinct expression profiles of cytokines, cellular stressors, and innate and adaptive immunity, induced by treatment with rAAV viral vectors, can result in different inflammatory responses in different regions of ocular tissues, such as the ciliary body, the iris, and/or the retina.

Genes exhibiting differential expression were identified in the following immuno-related pathways: toll-like receptor signaling pathway; FcγR mediated phagocytosis pathway; complement and coagulation cascades pathway; antigen processing and presentation pathway; B cell receptor signaling pathway; VEGF signaling pathway; aqueous humor production and ciliary body fold formation pathway; and/or the endoplasmic reticulum stress pathway.

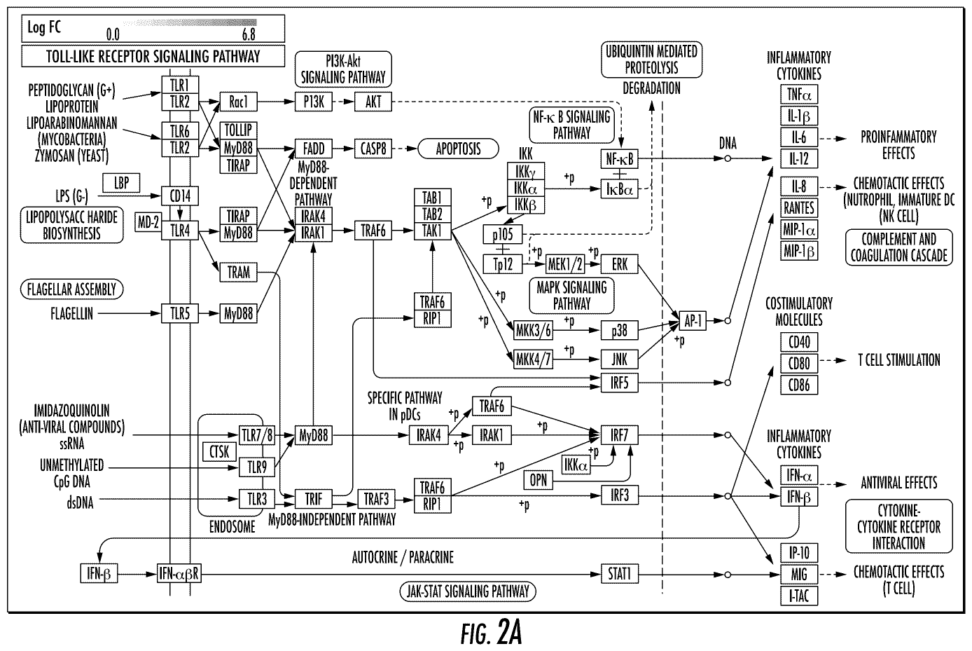

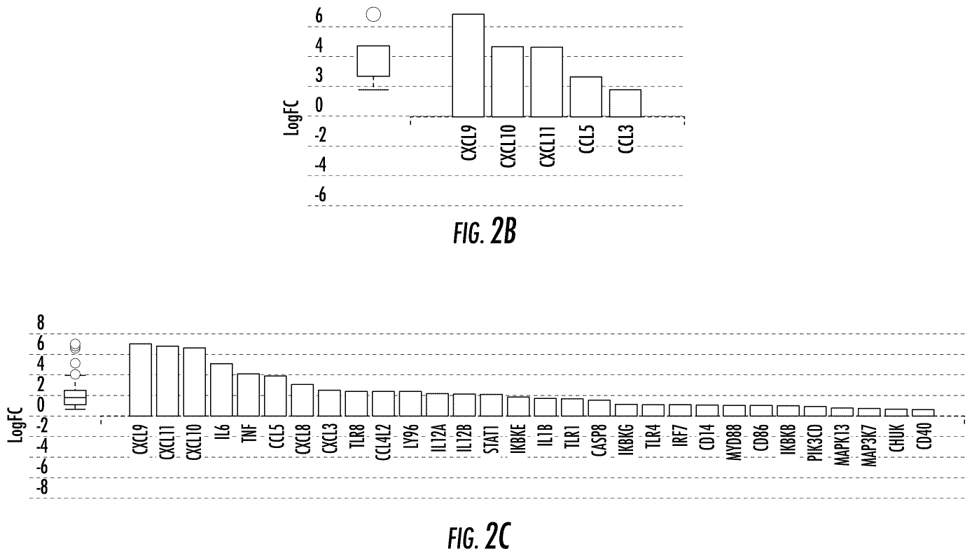

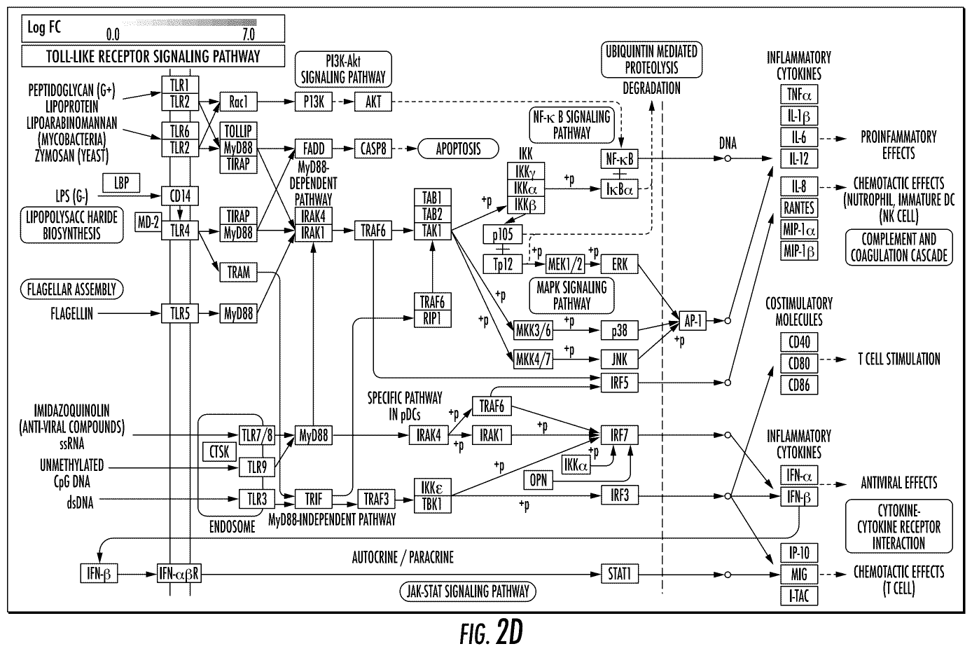

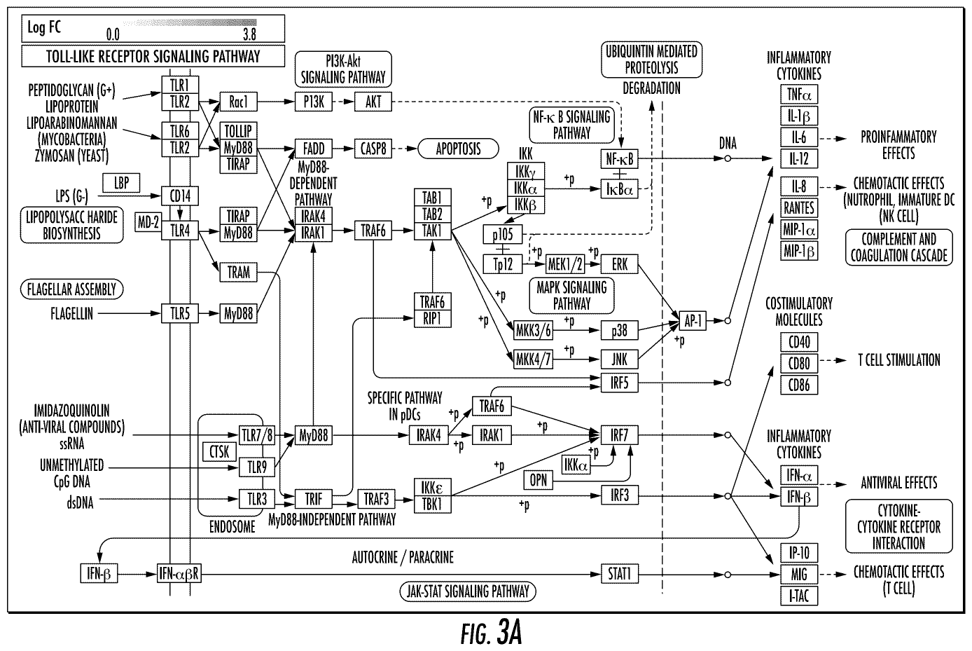

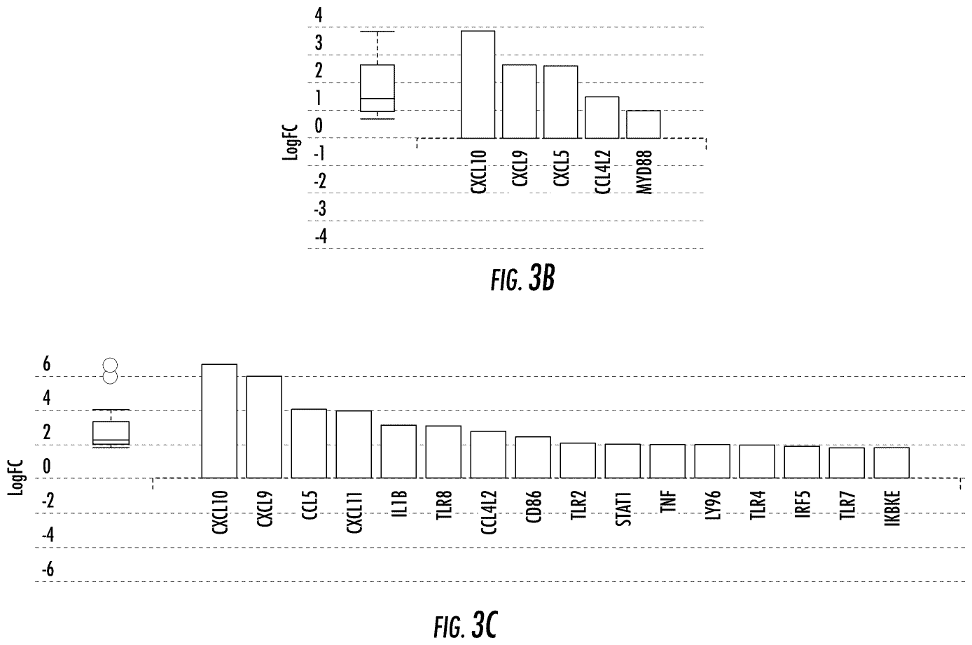

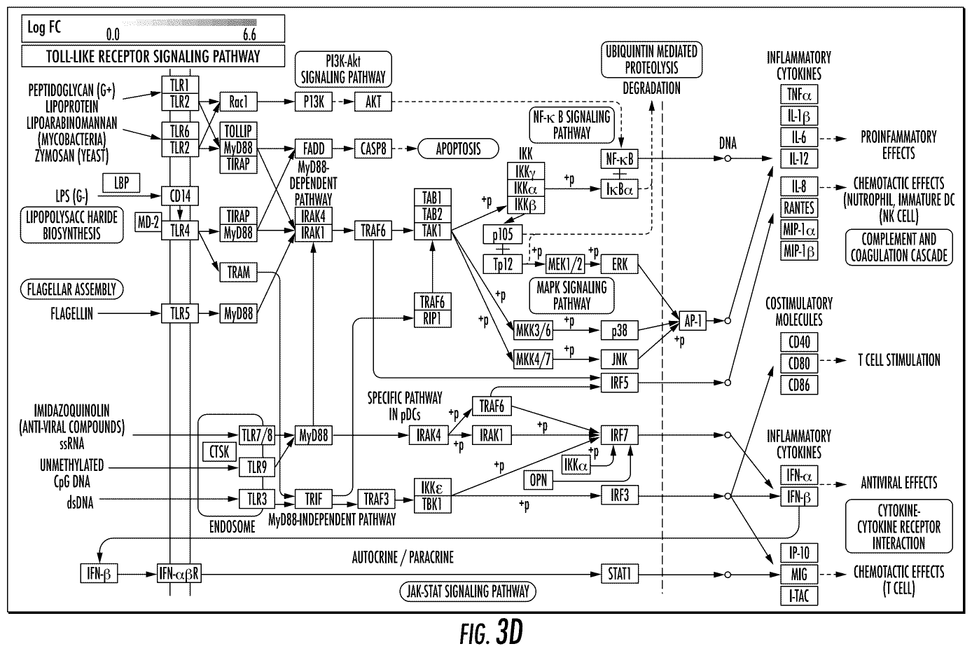

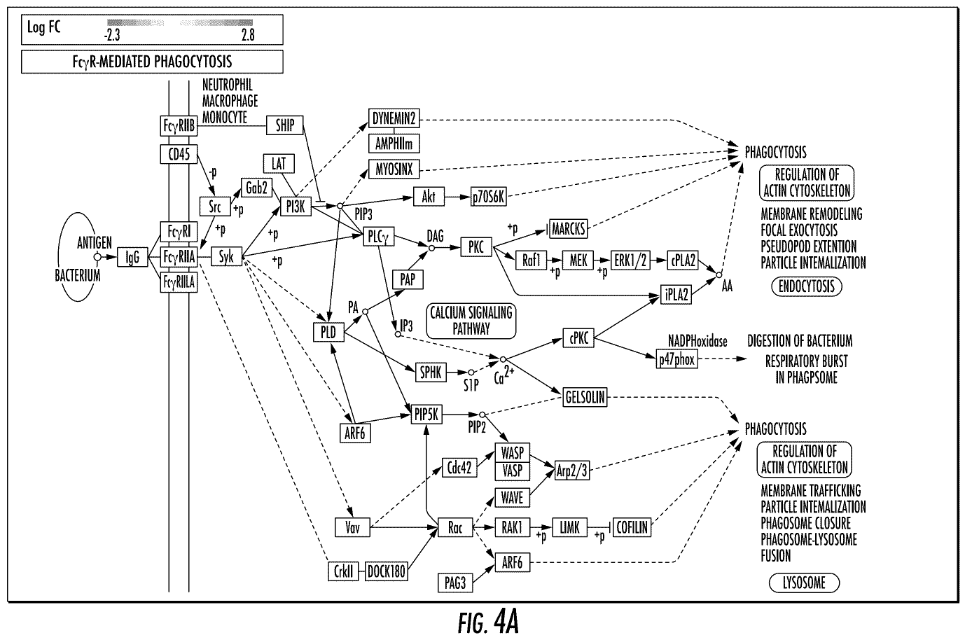

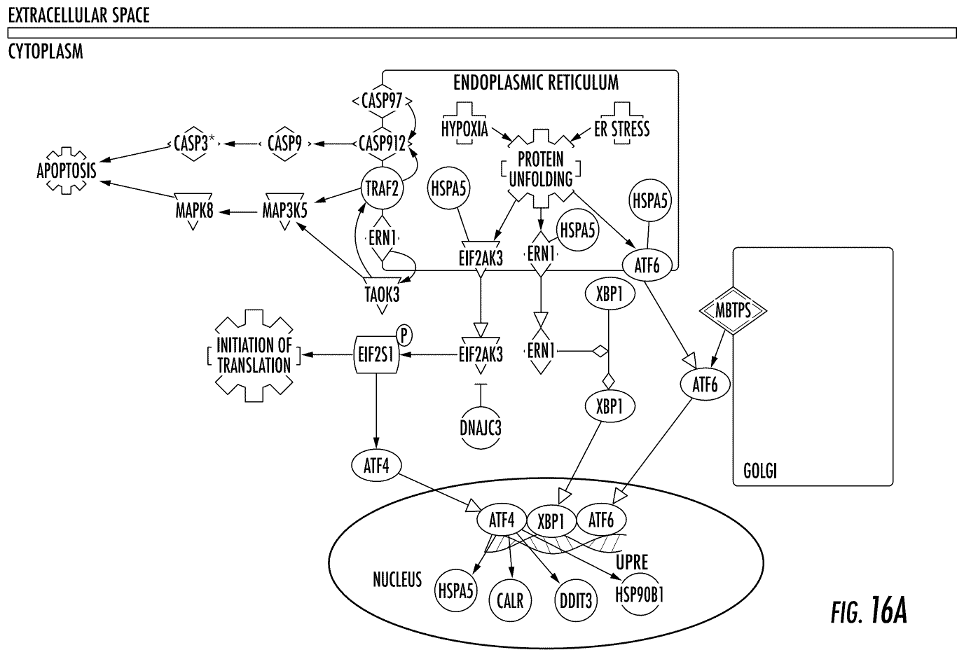

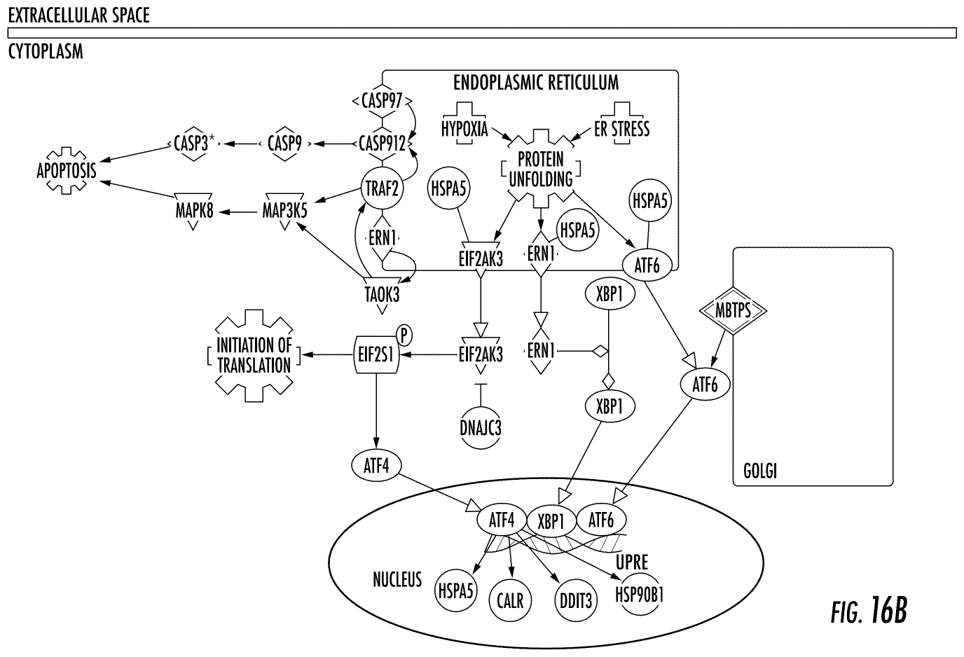

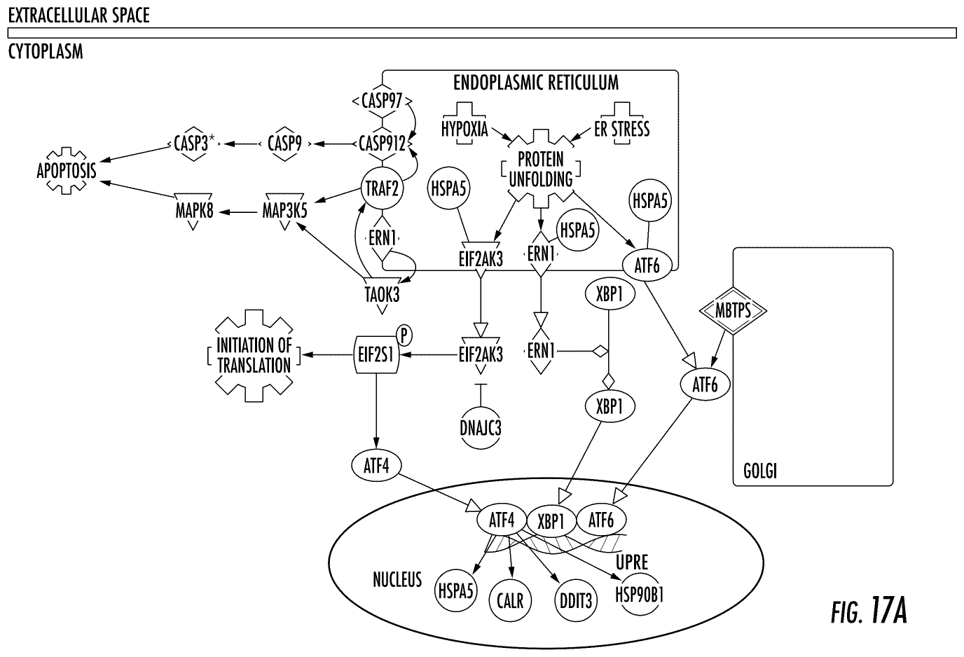

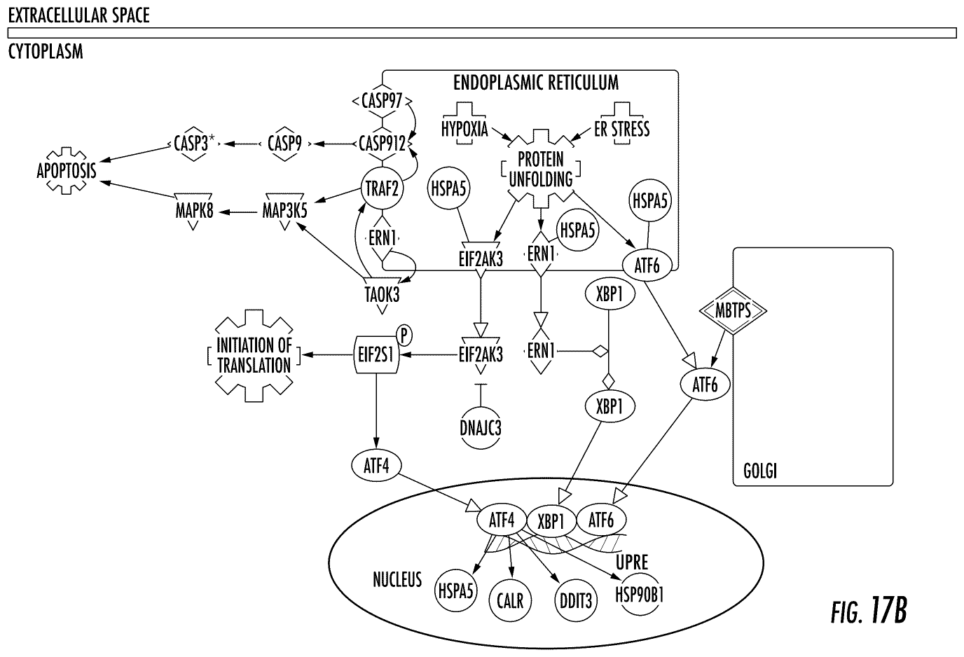

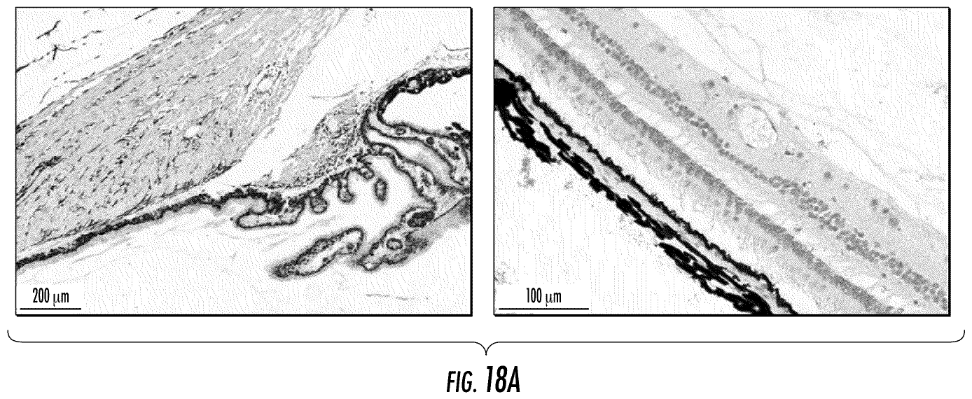

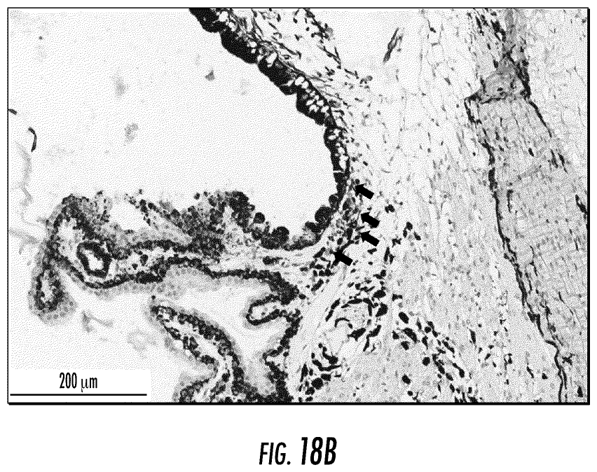

FIGS. 2A-17B, and 24A-25D illustrate the immune-related pathways and the corresponding gene signatures identified based on differential expression analysis of genes in those pathways induced in a dose dependent administration of Ixo-vec. FIGS. 2A-2C illustrate one exemplary analysis. FIG. 2A schematically illustrates the genes of the Toll-like Receptor Signaling Pathway and FIGS. 2B and 2C show plots of those genes from this pathway exhibiting differential expression above a threshold of log 2 greater than 0.6 in ciliary body-iris ocular tissue in response to either a low-dose (4E11 vg/eye) or high-dose (2E13 vg/eye) of Ixo-vec. Specific genes identified in the low-dose analysis are: CXCL9, CXCL10, CXCL11, CCL5 and CCL3. Specific genes identified in the high-dose analysis are: CXCL9, CXCL11, CXCL10, IL6, TNF, CCL5, CXCL8, CCL3, TLR8, CCL4L2, IY96, IL12A, IL12B, STAT1, IKBKE, IL1B, TLR1, CASP8, IKBKG, TLR4, IRF7, CD14, MYD88, CD86, IKBKB, KPK3CD, MP3K7, CHUK, and CD40. Similar differential expression analyses of data from dose-dependent rAAV studies in NHP are illustrated in FIGS. 3A-17B, and 24A-25D and described further in the Examples below. The resulting gene signatures for increased likelihood of rAAV-induced inflammatory response are summarized in Table 1 (below).

TABLE 1

| | | rAAV | |
|---|---|---|---|
| Pathway | Tissue | Dosage | Differentially Expressed Genes |
| Toll-like | ciliary | low | CXCL9, CXCL10, CXCL11, |
| Receptor | body-iris | | CCL5, CCL3 |
| Signaling | | high | CXCL9, CXCL11, CXCL10, |
| | | | IL6, TNF, CCL5, CXCL8, CCL3, |
| | | | TLR8, CCLAL2, IY96, IL12A, |
| | | | IL12B, STAT1, IKBKE, |
| | | | IL1B, TLR1, CASP8, |
| | | | IKBKG, TLR4, IRF7, |
| | | | CD14, MYD88, CD86, |
| | | | IKBKB, KPK3CD, MP3K7, |
| | | | CHUK, CD40 |
| | retina | low | CXCL10, CXCL9, CCL5, |
| | | | CCL4L2, MYD88 |
| | | high | CXCL10, CXCL9, CCL5, |
| | | | CXCL11, IL1B, TLR8, |
| | | | CCL4L2, CD86, TLR2, |
| | | | STAT1, TNF, LY97, |
| | | | TLR4, IRF7, TLR7, IKBKE |
| FcγR Mediated | ciliary | low | PLA2G4C, PRKCG, RAC2, |
| Phagocytosis | body-iris | | PTPRC, FCGR3A, |
| | | | FCGR2A, SYK, FCGR1A |
| | | high | PLA2G4C, PTPRC, RAC2, |
| | | | LAT, NCF1, FCGR3A, WAV1, |
| | | | SYK, PLA2G4B, WAS, |
| | | | PLCG2, HCK, FCGR2A, |
| | | | INPP5D, FCGR2A, |
| | | | INPP5D, FCGR1A, PIK3CD, |
| | | | PRKCD, VASP |
| | retina | low | RAC2, SYK, VAV2, FCGR3A, |
| | | | PTPRC |
| | | high | RAC2, FCGR2A, PTPRC, |
| | | | FCGR3A, SYK, |
| | | | VAV1, NCF1, PLA2G4C, |
| | | | WAS, SCIN, FCGR1A, |
| | | | INPP5D, HCK, PLD1 |
| Complement | ciliary | low | CIR, ITGAM, C4B, F13A1, |
| and | body-iris | | ITGB2, C1QC |
| Coagulation | | high | SERPINA5, CR1, CR2, |
| Cascades | | | C4B, ITGAX, PROC, |
| | | | ITGAM, C3, F12, F13A1, |
| | | | ITGB2, SERPINE1, |
| | | | PLAU, C1S, C1QC, CFHR4, |
| | | | C1QA, C1R |
| | retina | low | ITGAX, FGG, CPB2 |
| | | high | F13A1, ITGAX, C1QB, |
| | | | ITGAM, C1QC, C1QA, |
| | | | C2, ITGB2, C3, C4B, |
| | | | C1S, SERPINA1, VSIG4, |
| | | | SERPINA5, CR2, A2M, F2RL3 |
| Antigen | ciliary | low | HLA-DOB, KLRC2, CD8B, |
| Processing | body-iris | | TAP1, HLA-DRA, |
| and | | | CTSS, HLA-DMB, IFNG, |
| Presentation | | | CD8A, LGMN; |
| | | high | HLA-DOB, KLRD1, TNF, |
| | | | IFNG, TAP1, HLA-G, |
| | | | CIITA, KLRC2, CD8B, |
| | | | TAP2, HLA-E, CD8A, |
| | | | CTSS, HLA-DPB1, |
| | | | HLA-DPA1, HLA-DRB1, |
| | | | HLA-DMB, HLA-DRA, |
| | | | HLA-DOA, HLA-F, |
| | | | CD74, HLA-DMA, IFI30, |
| | | | PSME1, HSP90AA1, |
| | | | LGMN |
| | retina | low | HLA-DRB1, HLA-DQA1, |
| | | | HLA-DPA1, KLRD1 |
| | | high | HLA-DRA, HLA-G, B2M, |
| | | | HLA-DQA1, HLA- |
| | | | DRB1, HLA-DPB1, CIITA, |
| | | | CD74, HLA-DOA, |
| | | | HLA-DMB, HLA-DMA, |
| | | | HLA-E, CD8B, HLA- |
| | | | DPA1, CD4, TAP1, IFI30, |
| | | | KLRD1, HLA-DOB, |
| | | | TNF, CD8A, TAP2, PSME1 |
| B Cell Receptor | ciliary | low | CD79A, DAPP1, CD79B, |
| Signaling | body-iris | | CARD11, RAC2, |
| | | | BLNK, BTK, IFITIvII, SYK |
| | | high | CD79A, CD19, CARD11, |
| | | | CD79B, DAPP1, CR2, |
| | | | LILRA4, CD22, RAC2, |
| | | | BTK, LILRA1, BLNK, |
| | | | IFITM1, LILRB2, LILRB4, |
| | | | VAV1, SYK, PIK3AP1, |
| | | | PLCG2, INPP5D, CD72, IKBKG, |
| | | | IKBKB, PIK3CD, CHUK |
| | retina | low | DAPP1, RAC2, SYK, |
| | | | VAV2, NFATC2 |
| | | high | RAC2, CD79A, CD79B, |
| | | | DAPP1, LILRA4, SYK, |
| | | | VAV1, BTK, BLNK, |
| | | | LILRB2, INPP5D, |
| | | | NFATC2, LILRB4, |
| | | | CD22, CARD11, CR2, |
| | | | LILRA1, CD72, RASGRP3 |
| VEGF | ciliary | low | PLA2G4C, PRKCG, RAC2 |
| Signaling | body-iris | high | PLA2G4C, SH2D2A, RAC2, |
| | | | PLA2G4B, PLCG2, |
| | | | PIK3CD, MAPK13 |
| Aqueous Humor | ciliary | low | SLC12A5, SCN1A, |
| Production and | body-iris | | SCN9A, KCNA5 |
| Ciliary Body | | med | SLC4A9, NECTIN1, ATP1A3, |
| Fold | | | SLC4A10, SLC9A2, KCNA5, |
| Formation | | | SCN2A, SCN1A, KCNB2 |
| | | high | NECTIN1, KCNAB1, AQP9, |
| | | | ATP1A3, SCN9A, |
| | | | ATP1B3, KCNB2, SCN3B |
| T Cell Receptor | ciliary | low | LCK, CD40LG, CD3D, CD28, |
| Signaling | body-iris | | CD3E, CARD11, PAK5, CD247, |
| | | | CD8B, ITK, PTPRC, LCP2, |
| | | | IFNG, CD8A, FYN |
| | | high | LCK, CD3D, CARD11, |
| | | | CD28, TNF, ZAP70, |
| | | | CD40LG, CD3E, CD3G, |
| | | | PTPRC, ITK, IFNG, |
| | | | ICOS, PDCD1, LAT, LCP2, |
| | | | GRAP2, IL10, |
| | | | CD8B, CD8A, CD247, |
| | | | VAV1, PAK5, IKBKG, |
| | | | IKBKB, PIK3CD, MAPK13, |
| | | | MAP3K7, CHUK |
| | retina | low | VAV2, CD40LG, CD28, |
| | | | PTPRC, NFATC2 |
| | | high | CD3D, PTPRC, CD28, |
| | | | LCK, CD3G, VAV1, |
| | | | ICOS, CD8B, CD4, |
| | | | CD40LG, ITK, CD3E, TNF, |
| | | | CD8A, NFATC2, LCP2, |
| | | | CARD11, CTLA4 |

Based on their differential expression observed in tissue as a result of rAAV gene therapy treatments, the sets of differentially expressed genes from each of the immuno pathways in Table 1 can be used as a gene signature for the risk of rAAV-induced inflammation. Additionally, it is contemplated that sets of differentially expressed genes from combinations of the sets can be used as gene signatures for the risk of rAAV-induced inflammation. Thus, in at least one embodiment of the present disclosure, a set of genes comprising at least 2, at least 3, at least 4, at least 5, at least 6, at least 7, at least 8, at least 9, at least 10, or more genes selected from all of the differentially expressed genes disclosed in the sets of Table 1 can be used as a gene signature for the risk of rAAV-induced inflammation. Alternatively, in at least one embodiment of the present disclosure, a set of genes comprising 30 or fewer, 25 or fewer, 20 or fewer, 15 or fewer, 10 or fewer, or 5 or fewer genes selected from all of the differentially expressed genes disclosed in the sets of Table 1 can be used as a gene signature for the risk of rAAV-induced inflammation.

In another embodiment, it is contemplated that the gene signatures disclosed in the present disclosure can be used methods for screening, treating, and/or monitoring subjects for risk of an rAAV-induced inflammatory response using these gene signatures. For example, the gene signatures can be used in a method for screening subjects who are candidates for receiving an rAAV therapy and identify those subjects who are at risk of AAV-induced inflammation. Such a method would be carried out prior to receiving any treatment, such as administration of an initial dose of the rAAV composition. The method for screening the candidates for rAAV treatment would comprise: (a) obtaining a biological sample from the subject; (b) detecting expression levels of each of a set of genes in the biological sample (e.g., a set of differentially expressed genes of Table 1); (c) determining whether the detected expression levels of the set of genes indicate an increased likelihood of AAV-induced inflammation by comparing the measure expression levels to a predetermined threshold expression levels for the set of genes; and (d) identifying the subject as at risk of AAV-induced inflammation if the detected expression levels are above the threshold.

In at least one embodiment, the expression levels are determined using RNA sequencing and analysis methods, such as RNA-seq. One of ordinary skill can determine threshold expression levels using routine experimentation and analysis methods known in the art, such as those methods described in the Examples and elsewhere herein. For example, the threshold expression level can be log 2 fold-change in expression that is greater than a set level based on a control sample. In at least one embodiment, the threshold expression level can be log 2 fold-change in expression greater than |0.6|.

In addition to screening subjects prior to receiving an rAAV therapy, it is contemplated that the immune landscape gene signatures of the present disclosure can be used to treat and/or monitor patients who have received an rAAV-based gene therapy. For example, in at least one embodiment, the gene signatures of the present disclosure can be used in a method to identify a patient in need of an anti-inflammatory treatment from a population of patients who have received an rAAV therapy, such as intravitreal treatment with Ixo-vec. Such a method of would involve: (a) obtaining a biological sample from a patient who has received the rAAV treatment; (b) detecting expression levels of each of a set of genes in the biological sample (e.g., using RNA-seq analysis); (c) determining whether the detected expression levels indicate an increased likelihood of AAV-induced inflammation by comparison to predetermined threshold expression levels for the set of genes (e.g., a set of differentially expressed genes of Table 1); and (d) identifying the patient as in need of anti-inflammatory treatment if an increased likelihood of AAV-induced inflammation is determined based on the differential expression levels of the genes above the threshold. It is further contemplated that if an increased risk of AAV-induced inflammation is determined that the method can be further include a treatment step of administering an anti-inflammatory agent to the patient.

In at least one embodiment, the present disclosure provides a method of providing immunosuppressive treatment in a patient who has received intravitreal rAAV therapy, the method comprising; (a) obtaining a biological sample from the patient who has received an rAAV treatment; (b) detecting expression levels of each of a set of genes in the biological sample; (c) determining whether the detected expression levels indicate an increased likelihood of AAV-induced inflammation by comparison to predetermined threshold expression levels for the set of genes; and (d) administering an immunosuppressive treatment to the patient if an increased likelihood of AAV-induced inflammation is determined.

Treatments for various forms inflammation, including inflammation associated with gene therapies, such as rAAV-induced inflammation, are well-known in the art. In at least one embodiment, the anti-inflammatory agent is a steroid. Typical steroid for such treatments can include a corticosteroid, prednisone, and difluprednate. Depending on the particular rAAV-based treatment and how the inflammation is manifested, the treatment with a steroid can administered systemically, orally, topically, and/or intravitreally.

As noted above, the gene signatures and methods can be used to monitor patients who have received an rAAV-based gene therapy and are also receiving treatment for rAAV-induced inflammation. In such cases, the method can be used to monitor whether a patient's level of level of inflammation has changed significantly, ideally to a lower level such that the anti-inflammatory steroidal treatment administered can also be lowered. Generally, the gene signature expression level analysis used in the methods of the present disclosure allow for monitoring a subject/patient's rAAV-induced inflammation over time to see whether it has decreased (or increased) and use this information to adjust any treatment accordingly. Accordingly, in some embodiments, the biological sample obtained from the subject can include multiple samples obtained over time, for example days, weeks, months, or even years since initial treatment with the rAAV gene therapy, and/or any anti-inflammatory co-treatment. In at least one embodiment of the methods of the present disclosure, the biological sample can include a first biological sample obtained at a first time point and a second biological sample obtained at a second time point.

In addition to the use of the gene signatures for rAAV-induced inflammation in therapeutic methods, it is also contemplated that the gene signatures can be used in pre-clinical development methods for screening rAAV compositions for decreased rAAV-induced inflammation. Accordingly, in at least one embodiment the present disclosure provides a method for screening an rAAV composition for reduced AAV-induced inflammation comprising: (a) administering a dose of a test rAAV composition to a non-human subject; (b) obtaining a biological sample from the subject; (b) detecting expression levels of each of a set of genes in the biological sample; (d) comparing the detected expression levels for the test rAAV composition to expression levels detected for a dose of a control rAAV composition. It is contemplated that such pre-clinical rAAV screening could be used in animal models, cell-based assays, or in vitro models to determine whether a particular rAAV composition exhibited improved characteristics of reduced likelihood of inducing an inflammatory response in a human patient.

### EXAMPLES

Various features and embodiments of the disclosure are illustrated in the following representative examples, which are intended to be illustrative, and not limiting. Those skilled in the art will readily appreciate that the specific examples are only illustrative of the invention as described more fully in the claims which follow thereafter. Every embodiment and feature described in the application should be understood to be interchangeable and combinable with every embodiment contained within.

### Example 1: Construction of Gene Annotation and Differential Expression Analysis

This example illustrates studies using transcriptomic analysis of dose-response data obtained from NHP subjects to identify pathways, and gene signatures associated with increased risk of rAAV-induced inflammatory response in humans. Briefly, three months following IVT administration in a single eye of Ixo-vec to cynomolgus macaque NHPs in the choroid, retina, and ciliary body/iris tissues were isolated for bulk RNA sequence analysis. After determining log-fold changes in gene expression, pathway analysis was performed using three different software tools: (1) Immune Landscape Signature (ILS) analysis; (2) Ingenuity Pathway Analysis (IPA); and (3) iPathwayGuide (IPG) analysis. Further correlative histological analysis was performed to validate the findings of pathway analysis.

### Materials and Methods

### A. RNA Sequence Analysis

RNA was extracted from choroid, retina, and ciliary body-iris tissues (RNeasy kit; Qiagen) and used for bulk RNA-sequencing using TruSeq stranded mRNA kit (Illumina). After read preparation and quality control analysis using fastp (fastp v016.4; available at github.com/OpenGen/fastp), trimmed and filtered reads were aligned to the cynomolgus macaque (M. fascicularis) reference genome, macfas5, available from Ensembl using the STAR aligner (v2.6.1d).

### B. Analysis RNA Abundances

Transcript abundances were quantified using the RSEM software package (v1.3.1). For two group differential expression analysis, 5 different publicly available tools were run for each gene: DESeq2, edgeR, EBSeq, limma, and t-test.

### C. Identifying Differential Expressed Genes (DEGs)

Pairwise differential expression testing was performed using Q² Solutions' Ensemble two-group comparisons suite (Q² Solutions LLC; Durham, North Carolina, US; www.q2labsolutions.com/bioinformatics). Briefly, Q² Solutions' Ensemble method summarizes the differential expression p-values and classification probabilities from five popular tools (t-test, limma, DESeq2, edgeR and EBSeq) to produce a new p-value for differential expression which has demonstrated proper Type I error control and superior sensitivity. Aspects of each component test were utilized as input to a logistic regression model trained on data from The Cancer Genome Atlas (TCGA) which produces an estimate of the probability that a gene is differentially expressed between two conditions. This value is further transformed to a proper p-value by comparison against its empirical cumulative distribution under the null established via bootstrap resampling of TCGA data from various cancer types. From this p-value, a list of differentially expressed genes (DEGs) were identified for each two-group comparison.

### D. Identifying Human Homologs of DEGs

After first mapping, annotating the dataset to cynomolgus transcriptome, and determining DEGs as described above, the human gene homologues were identified using Ensembl (available at: www.ensembl.org). To improve gene annotation, biomaRt R package was utilized. BiomarRt is an expert curated database of gene annotations across multiple organisms with data compiled from various consortia (see e.g., Durinck et al., “BioMart and Bioconductor: a powerful link between biological databases and microarray data analysis,” Bioinformatics Applications Note, Vol. 21 no. 16 2005, pages 3439-3440, doi:10.1093/bioinformatics/bti525; and Durinck et al., “Mapping Identifiers for the Integration of Genomic Datasets with the R/Bioconductor package biomaRt,” Nat Protoc. 2009; 4(8): 1184-1191. doi:10.1038/nprot.2009.97). For this project, the M. fascicularis human homologs were identified using the Ensembl genomes.

### E. Pathway Analyses

Pathway analysis was performed to identify the pathways that are significantly impacted by a given treatment and to understand how these pathways produce a phenotypic effect. Three different software tools were run to evaluate rAAV vector dose-response across treatment groups: 1. Immune landscape signature (ILS) scores (Q2 Solutions), 2. Ingenuity Pathway Analysis (Qiagen), and 3. iPathwayGuide (Advaita Biosciences).

1. Immune Landscape Signature (ILS): ILS scores were calculated from normalized RNA-seq data and characterized immune cell subtypes, individual genes, and pathways. Gene sets that co-express together and are associated with specific immune cell subtypes were generated from a database of RNA expression tumor samples from multiple cancer indications (The Cancer Genome Atlas). All ILS show a correlation with event free survival, overall survival, or both in numerous cancer types independent of the treatment type (see e.g., Jones et al., “RNA Immune Signatures from Pan-Cancer Analysis Are Prognostic for High-Grade Serous Ovarian Cancer and Other Female Cancers,” Cancers 2020, 12, 620; doi:10.3390/cancers12030620).

2. Ingenuity Pathway Analysis (IPA): IPA analyses were performed using the human homolog dataset. IPA is a non-topology-based, or gene set analysis, method, which uses two enrichment analysis scores to determine the significance of upstream regulators and networks associated with DEGs. One score is determined by over-presentation analysis (ORA) which compares the number genes associated with a given pathway to the list of DEGs. Fisher's exact test is the type of ORA used to calculate the probability of the number of observed DEGs in a pathway being observed by chance. If the pathway contains significantly more than expected DEGs, it is likely to be a truly related to a condition. The second enrichment score is determined by a Z-score which evaluates extent to which predicted up/down regulation patterns match those observed (Kramer 2013). Thresholds for log 2 fold-change in expression was set to be >10.61. Unadjusted p-value threshold was <0.05. Only mammalian species and only tissues (not cells from in vitro studies) were selected as references for core analyses. Default setting was used for all other parameters.

3. iPathwayGuide (IPG): IPG analyses were performed using the human homolog dataset. IPG is a topology-based, or impact analysis, method that uses two sources of information to measure the impact on a given pathway. One method is to calculate the over-representation of DEGs within the pathway. The second method incorporates biological factors pertaining to every single interaction within the pathway. Specific biological factors considered are the position of each DEG in the pathway, the amount of expression change for all DEGs, and the known roles of each gene on the pathway. Roles are detailed by the interactions between genes on the pathway and the type of interactions (Nguyen 2019). Thresholds for log 2 fold-change in expression was set to be >10.61. Unadjusted p-value threshold was <0.05.

### 4. Correlative Histological Analysis:

Anterior tissues were examined by immunohistochemistry (IHC) for the presence of CD20+ B cells and CD138+ plasma cells. The presence of CD4+ and CD8+ T cells were also examined in anterior tissues by IHC. The following primary antibodies were used: anti-CD20 (Thermo Fisher; cat. #PA5-16701) at 0.26 g/mL, anti-CD138 (Agilent; cat. #GA64261-2) at 0.2 μg/mL, anti-CD4 (abcam; cat. #AB133616) at 0.29 μg/mL, and anti-CD8 (LS Bio; cat. #LS-B3914) at 200 μg/mL.

5. Whole blood analysis: Peripheral whole blood was collected at multiple time points after dosages of IVT administered Ixo-vec and stored in cryotubes maintained at −80° C. until further use. RNA was extracted from whole blood using NucleoSpin RNA Blood kit (Takara Bio; cat. #740200.50). Frozen samples were thawed at room temperature with an equal volume of lysis buffer DL. Once the blood was thawed, the blood-DL mixture was aliquoted into new, sterile microcentrifuge tubes at volumes of 800 μL. The aliquots were then processed using the protocol for an input of 400 μL of blood, per the manufacturer's instructions, with a deviation made by adding a 4th wash of the silica membrane using 250 μL of wash buffer RB3.

### Results

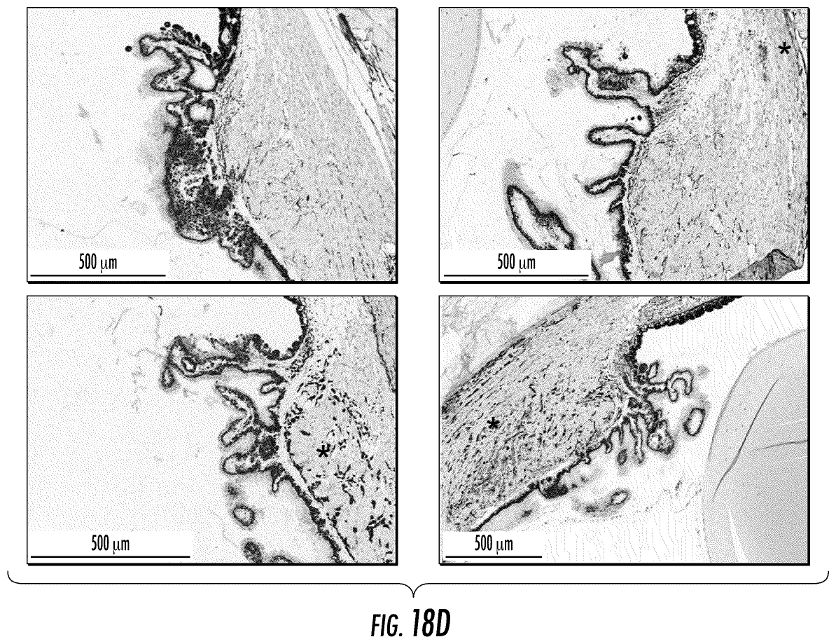

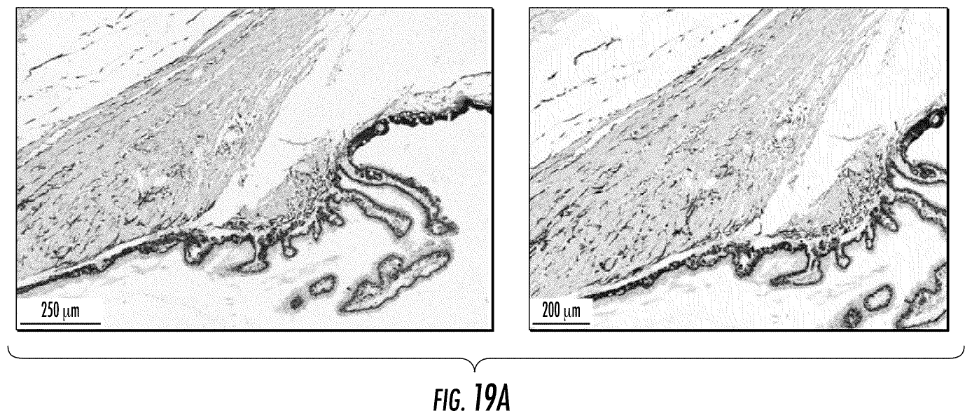

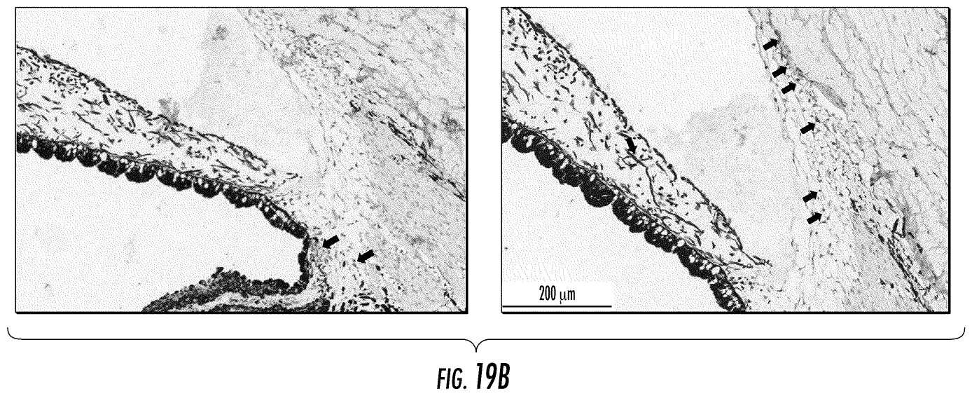

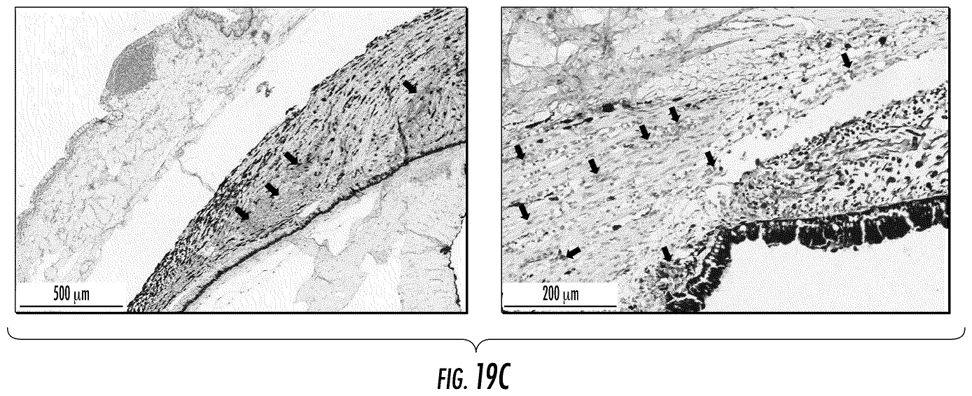

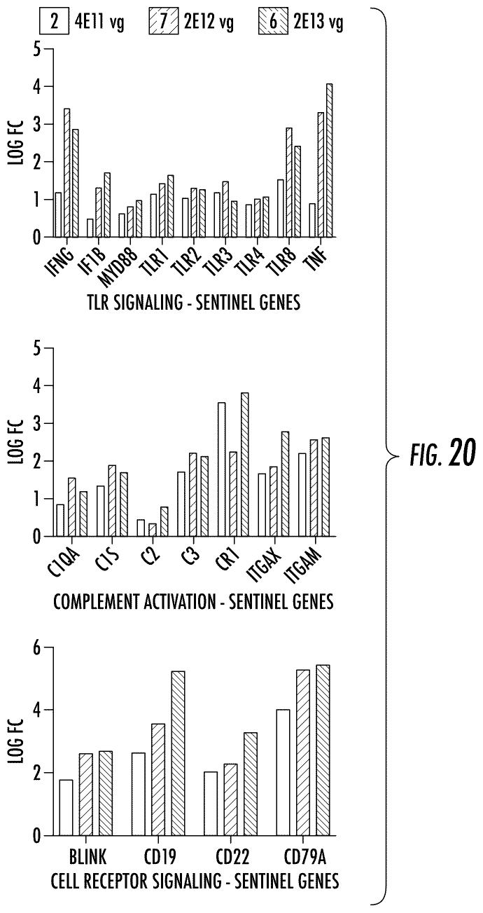

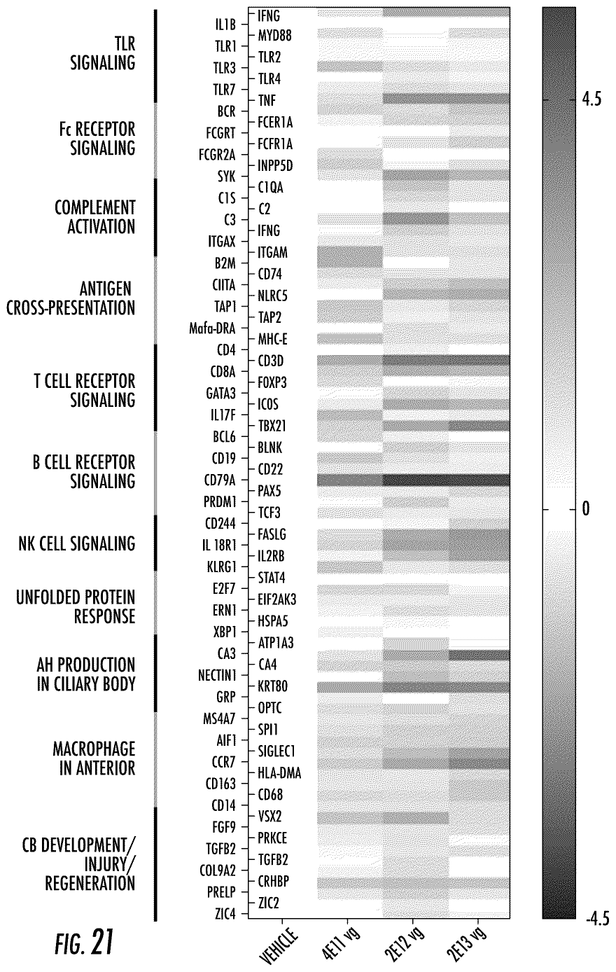

Transcriptomic analysis did not support UPR, oxidative or ER stress, neovascularization, nor ciliary body dysfunction. Unfolded protein and endoplasmic reticulum stress responses (UPR) were not upregulated, indicating that toxicity due to overproduction of exogenous aflibercept did not cause inflammation at higher doses. Furthermore, there was no evidence of disrupted ciliary body architecture or VEGF axis dysregulation. RNA-seq and histopathology indicated activation of innate and adaptive immune systems consistent with dose-dependent rAAV-associated inflammation. Histological analysis demonstrated dose-dependent increase in mononuclear infiltrates, including CD4+ T-cells and CD20+ B-cells (see e.g., FIGS. 18A-18D and FIGS. 19A-19C).

Ixo-vec expression was detected only in the dosed eye. No alternative splicing was observed. Robust and dose-dependent activation of immune responses were observed, consistent in nature across ocular tissues. Pathways showed activation at 4E11 vg/eye but were activated to a greater extent in higher dosed groups. Despite adaptive immune response, Ixo-vec expression levels plateaued across doses. A dose-response was observed predominately in anterior tissues of the eye and in the retina, and most pathways were found unperturbed in choroid. Based on the results described herein, use of lower doses and transient immunosuppression regimen will likely prevent ocular inflammation in response to Ixo-vec.

Pathway analysis and IHC converged on immune response pathways activated in response to rAAV. Upregulation of hallmark genes in the inflammatory signatures were confirmed by RT-qPCR. Further detailed results of the different specific analyses in identifying the same immune-related pathways of interest and DEGs as dysregulated in a dose-dependent manner by Ixo-vec in various ocular tissues are provided below and in the accompanying figures.

FIGS. 2A, 2B, 2C, 2D, 3A, 3B, 3C, and 3D show results identifying signatures that have overlapping genes expressed in the toll-like receptor signaling pathway, including NK cell signature (NK), IL pro-inflammatory signaling, IL6, and T cell trafficking signature.

As shown by the pathway diagram of FIG. 2A and the plot of FIG. 2B, the following toll-like receptor pathway genes were identified as DEGs in ciliary body-iris tissue in response to the 4E11 vg/eye dose of Ixo-vec: CXCL9, CXCL10, CXCL11, CCL5 and CCL3. As shown by the plot of FIG. 2C and the pathway diagram of FIG. 2D, the following toll-like receptor pathway genes were identified as DEGs in ciliary body-iris tissue in response to the 2E13 vg/eye dose of Ixo-vec: CXCL9, CXCL11, CXCL10, IL6, TNF, CCL5, CXCL8, CCL3, TLR8, CCL4L2, IY96, IL12A, IL12B, STAT1, IKBKE, IL1B, TLR1, CASP8, IKBKG, TLR4, IRF7, CD14, MYD88, CD86, IKBKB, KPK3CD, MP3K7, CHUK, and CD40.

As shown by the pathway diagram of FIG. 3A and the plot of FIG. 3B, the following toll-like receptor pathway genes were identified as DEGs in retina tissue in response to the 4E11 vg/eye dose of Ixo-vec: CXCL9, CXCL10, CXCL9, CCL5, CCL4L2, and MYD88. As shown by the plot of FIG. 3C and the pathway diagram of FIG. 3D, the following toll-like receptor pathway genes were identified as DEGs in retina tissue in response to the 2E13 vg/eye dose of Ixo-vec: CXCL10, CXCL9, CCL5, CXCL11, IL1B, TLR8, CCL4L2, CD86, TLR2, STAT1, TNF, LY97, TLR4, IRF7, TLR7 and IKBKE.

TLR1, TLR2, and TLR4 have been reported to be activated by viral proteins. The results here suggest that TLR8 also is activated by viral transcripts. The results also suggest that unmethylated CpG DNA-TLR9-MYD88 axis is not activated.

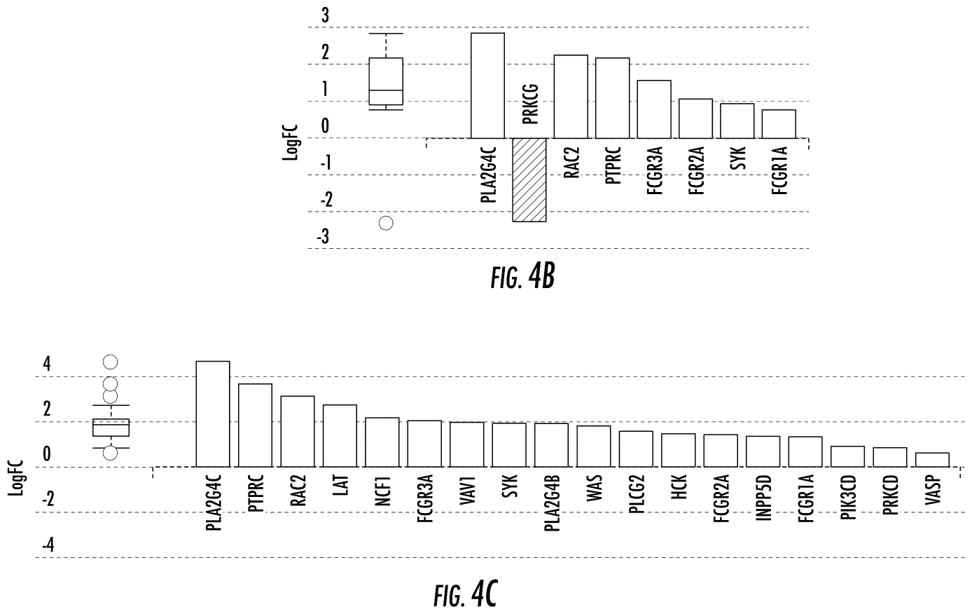

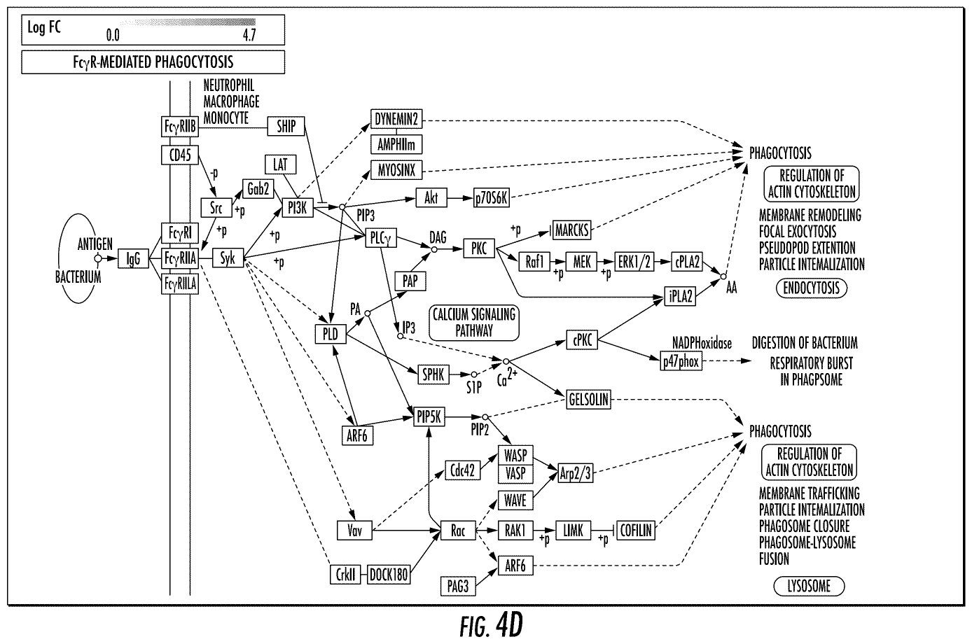

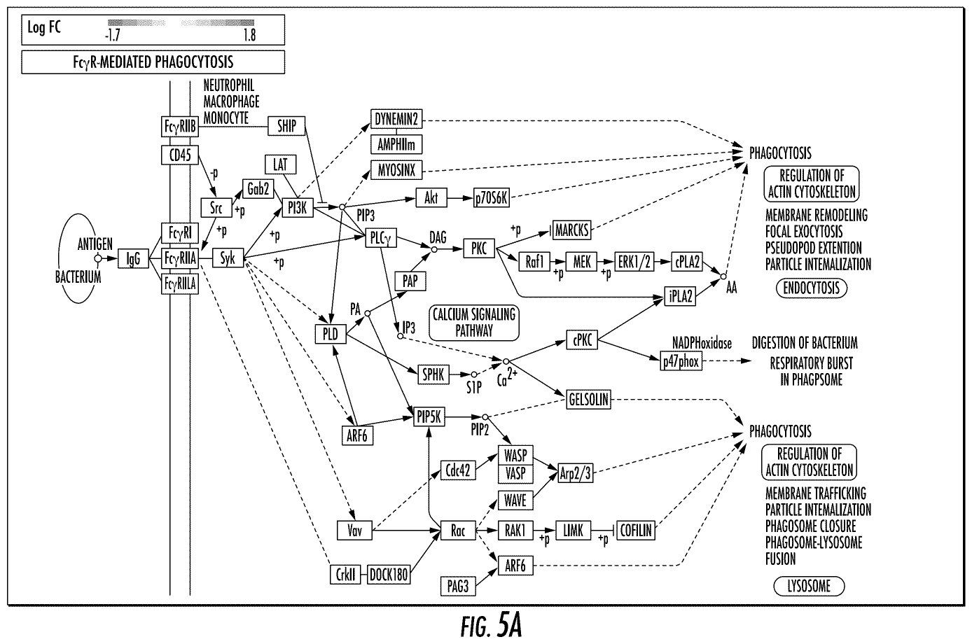

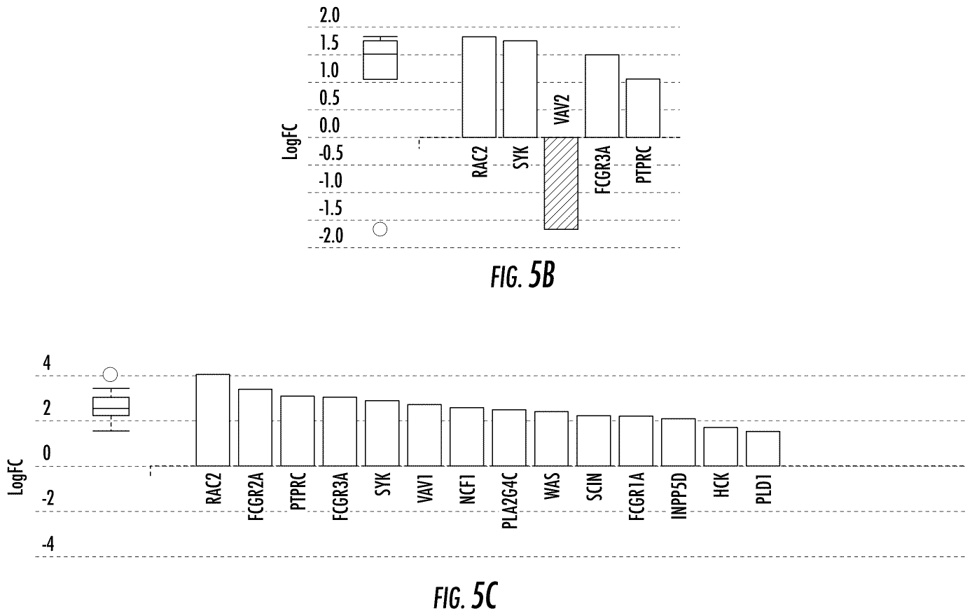

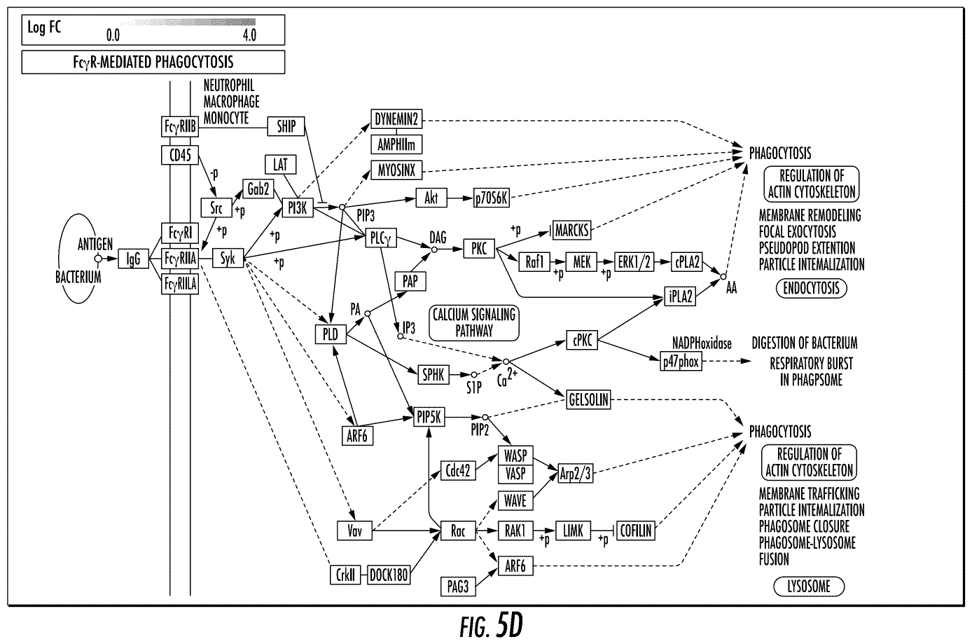

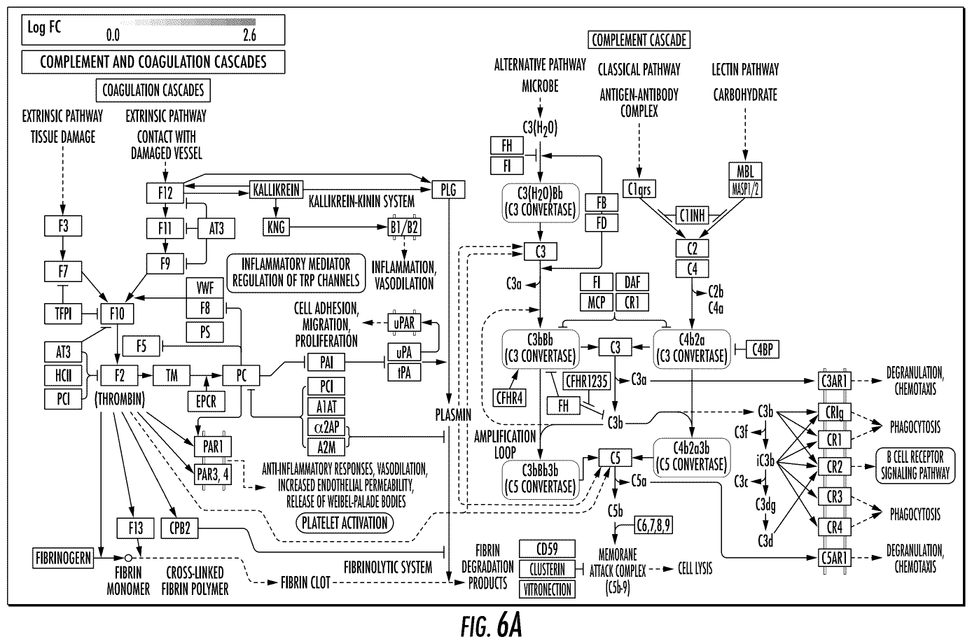

FIGS. 4A, 4B, 4C, 4D, 5A, 5B, 5C, and 5D show results identifying signatures that have overlapping genes expressed in Fc-gamma receptor-mediated phagocytosis pathway, including NK cell signature (NK), M1 macrophage signature, IFNG, M2 TAM macrophage signature, B cell signature, CD8+ T cell signature (CD8), and cytotoxic lymphocyte immune signature (CLIS).

As shown by the pathway diagram of FIG. 4A and the plot of FIG. 4B, the following Fc-gamma receptor-mediated phagocytosis pathway genes were identified as DEGs in ciliary body-iris tissue in response to the 4E11 vg/eye dose of Ixo-vec: PLA2G4C, PRKCG, RAC2, PTPRC, FCGR3A, FCGR2A, SYK, and FCGR1A. As shown by the plot of FIG. 4C and the pathway diagram of FIG. 4D, the following Fc-gamma receptor-mediated phagocytosis pathway genes were identified as DEGs in ciliary body-iris tissue in response to the 2E13 vg/eye dose of Ixo-vec: PLA2G4C, PTPRC, RAC2, LAT, NCF1, FCGR3A, WAV1, SYK, PLA2G4B, WAS, PLCG2, HCK, FCGR2A, INPP5D, FCGR2A, INPP5D, FCGR1A, PIK3CD, PRKCD, and VASP.

As shown by the pathway diagram of FIG. 5A and the plot of FIG. 5B, the following Fc-gamma receptor-mediated phagocytosis pathway genes were identified as DEGs in retina tissue in response to the 4E11 vg/eye dose of Ixo-vec: RAC2, SYK, VAV2, FCGR3A and PTPRC. As shown by the plot of FIG. 5C and the pathway diagram of FIG. 5D, the following Fc-gamma receptor-mediated phagocytosis pathway genes were identified as DEGs in retina tissue in response to the 2E13 vg/eye dose of Ixo-vec: RAC2, FCGR2A, PTPRC, FCGR3A, SYK, VAV1, NCF1, PLA2G4C, WAS, SCIN, FCGR1A, INPP5D, HCK and PLD1.

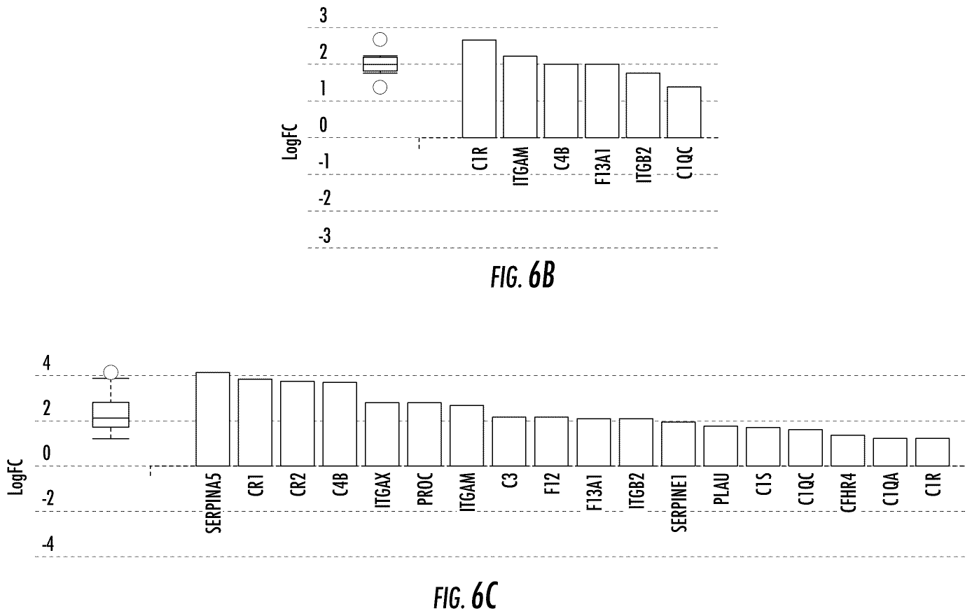

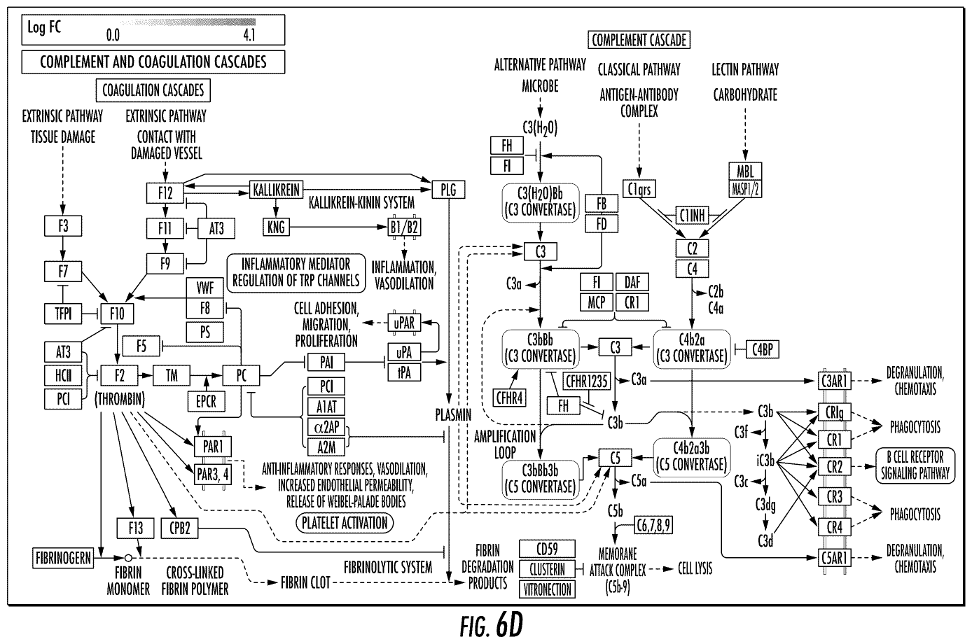

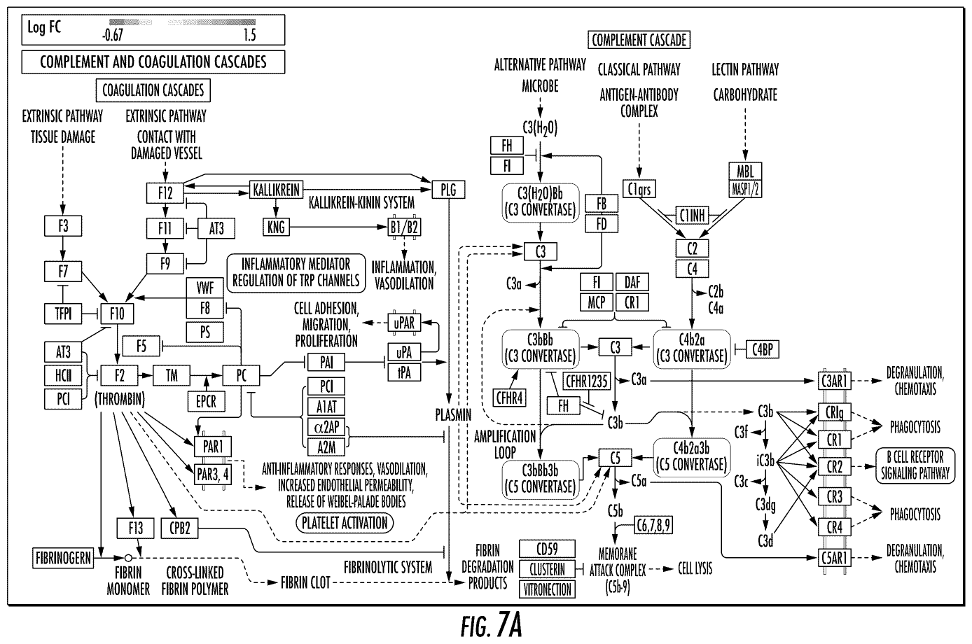

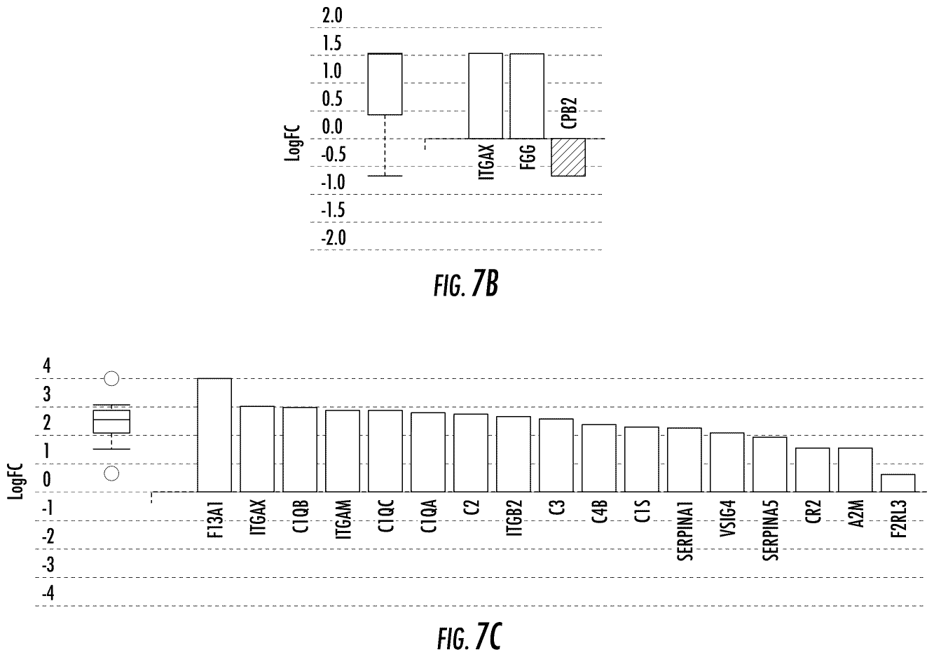

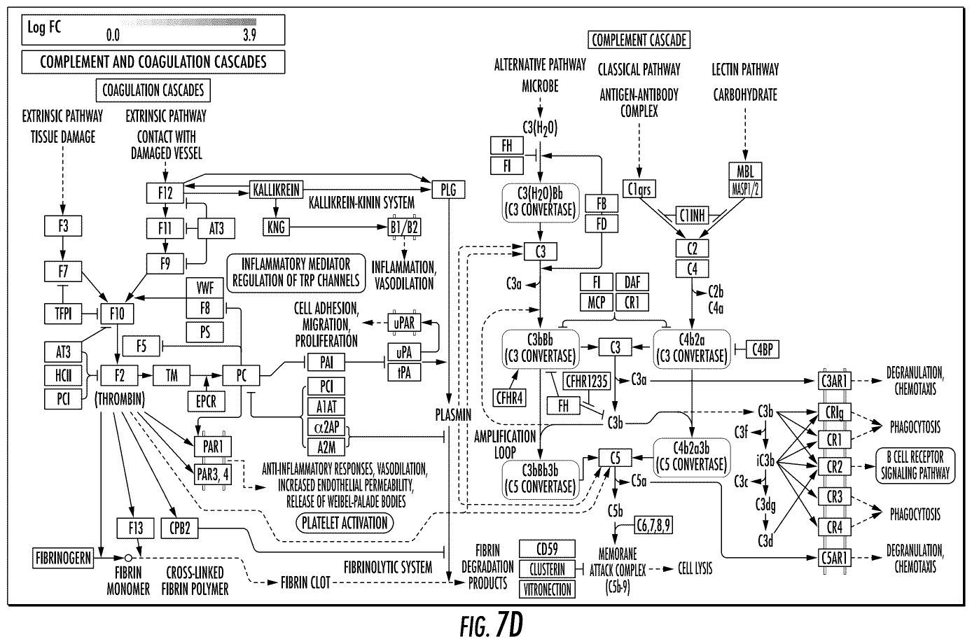

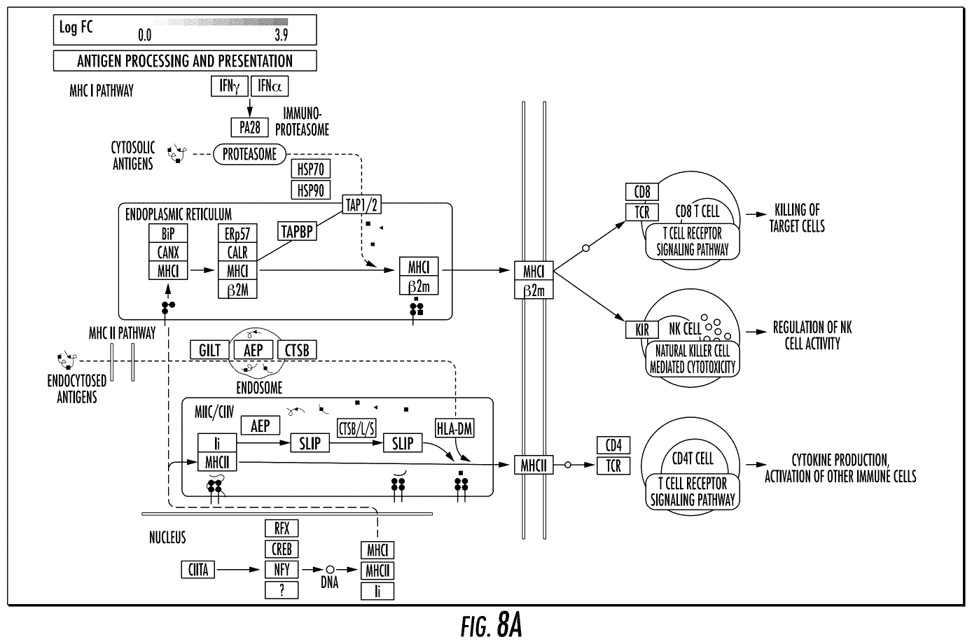

C1 complex-mediated response was observed in a dose-responsive manner. FIGS. 6A, 6B, 6C, 6D, 7A, 7B, 7C, and 7D show results identifying signatures that have overlapping genes expressed in complement and coagulation cascade pathway including NK cell signature (NK), M1 macrophage signature, M2 TAM macrophage signature, and B cell signature.

As shown by the pathway diagram of FIG. 6A and the plot of FIG. 6B, the following complement and coagulation cascade pathway genes were identified as DEGs in ciliary body-iris tissue in response to the 4E11 vg/eye dose of Ixo-vec: C1R, ITGAM, C4B, F13A1, ITGB2 and C1QC. As shown by the plot of FIG. 6C and the pathway diagram of FIG. 6D, the following complement and coagulation cascade pathway genes were identified as DEGs in ciliary body-iris tissue in response to the 2E13 vg/eye dose of Ixo-vec: SERPINA5, CR1, CR2, C4B, ITGAX, PROC, ITGAM, C3, F12, F13A1, ITGB2, SERPINE1, PLAU, C1S, C1QC, CFHR4, C1QA and C1R.

As shown by the pathway diagram of FIG. 7A and the plot of FIG. 7B, the following complement and coagulation cascade pathway genes were identified as DEGs in retina tissue in response to the 4E11 vg/eye dose of Ixo-vec: ITGAX, FGG and CPB2. As shown by the plot of FIG. 7C and the pathway diagram of FIG. 7D, the following complement and coagulation cascade pathway genes were identified as DEGs in retina tissue in response to the 2E13 vg/eye dose of Ixo-vec: F13A1, ITGAX, C1QB, ITGAM, C1QC, C1QA, C2, ITGB2, C3, C4B, C1S, SERPINA1, VSIG4, SERPINA5, CR2, A2M, and F2RL3.

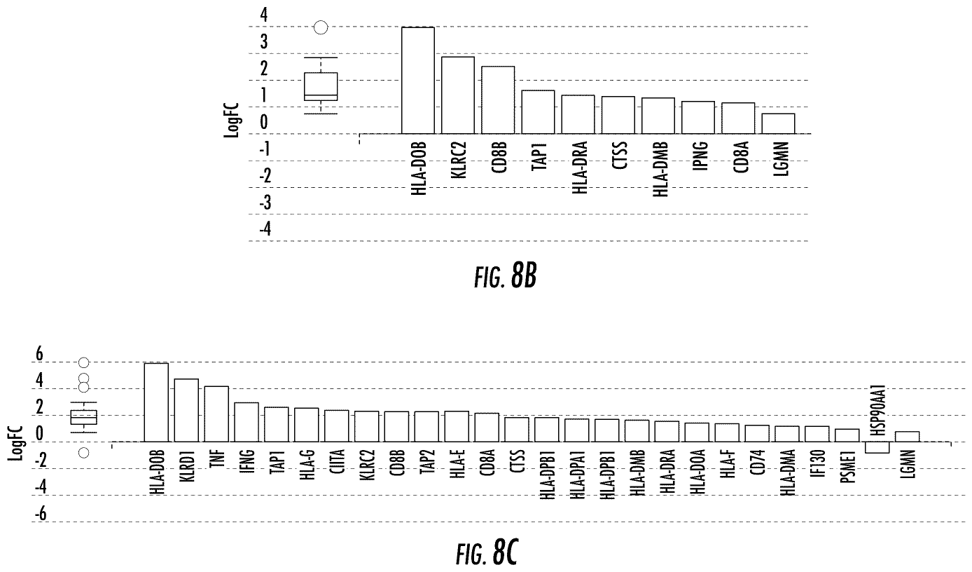

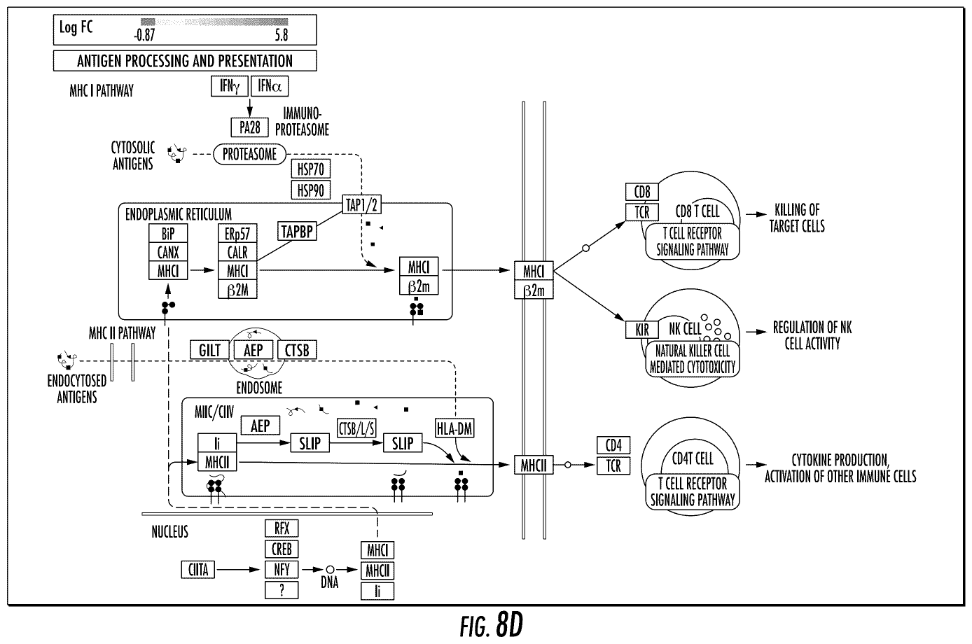

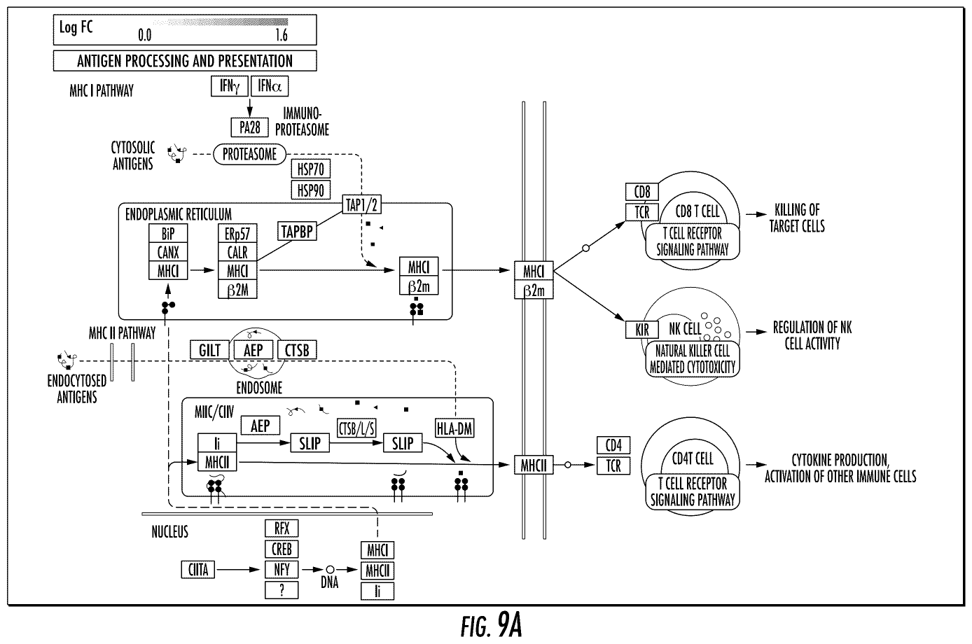

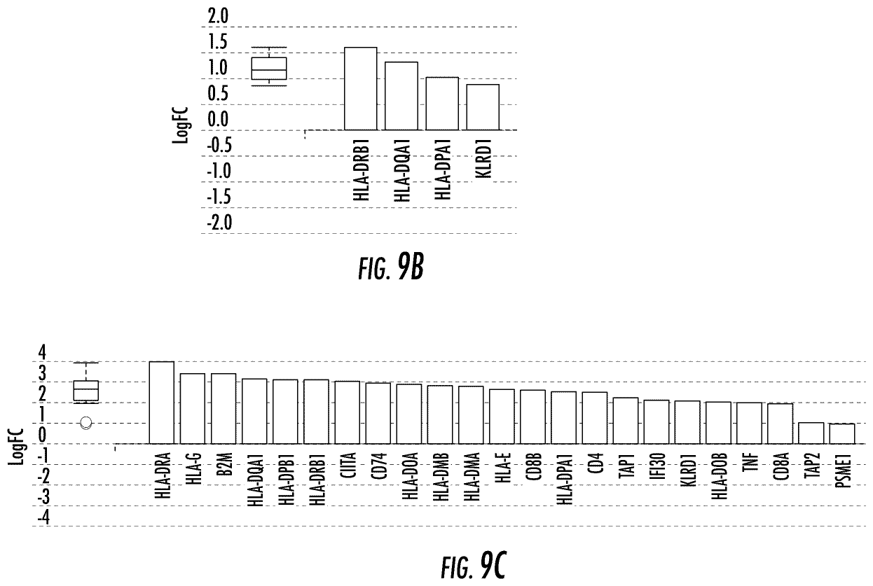

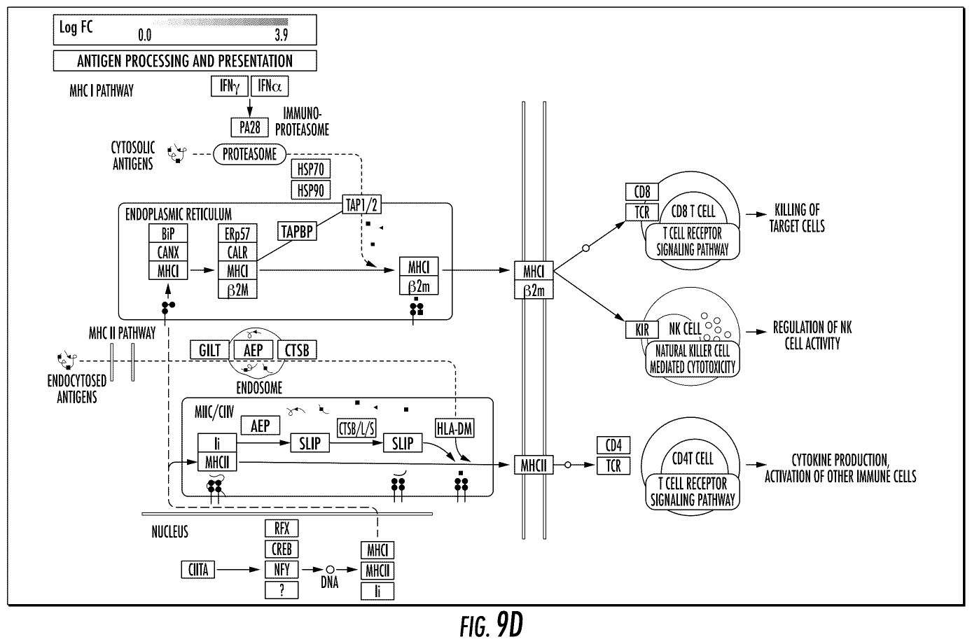

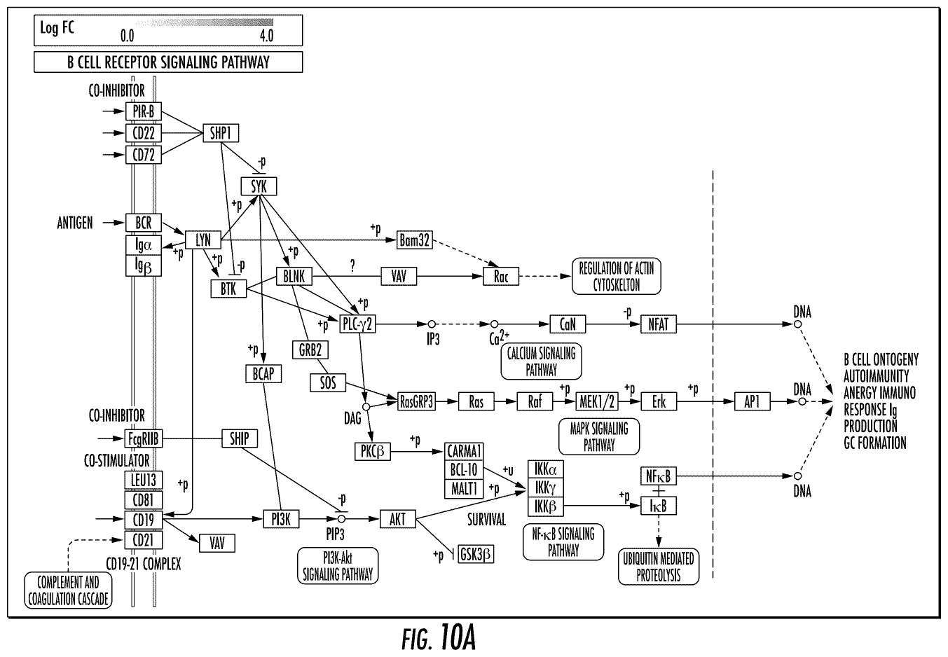

FIGS. 8A, 8B, 8C, 8D, 9A, 9B, 9C, and 9D show results identifying signatures that have overlapping genes expressed in antigen processing and presentation pathway including NK cell signature (NK), IL pro-inflammatory signature, IFNG, B cell signature, CD8+ T cell signature (CD8), and cytotoxic lymphocyte immune signature (CLIS).

As shown by the pathway diagram of FIG. 8A and the plot of FIG. 8B, the following antigen processing and presentation pathway genes were identified as DEGs in ciliary body-iris tissue in response to the 4E11 vg/eye dose of Ixo-vec: HLA-DOB, KLRC2, CD8B, TAP1, HLA-DRA, CTSS, HLA-DMB, IFNG, CD8A and LGMN. As shown by the plot of FIG. 8C and the pathway diagram of FIG. 8D, the following antigen processing and presentation pathway genes were identified as DEGs in ciliary body-iris tissue in response to the 2E13 vg/eye dose of Ixo-vec: HLA-DOB, KLRD1, TNF, IFNG, TAP1, HLA-G, CIITA, KLRC2, CD8B, TAP2, HLA-E, CD8A, CTSS, HLA-DPB1, HLA-DPA1, HLA-DRB1, HLA-DMB, HLA-DRA, HLA-DOA, HLA-F, CD74, HLA-DMA, IFI30, PSME1, HSP90AA1, and LGMN.

As shown by the pathway diagram of FIG. 9A and the plot of FIG. 9B, the following antigen processing and presentation pathway genes were identified as DEGs in retina tissue in response to the 4E11 vg/eye dose of Ixo-vec: HLA-DRB1, HLA-DQA1, HLA-DPA1, and KLRD1. As shown by the plot of FIG. 9C and the pathway diagram of FIG. 9D, the following antigen processing and presentation pathway genes were identified as DEGs in retina tissue in response to the 2E13 vg/eye dose of Ixo-vec: HLA-DRA, HLA-G, B2M, HLA-DQA1, HLA-DRB1, HLA-DPB1, CIITA, CD74, HLA-DOA, HLA-DMB, HLA-DMA, HLA-E, CD8B, HLA-DPA1, CD4, TAP1, IFI30, KLRD1, HLA-DOB, TNF, CD8A, TAP2, and PSME1.

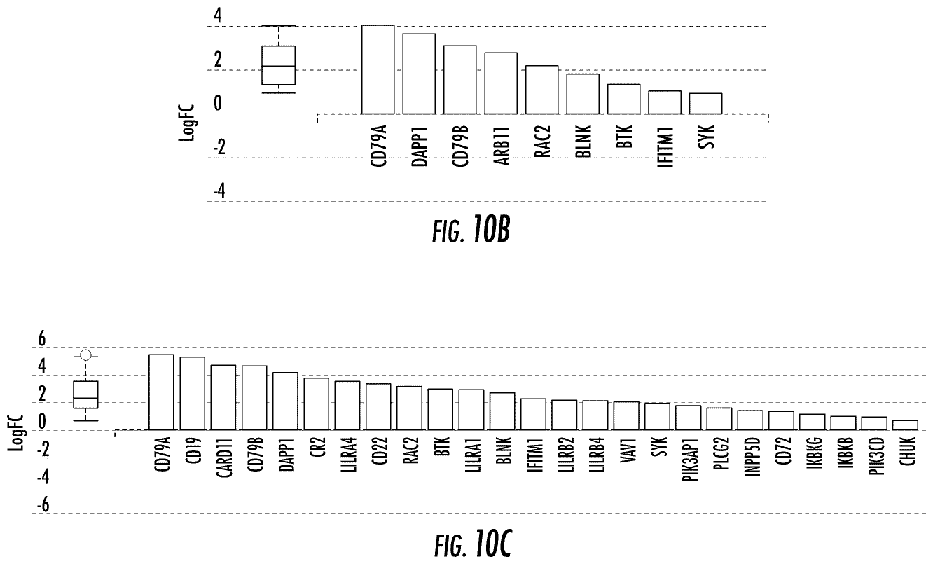

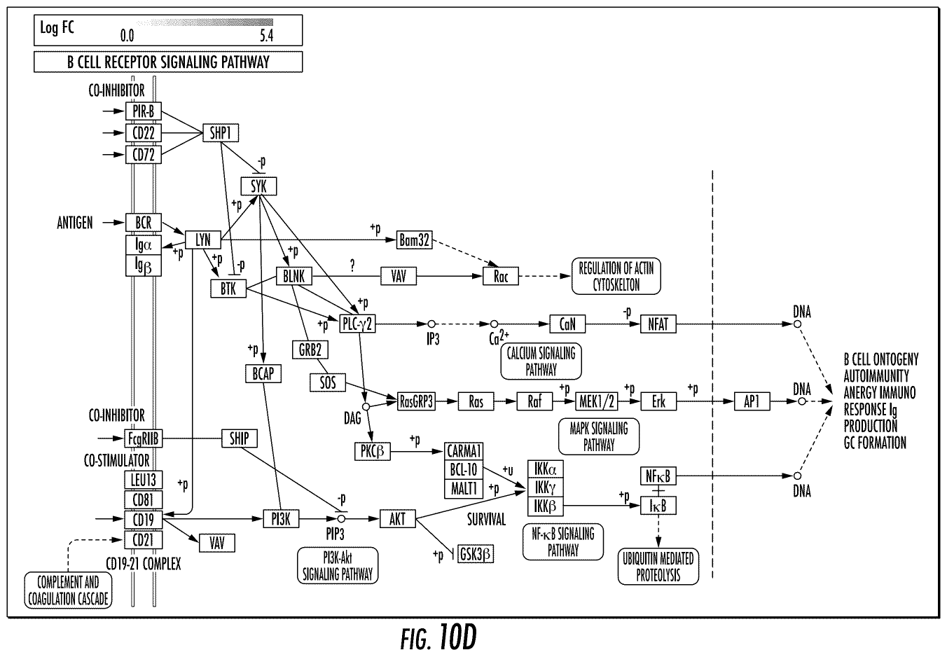

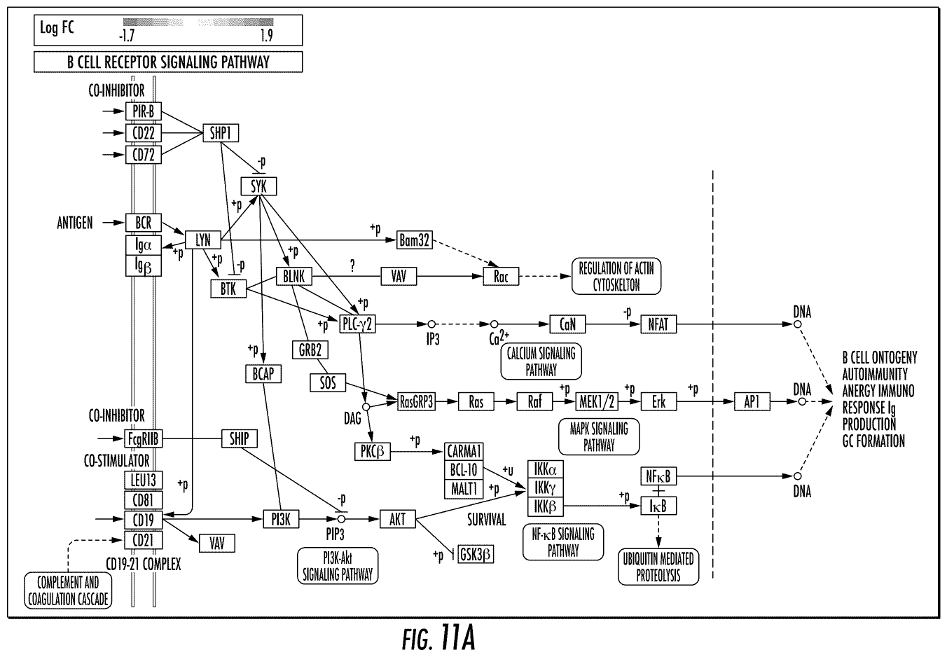

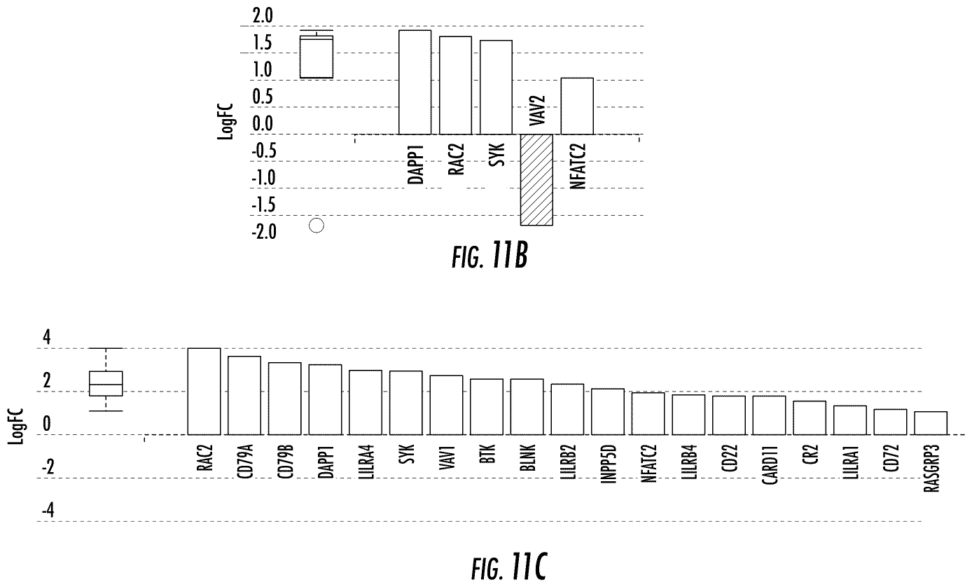

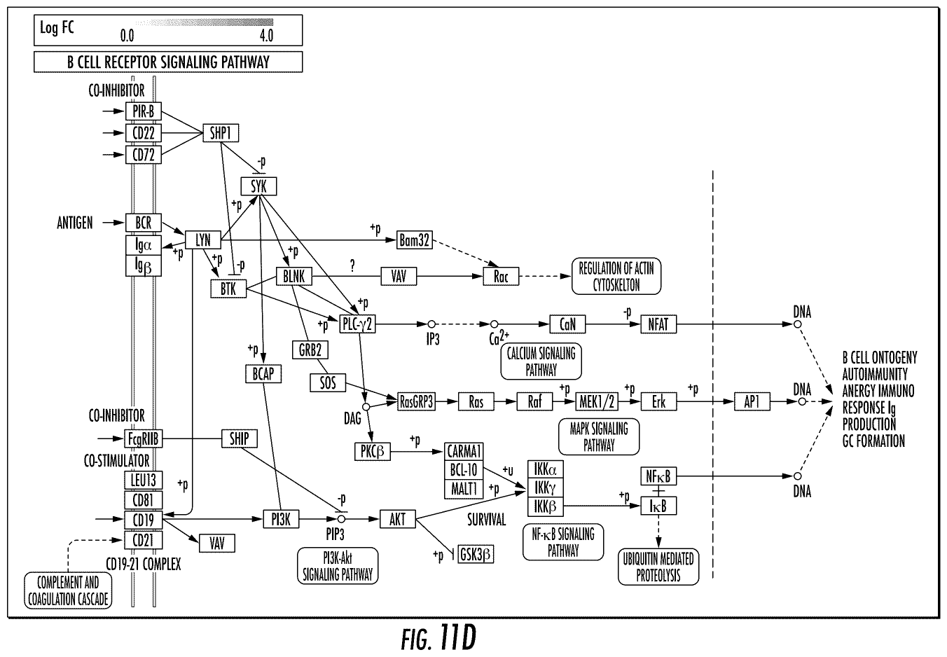

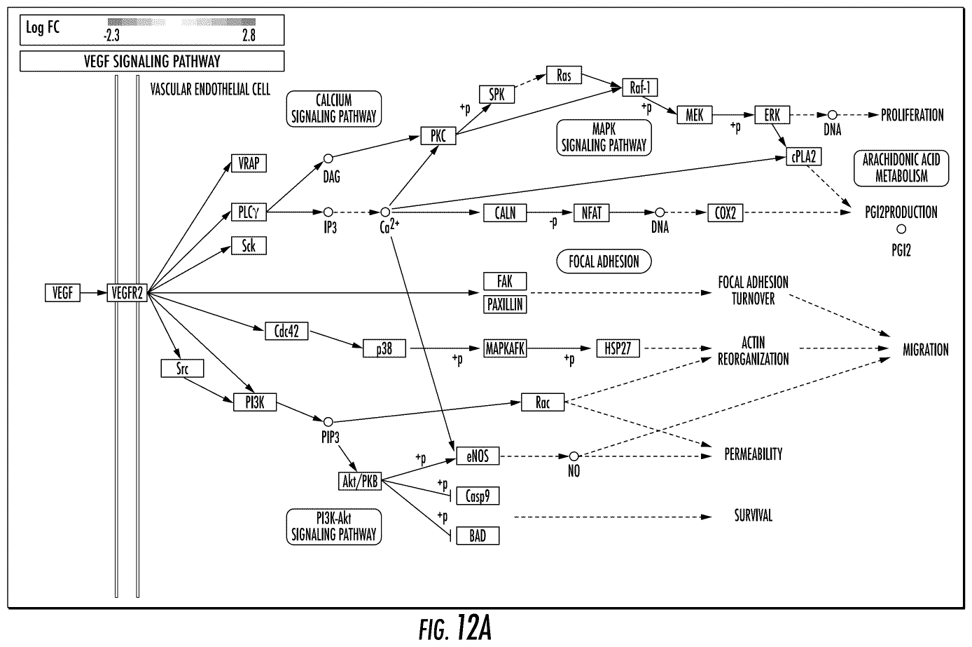

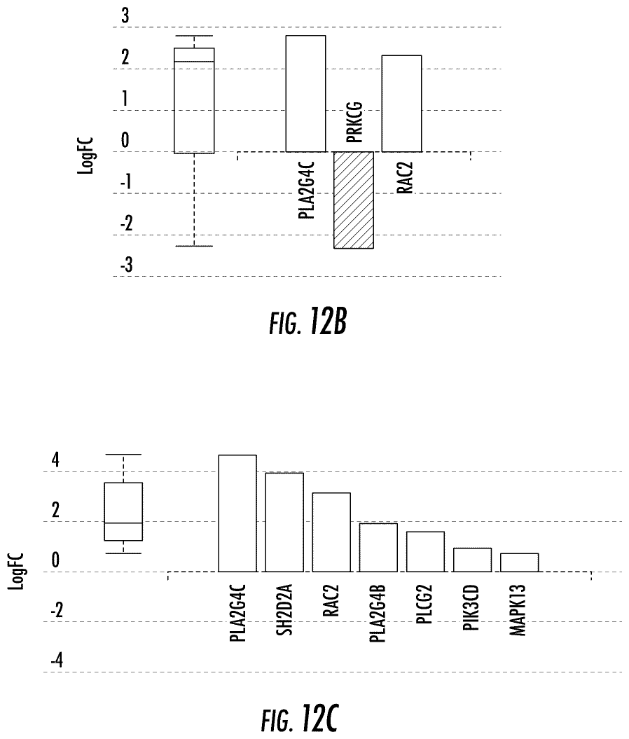

FIGS. 10A, 10B, 10C, 10D, 11A, 11B, 11C, and 11D show results identifying signatures that have overlapping genes expressed in B cell receptor signaling pathway including B cell signature.

As shown by the pathway diagram of FIG. 10A and the plot of FIG. 10B, the following B cell receptor signaling pathway genes were identified as DEGs in ciliary body-iris tissue in response to the 4E11 vg/eye dose of Ixo-vec: CD79A, DAPP1, CD79B, CARD11, RAC2, BLNK, BTK, IFITIvII, and SYK. As shown by the plot of FIG. 10C and the pathway diagram of FIG. 10D, the following B cell receptor signaling pathway genes were identified as DEGs in ciliary body-iris tissue in response to the 2E13 vg/eye dose of Ixo-vec: CD79A, CD19, CARD11, CD79B, DAPP1, CR2, LILRA4, CD22, RAC2, BTK, LILRA1, BLNK, IFITM1, LILRB2, LILRB4, VAV1, SYK, PIK3AP1, PLCG2, INPP5D, CD72, IKBKG, IKBKB, PIK3CD, and CHUK.

As shown by the pathway diagram of FIG. 11A and the plot of FIG. 11B, the following B cell receptor signaling pathway genes were identified as DEGs in retina tissue in response to the 4E11 vg/eye dose of Ixo-vec: DAPP1, RAC2, SYK, VAV2, and NFATC2. As shown by the plot of FIG. 11C and the pathway diagram of FIG. 11D, the following B cell receptor signaling pathway genes were identified as DEGs in retina tissue in response to the 2E13 vg/eye dose of Ixo-vec: RAC2, CD79A, CD79B, DAPP1, LILRA4, SYK, VAV1, BTK, BLNK, LILRB2, INPP5D, NFATC2, LILRB4, CD22, CARD11, CR2, LILRA1, CD72 and RASGRP3.

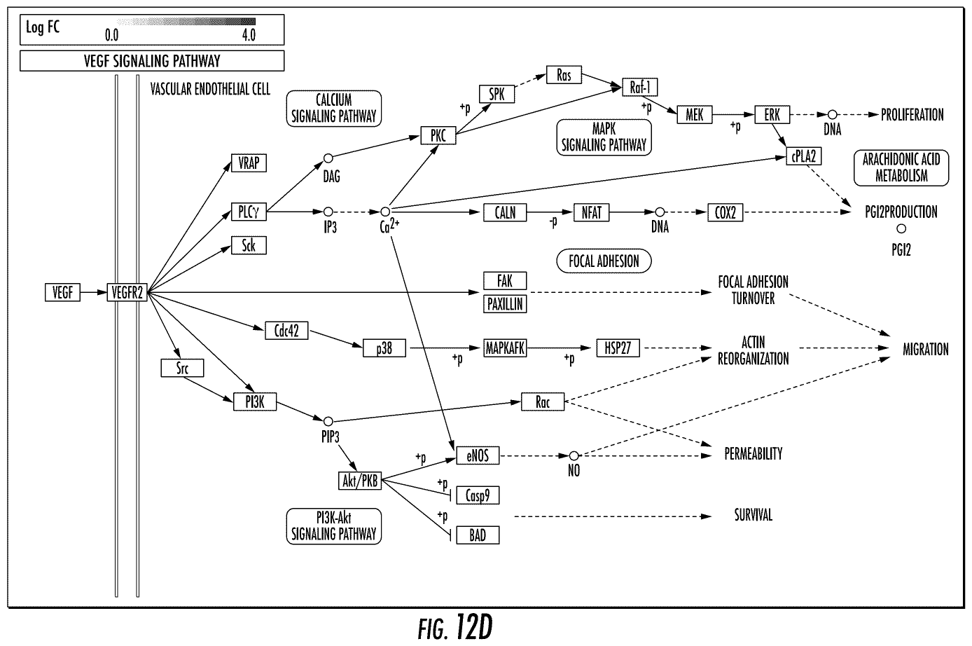

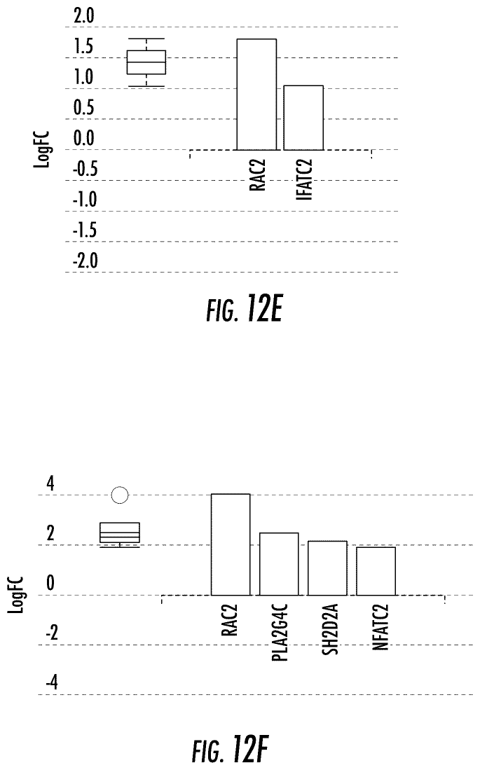

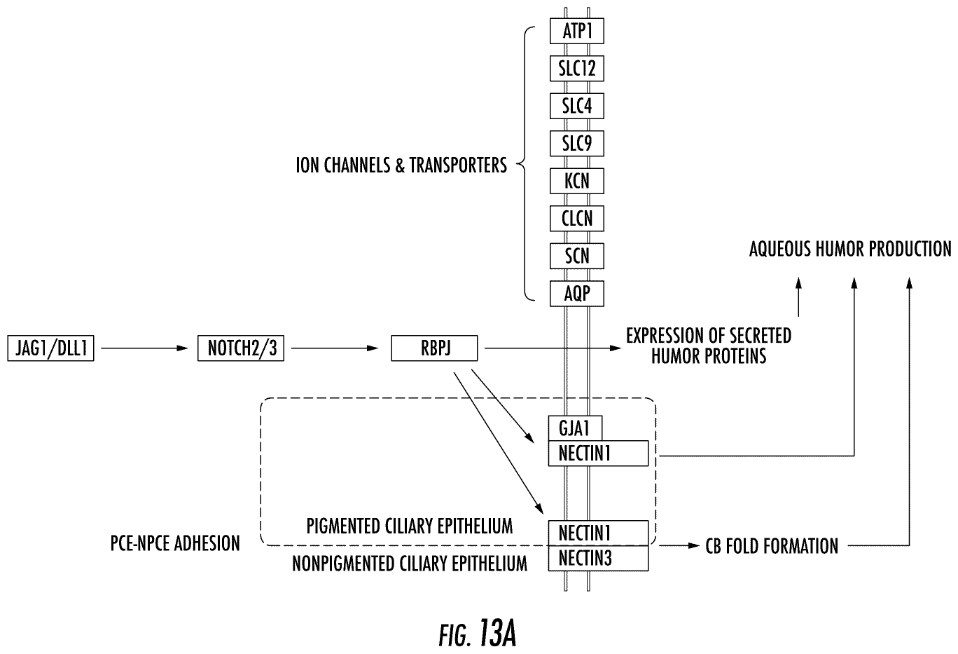

FIGS. 12A, 12B, 12C, 12D, 12E, and 12F show results identifying genes differentially expressed in VEGF signaling pathway. As shown by the pathway diagram of FIG. 12A and the plots of FIGS. 12B and 12C, VEGF signaling pathway genes were identified as DEGs in ciliary body-iris tissue at two dosages of IVT administered Ixo-vec. FIG. 12A depicts a schematic diagram of the VEGF signaling pathway. As shown by the plot of FIG. 12B, the following VEGF signaling pathway genes were identified as DEGs in ciliary body-iris tissue in response to the 4E11 vg/eye dose of Ixo-vec: PLA2G4C, PRKCG, and RAC2. As shown by the plot of FIG. 12C, the following VEGF signaling pathway genes were identified as DEGs in ciliary body-iris tissue in response to the 2E13 vg/eye dose of Ixo-vec: PLA2G4C, SH2D2A, RAC2, PLA2G4B, PLCG2, PIK3CD, and MAPK13. As shown by the pathway diagram of FIG. 12D and the plot of FIG. 12F, the following pathway genes were identified as DEGs in retina tissue in response to the 2E13 vg/eye dose of Ixo-vec: RAC2, PLA2G4C, SH2D2A, and NFATC2. As shown by the plot of FIG. 12E, the following VEGF signaling pathway genes were identified as DEGs in retina tissue in response to the 4E11 vg/eye dose of Ixo-vec: RAC2 AND NFATC2. These results show that VEGF signaling is not activated in ocular tissues across doses for all tissues examined. Thus, Ixo-vec is unlikely to exacerbate VEGF-mediated pathology of wet AMD.

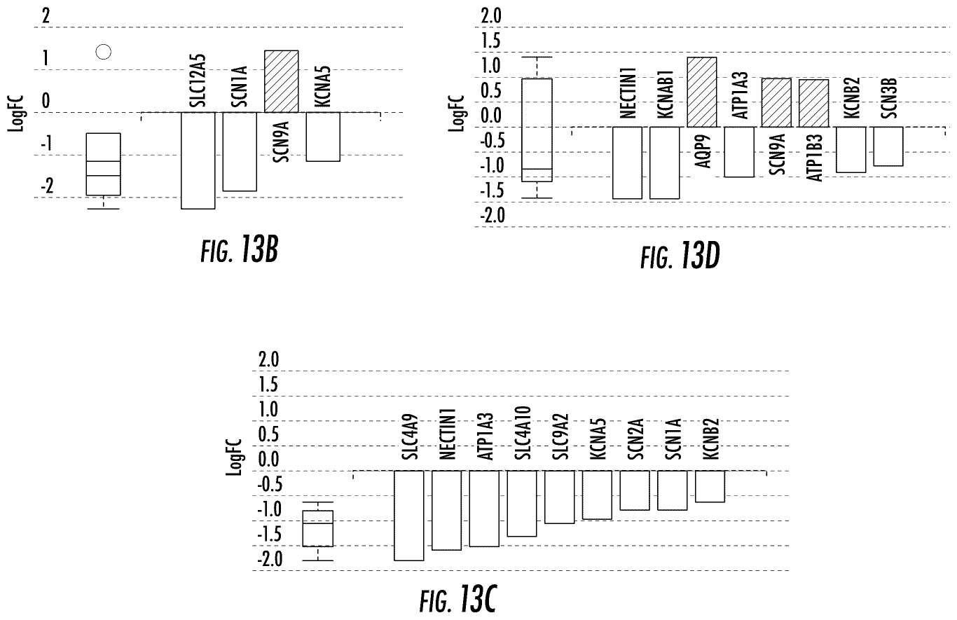

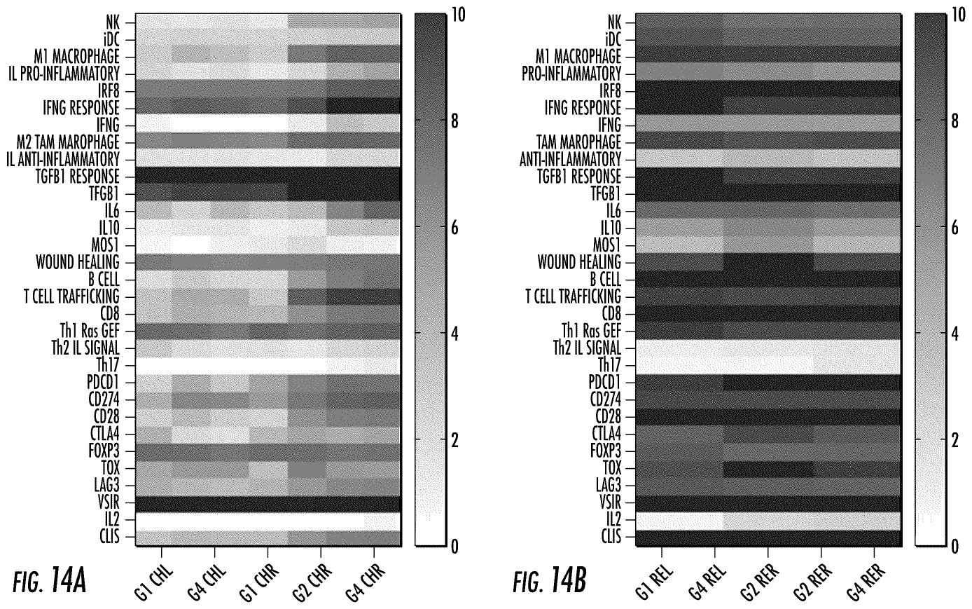

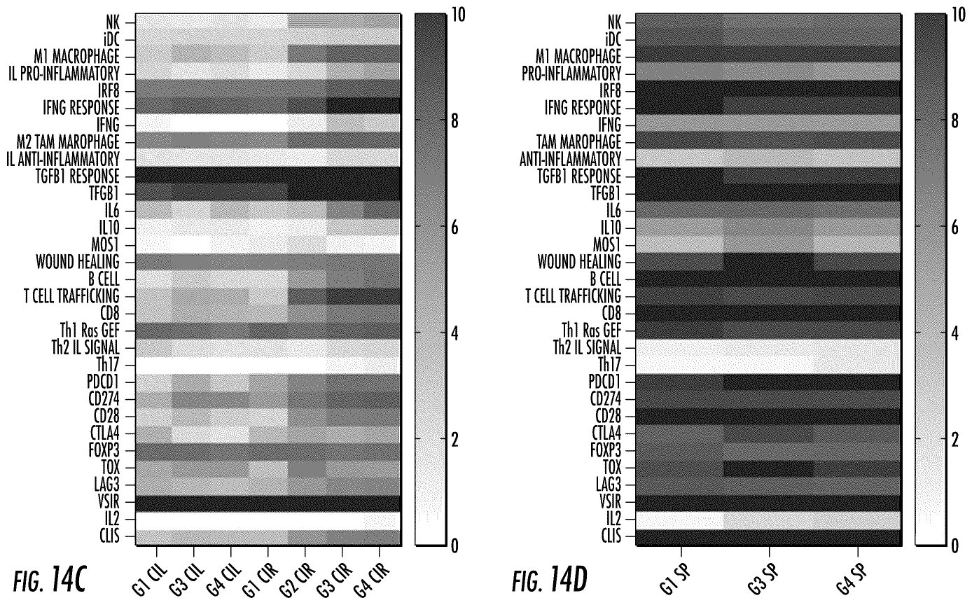

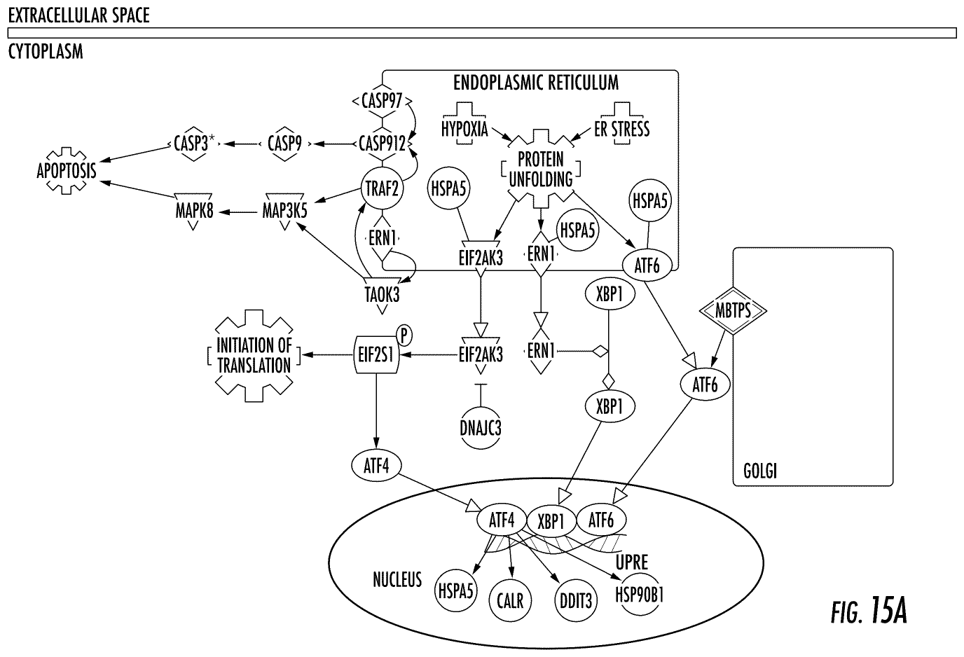

FIGS. 13A, 13B, 13C, and FIG. 13D show results identifying genes differentially expressed in the aqueous humor production and ciliary body fold formation pathway in ciliary body-iris tissue at three dosages of IVT administered Ixo-vec. As shown by the plot of FIG. 13B, the following aqueous humor production and ciliary body fold formation pathway genes were identified as DEGs in ciliary body-iris tissue in response to the 4E11 vg/eye dose of Ixo-vec: SLC12A5, SCN1A, SCN9A, and KCNA5. As shown by the plot of FIG. 13C, the following aqueous humor production and ciliary body fold formation pathway genes were identified as DEGs in ciliary body-iris tissue in response to the 2E12 vg/eye dose of Ixo-vec: SLC4A9, NECTIN1, ATP1A3, SLC4A10, SLC9A2, KCNA5, SCN2A, SCN1A, and KCNB2. As shown by the plot of FIG. 13D, the following aqueous humor production and ciliary body fold formation pathway genes were identified as DEGs in ciliary body-iris tissue in response to the 2E13 vg/eye dose of Ixo-vec: NECTIN1, KCNAB1, AQP9, ATP1A3, SCN9A, ATP1B3, KCNB2, and SCN3B.

FIGS. 14A-14D show heat maps of composite scores for activation of differentially expressed genes (“DEGs”) from choroid (FIG. 14A), retina (FIG. 14B), ciliary body-iris (FIG. 14C), and spleen (FIG. 14D) tissue samples of the Ixo-vec dose-response analysis described in this Example 1. These results add further confirmation that the immunological processes identified by the pathway analyses above (e.g., M1 macrophage signature).

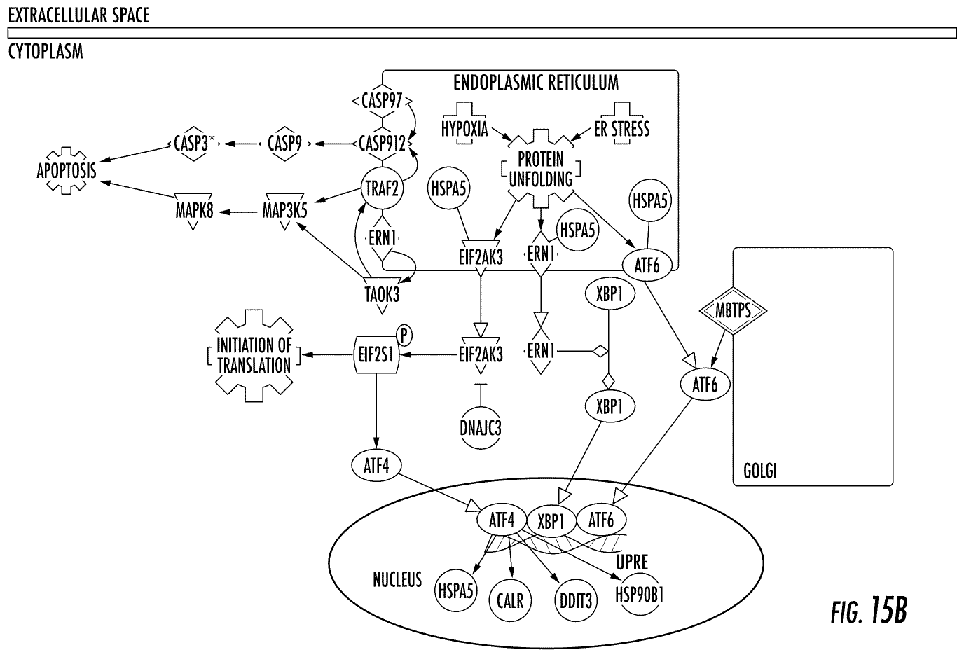

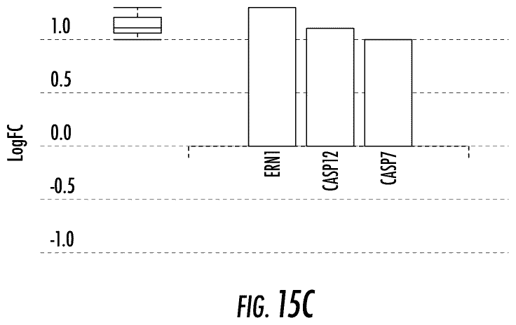

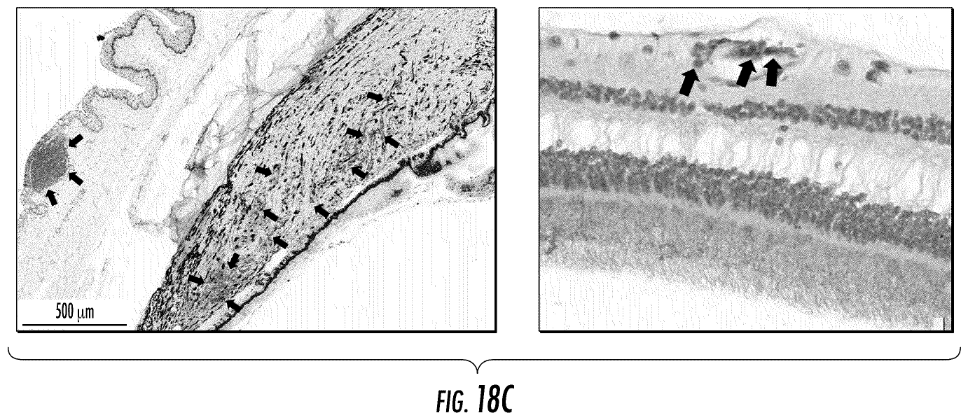

FIGS. 15A-17B show results identifying genes differentially expressed in the endoplasmic reticulum stress pathway in response in ciliary body-iris, retina, and choroid tissues at two dosages of IVT administered Ixo-vec. For example, the results depicted in FIG. 17B indicate that the genes CAP12 and XBP1 are differentially expressed in ciliary body-iris tissue between sham-injected eyes and eyes dosed at 2E13 vg/eye.

FIGS. 18A-18D show representative immunohistochemistry (IHC) staining for B lymphocyte markers in the anterior tissues of the eye. No CD20+ cell (left image) or CD138+ cells (right image) were observed in the vehicle (sham injected) eyes (FIG. 18A). Cells expressing CD20 were detected in the low dose group (FIG. 18B). Plasma cell marker CD138+ was only detected in the high dose group (FIG. 18C).

FIGS. 19A-19C show representative immunohistochemistry (IHC) staining for T lymphocyte markers in the anterior tissues of the eye. Vehicle (sham injected) eyes had no detectable expression of either CD4+ or CD8+ T cells (FIG. 19A). Both CD4+ and CD8+ T cells were observed at doses of 4E11 vg/eye and higher (FIGS. 19B-19C).

FIG. 20 shows change in expression levels of sentinel genes that represent a portion of the genes associated with TLR signaling (top), complement activation (middle), and B cell receptor signaling (bottom) pathways in the ciliary body/iris tissues across dosed groups, relative to the sham-injected group.

Sentinel genes were chosen to represent pathways identified by RNA-seq analysis. FIG. 21 shows a heatmap of RT-qPCR of these sentinel genes from biological pathways and processes evaluated in ciliary body/iris tissues.

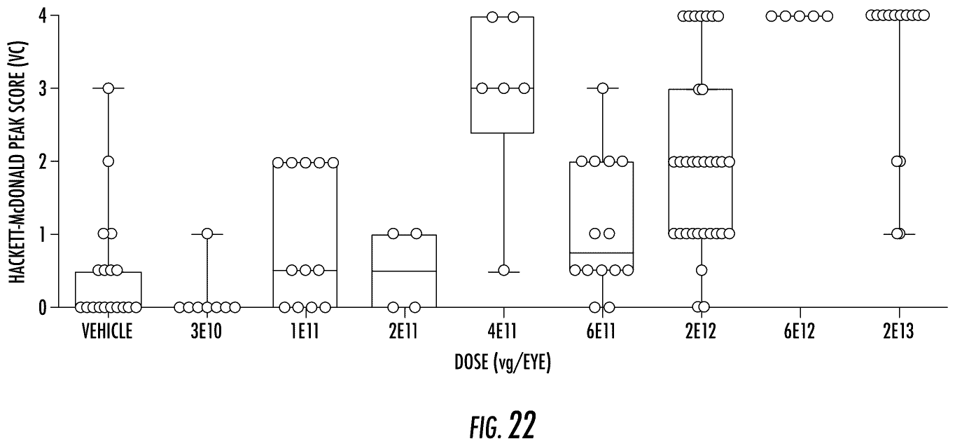

FIG. 22 shows vitreous inflammation graded on the level of vitreous cells observed in non-human primates at the indicated doses of Ixo-vec. Vitreous cell grading is reported as peak score using Hackett-McDonald method.

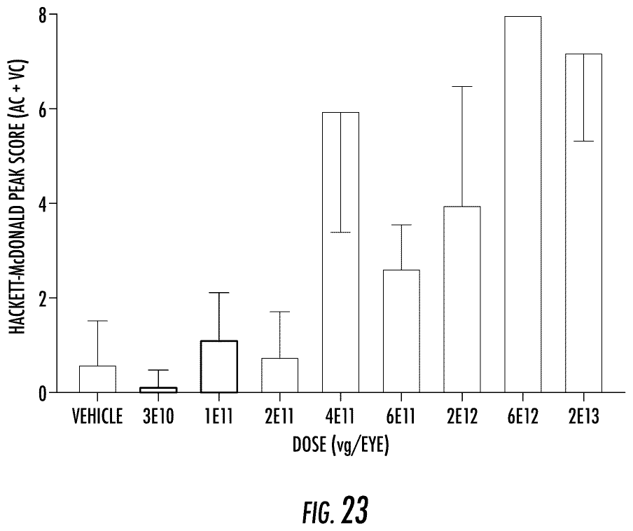

FIG. 23 shows the levels of anterior chamber and vitreous chamber cell inflammation measured in non-human primates with the indicated doses of Ixo-vec. Anterior cell (AC) and vitreous cell (VC) grading is reported as peak score using the Hackett-McDonald method. The maximum score for each chamber is 4. For each eye, the scores of both AC and VC were summated. For each group, the cumulative AC and VC scores are reported as mean with standard deviation.

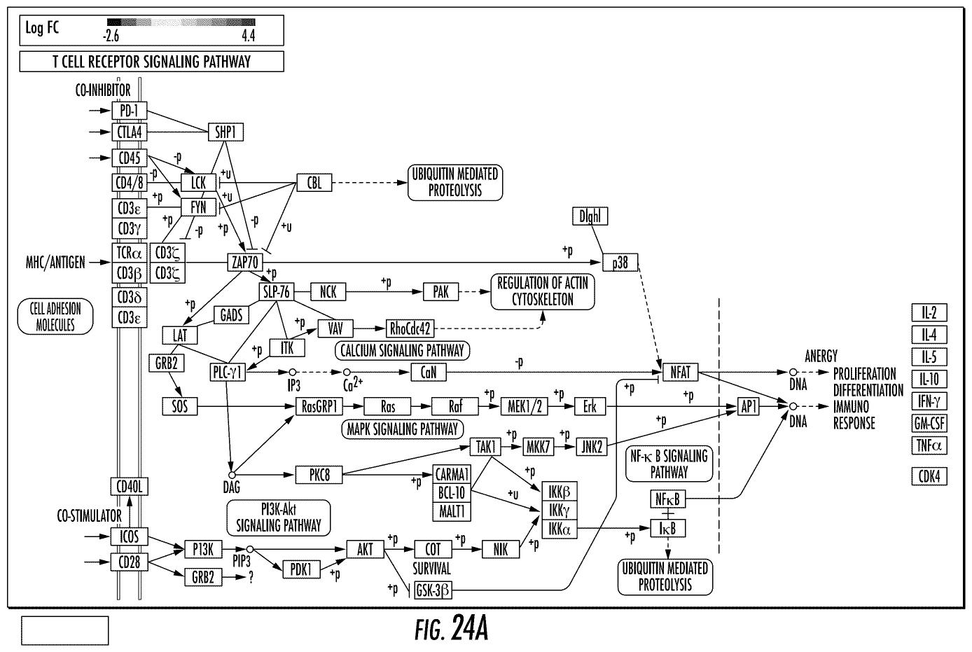

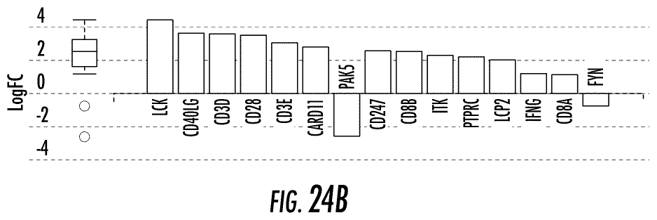

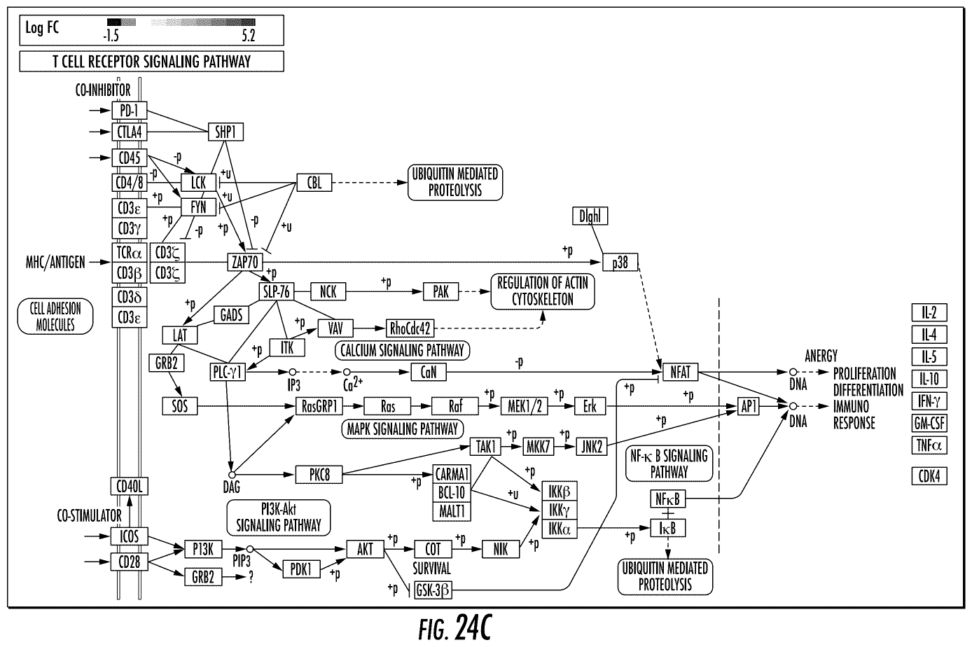

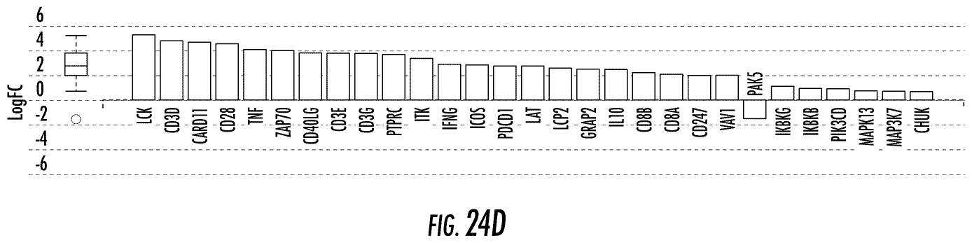

FIGS. 24A, 24B, 24C, and 24D show results identifying genes differentially expressed in T cell receptor signaling pathway. As shown by the pathway diagram of FIG. 24A and the plot of FIG. 24B, the following T cell receptor signaling pathway genes were identified as DEGs in ciliary body-iris tissue in response to the 4E11 vg/eye dose of Ixo-vec: LCK, CD40LG, CD3D, CD28, CD3E, CARD11, PAK5, CD247, CD8B, ITK, PTPRC, LCP2, IFNG, CD8A, AND FYN. As shown by the pathway diagram of FIG. 24C and the plot of FIG. 24D, the following T cell receptor signaling pathway genes were identified as DEGs in ciliary body-iris tissue in response to the 2E13 vg/eye dose of Ixo-vec: LCK, CD3D, CARD11, CD28, TNF, ZAP70, CD40LG, CD3E, CD3G, PTPRC, ITK, IFNG, ICOS, PDCD1, LAT, LCP2, GRAP2, IL10, CD8B, CD8A, CD247, VAV1, PAK5, IKBKG, IKBKB, PIK3CD, MAPK13, MAP3K7, AND CHUK.

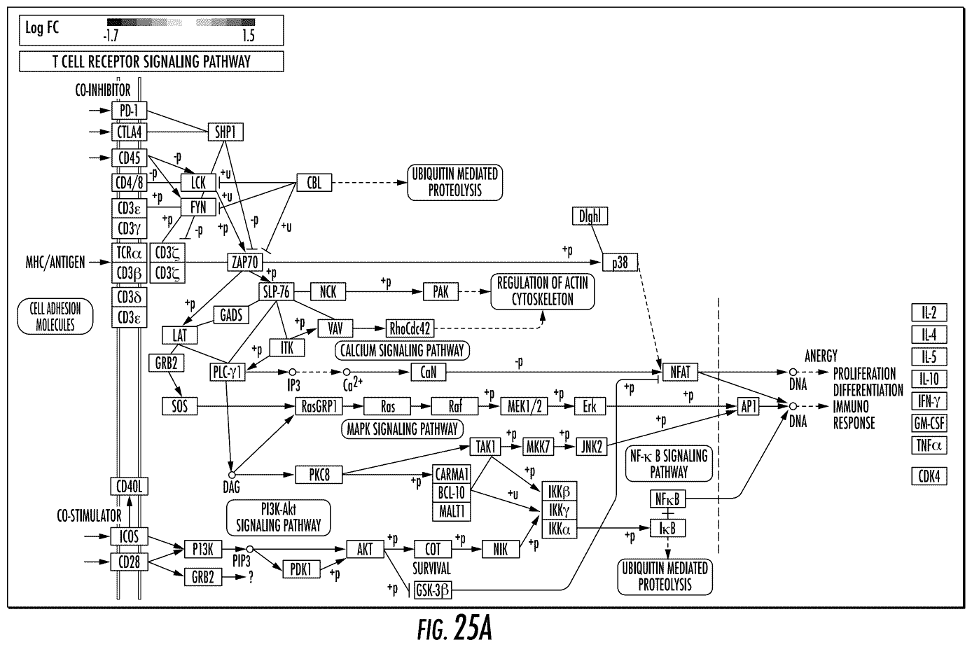

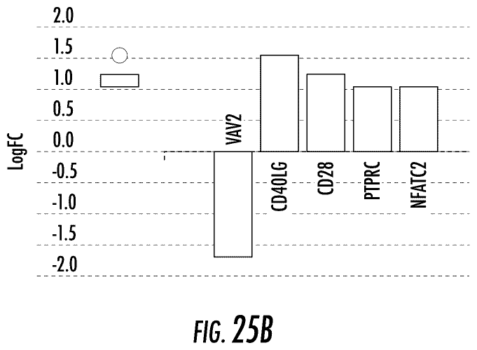

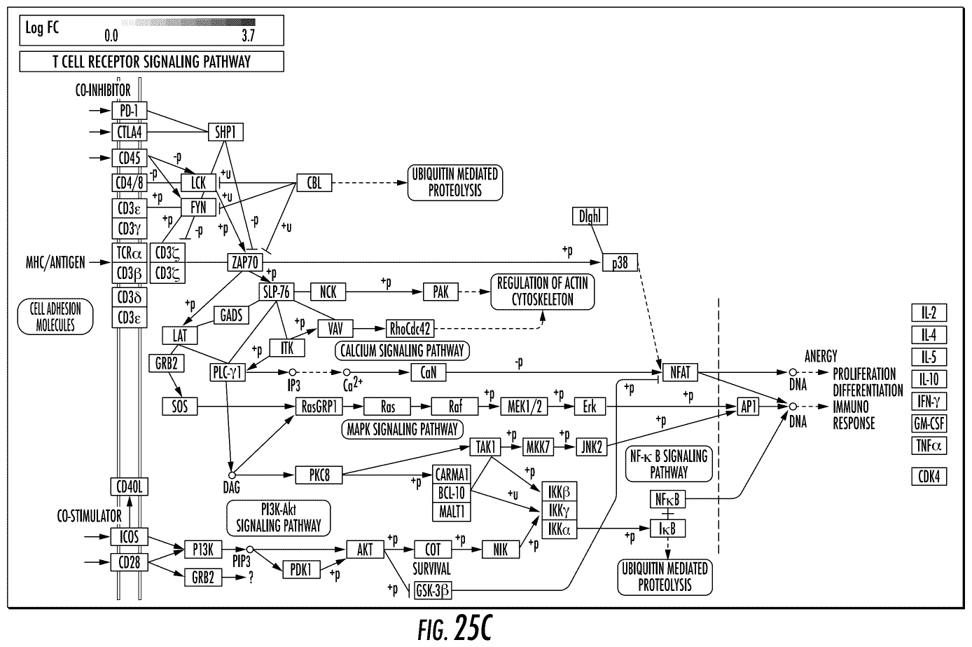

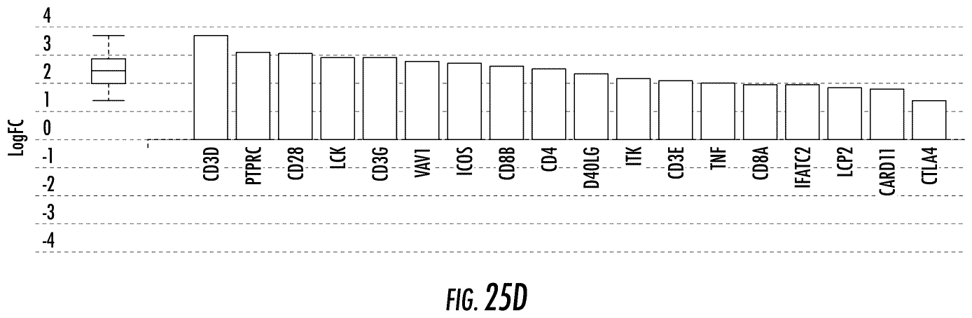

FIGS. 25A, 25B, 25C, and 25D show results identifying genes differentially expressed in T cell receptor signaling pathway. As shown by the pathway diagram of FIG. 25A and the plot of FIG. 25B, the following T cell receptor signaling pathway genes were identified as DEGs in retina tissue in response to the 4E11 vg/eye dose of Ixo-vec: VAV2, CD40LG, CD28, PTPRC, and NFATC2. As shown by the pathway diagram of FIG. 25C and the plot of FIG. 25D, the following T cell receptor signaling pathway genes were identified as DEGs in retina tissue in response to the 2E13 vg/eye dose of Ixo-vec: CD3D, PTPRC, CD28, LCK, CD3G, VAV1, ICOS, CD8B, CD4, CD40LG, ITK, CD3E, TNF, CD8A, NFATC2, LCP2, CARD11, and CTLA4.

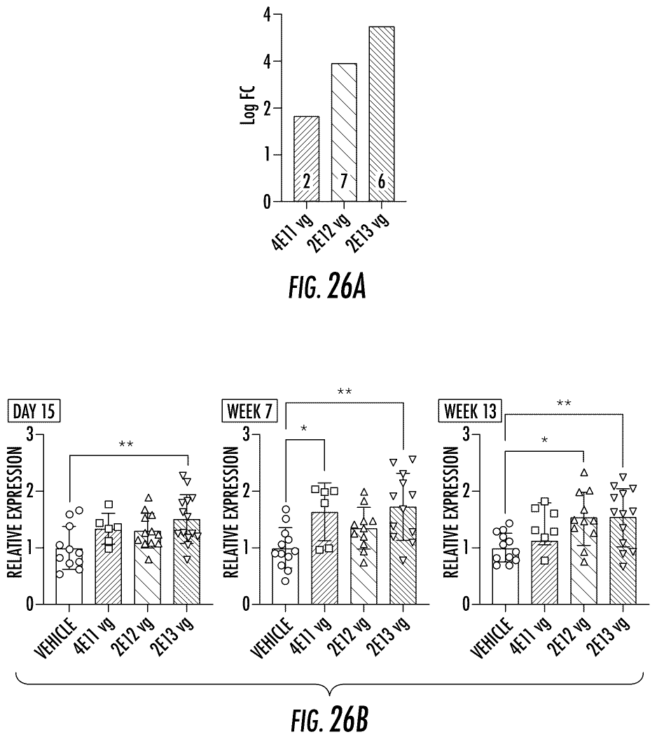

FIGS. 26A and 26B show expression levels of TLR10 in response to IVT administered Ixo-vec. FIG. 26A shows TLR10 expression levels in ciliary body-iris tissue in response to doses of 4E11 vg/eye and higher. FIG. 26B shows TRL10 expression levels in whole blood at Day 15, Week 7, and Week 13 after Ixo-vec dosage, or sham-injection of vehicle eyes.

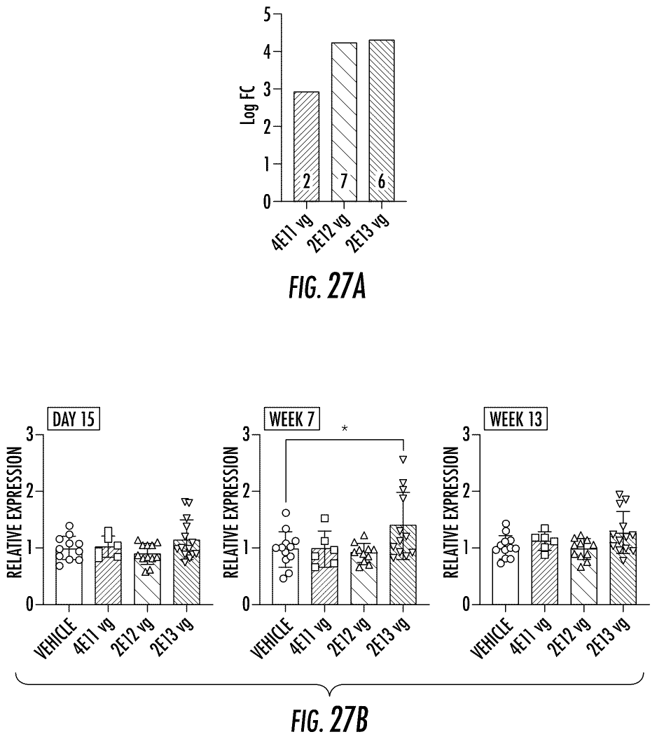

FIGS. 27A and 27B show expression levels of CXCR3 in response to IVT administered Ixo-vec. FIG. 27A shows CXCR3 expression levels in ciliary body-iris tissue in response to doses of 4E11 vg/eye and higher. FIG. 27B shows CXCR3 expression levels in whole blood at Day 15, Week 7, and Week 13 after Ixo-vec dosage, or sham-injection of vehicle eyes.

While the foregoing disclosure of the present invention has been described in some detail by way of example and illustration for purposes of clarity and understanding, this disclosure including the examples, descriptions, and embodiments described herein are for illustrative purposes, are intended to be exemplary, and should not be construed as limiting the present disclosure. It will be clear to one skilled in the art that various modifications or changes to the examples, descriptions, and embodiments described herein can be made and are to be included within the spirit and purview of this disclosure and the appended claims. Further, one of skill in the art will recognize a number of equivalent methods and procedure to those described herein. All such equivalents are to be understood to be within the scope of the present disclosure and are covered by the appended claims.

What is claimed is:

1. A method of screening a patient prior to intravitreal rAAV therapy to identify a patient at risk of AAV-induced inflammation, the method comprising: (a) obtaining a biological sample from the patient; (b) detecting expression levels of each of a set of genes in the biological sample; (c) determining whether the detected expression levels indicate an increased likelihood of AAV-induced inflammation by comparison to a predetermined threshold expression levels for the set of genes; and (d) identifying the patient as in need of anti-inflammatory treatment if an increased likelihood of AAV-induced inflammation is determined.

2. A method of monitoring a patient who has received or is receiving an rAAV treatment and who also is receiving an anti-inflammatory treatment for AAV-induced inflammation, the method comprising: (a) obtaining a biological sample from the patient; (b) detecting expression levels of each of a set of genes in the biological sample; (c) determining whether the detected expression levels indicate a reduced likelihood of AAV-induced inflammation by comparison to a predetermined threshold expression levels for the set of genes; and (d) identifying the patient as a candidate for reduced anti-inflammatory treatment if a reduced likelihood of AAV-induced inflammation is determined.

3. A method of identifying a patient in need of an anti-inflammatory treatment from a population of patients who have received intravitreal rAAV therapy, the method comprising: (a) obtaining a biological sample from the patient who has received an rAAV treatment; (b) detecting expression levels of each of a set of genes in the biological sample; (c) determining whether the detected expression levels indicate an increased likelihood of AAV-induced inflammation by comparison to a predetermined threshold expression levels for the set of genes; and (d) identifying the patient as in need of anti-inflammatory treatment if an increased likelihood of AAV-induced inflammation is determined.

4. A method of selecting and treating a patient who has an increased likelihood of AAV-induced inflammation from a population of patients who have received an rAAV treatment, the method comprising: (a) obtaining a biological sample from the patient who has received an rAAV treatment; (b) detecting expression levels of each of a set of genes in the biological sample; (c) determining whether the detected expression levels indicate an increased likelihood of AAV-induced inflammation by comparison to a predetermined threshold expression levels for the set of genes; and (d) administering an anti-inflammatory agent to the patient if an increased likelihood of AAV-induced inflammation is determined.

5. A method of providing immunosuppressive treatment in a patient who has received intravitreal rAAV therapy, the method comprising: (a) obtaining a biological sample from the patient who has received an rAAV treatment; (b) detecting expression levels of each of a set of genes in the biological sample; (c) determining whether the detected expression levels indicate an increased likelihood of AAV-induced inflammation by comparison to predetermined threshold levels for the genes; and (d) administering an immunosuppressive treatment to the patient if an increased likelihood of AAV-induced inflammation is determined.

6. The method of any one of claims 1-5, wherein the set of genes comprises at least two genes selected from: A2M, B2M, BLNK, BTK, C1QA, C1QB, C1QC, C1R, C1S, C2, C3, C4B, CARD11, CASP8, CCL3, CCL4L2, CCL5, CD14, CD19, CD22, CD247, CD28, CD3D, CD3E, CD3G, CD4, CD40, CD40LG, CD72, CD74, CD79A, CD79B, CD86, CD8A, CD8B, CFHR4, CHUK, CIITA, CPB2, CR1, CR2, CTLA4, CTSS, CXCL10, CXCL11, CXCL8, CXCL9, CXCR3, DAPP1, F12, F13A1, F2RL3, FCGR1A, FCGR2A, FCGR3A, FYN, GRAP2, HCK, HLA-DMA, HLA-DMB, HLA-DOA, HLA-DOB, HLA-DPA1, HLA-DPB1, HLA-DQA1, HLA-DRA, HLA-DRB1, HLA-E, HLA-F, HLA-G, HSP90AA1, ICOS, IFI30, IFITIvII, IFITM1, IFNG, IKBKB, IKBKE, IKBKG, IL10, IL12A, IL12B, IL1B, IL6, INPP5D, IRF7, ITGAM, ITGAX, ITGB2, ITK, IY96, KLRC2, KLRD1, KPK3CD, LAT, LCK, LCP2, LGMN, LILRA1, LILRA4, LILRB2, LILRB4, LR2, LY97, MAP3K7, MAPK13, MP3K7, MYD88, NCF1, NFATC2, PAK5, PDCD1, PIK3AP1, PIK3CD, PLA2G4B, PLA2G4C, PLAU, PLCG2, PLD1, PRKCD, PROC, PSME1, PTPRC, RAC2, RASGRP3, SCIN, SERPINA1, SERPINA5, SERPINE1, STAT1, SYK, TAP1, TAP2, TLR1, TLR4, TLR7, TLR8, TLR10, TNF, VASP, VAV1, VAV2, VSIG4, WAS, WAV1, and ZAP70.

7. The method of any one of claims 1-6, wherein the set of genes is selected from:
(a) CXCL9, CXCL10, CXCL11, CCL5 and CCL3;
(b) CXCL9, CXCL11, CXCL10, IL6, TNF, CCL5, CXCL8, CCL3, TLR8, CCL4L2, IY96, IL12A, IL12B, STAT1, IKBKE, IL1B, TLR1, CASP8, IKBKG, TLR4, IRF7, CD14, MYD88, CD86, IKBKB, KPK3CD, MP3K7, CHUK, and CD40;
(c) CXCL10, CXCL9, CCL5, CCL4L2, and MYD88;
(d) CXCL10, CXCL9, CCL5, CXCL11, IL1B, TLR8, CCL4L2, CD86, TLR2, STAT1, TNF, LY97, TLR4, IRF7, TLR7 and IKBKE;
(e) PLA2G4C, PRKCG, RAC2, PTPRC, FCGR3A, FCGR2A, SYK, and FCGR1A;
(f) PLA2G4C, PTPRC, RAC2, LAT, NCF1, FCGR3A, WAV1, SYK, PLA2G4B, WAS, PLCG2, HCK, FCGR2A, INPP5D, FCGR2A, INPP5D, FCGR1A, PIK3CD, PRKCD, and VASP;
(g) RAC2, SYK, VAV2, FCGR3A and PTPRC;
(h) RAC2, FCGR2A, PTPRC, FCGR3A, SYK, VAV1, NCF1, PLA2G4C, WAS, SCIN, FCGR1A, INPP5D, HCK and PLD1;
(i) C1R, ITGAM, C4B, F13A1, ITGB2 and C1QC;
(j) SERPINA5, CR1, CR2, C4B, ITGAX, PROC, ITGAM, C3, F12, F13A1, ITGB2, SERPINE1, PLAU, C1S, C1QC, CFHR4, C1QA and C1R;
(k) ITGAX, FGG and CPB2;
(l) F13A1, ITGAX, C1QB, ITGAM, C1QC, C1QA, C2, ITGB2, C3, C4B, C1S, SERPINA1, VSIG4, SERPINA5, CR2, A2M, and F2RL3;
(m) HLA-DOB, KLRC2, CD8B, TAP1, HLA-DRA, CTSS, HLA-DMB, IFNG, CD8A and LGMN;
(n) HLA-DOB, KLRD1, TNF, IFNG, TAP1, HLA-G, CIITA, KLRC2, CD8B, TAP2, HLA-E, CD8A, CTSS, HLA-DPB1, HLA-DPA1, HLA-DRB1, HLA-DMB, HLA-DRA, HLA-DOA, HLA-F, CD74, HLA-DMA, IFI30, PSME1, HSP90AA1, and LGMN;
(o) HLA-DRB1, HLA-DQA1, HLA-DPA1, and KLRD1;
(p) HLA-DRA, HLA-G, B2M, HLA-DQA1, HLA-DRB1, HLA-DPB1, CIITA, CD74, HLA-DOA, HLA-DMB, HLA-DMA, HLA-E, CD8B, HLA-DPA1, CD4, TAP1, IFI30, KLRD1, HLA-DOB, TNF, CD8A, TAP2, and PSME1;
(q) CD79A, DAPP1, CD79B, CARD11, RAC2, BLNK, BTK, IFITIvII, and SYK;
(r) CD79A, CD19, CARD11, CD79B, DAPP1, CR2, LILRA4, CD22, RAC2, BTK, LILRA1, BLNK, IFITM1, LILRB2, LILRB4, VAV1, SYK, PIK3AP1, PLCG2, INPP5D, CD72, IKBKG, IKBKB, PIK3CD, and CHUK;
(s) DAPP1, RAC2, SYK, VAV2, and NFATC2;
(t) RAC2, CD79A, CD79B, DAPP1, LILRA4, SYK, VAV1, BTK, BLNK, LILRB2, INPP5D, NFATC2, LILRB4, CD22, CARD11, CR2, LILRA1, CD72 and RASGRP3;
(u) LCK, CD40LG, CD3D, CD28, CD3E, CARD11, PAK5, CD247, CD8B, ITK, PTPRC, LCP2, IFNG, CD8A, and FYN;
(v) LCK, CD3D, CARD11, CD28, TNF, ZAP70, CD40LG, CD3E, CD3G, PTPRC, ITK, IFNG, ICOS, PDCD1, LAT, LCP2, GRAP2, IL10, CD8B, CD8A, CD247, VAV1, PAK5, IKBKG, IKBKB, PIK3CD, MAPK13, MAP3K7, and CHUK;
(w) VAV2, CD40LG, CD28, PTPRC, and NFATC2; and
(x) CD3D, PTPRC, CD28, LCK, CD3G, VAV1, ICOS, CD8B, CD4, CD40LG, ITK, CD3E, TNF, CD8A, NFATC2, LCP2, CARD11, and CTLA4.

8. The method of any one of claims 1-7, wherein the biological sample comprises a first biological sample obtained at a first time point and a second biological sample obtained at a second time point.

9. The method of any one of claims 1-8, wherein detecting the expression levels comprises sequencing RNA in the biological sample.

10. The method of any one of claims 1-9, wherein the threshold expression level is a log 2 fold-change in expression>10.61.

11. The method of any one of claims 1-10, wherein the AAV-induced inflammation is ocular inflammation.

12. The method of any one of claims 1-11, wherein the biological sample comprises ocular cells.

13. The method of any one of claims 1-12, wherein the biological sample comprises blood, serum, tears, or a combination thereof.

14. The method of any one of claims 1-13, wherein the rAAV treatment was administered to an eye of the subject.

15. The method of claim 14, wherein the rAAV treatment was administered to the contralateral eye of the patient.

16. The method of any one of claims 4-13, wherein the anti-inflammatory agent is administered to the eye.

17. The method of any one of claims 4-15, wherein the anti-inflammatory agent is a steroid; optionally wherein the steroid is selected from a corticosteroid, prednisone, and difluprednate.

18. The method of any one of claims 16-17, wherein the steroid is administered systemically.

19. The method of any one of claims 16-17, wherein the steroid is administered orally.

20. The method of any one of claims 16-17, wherein the steroid is administered topically.

21. The method of any one of claims 1-20, wherein the AAV-induced inflammation is in the ciliary body and/or in the iris.

22. The method of any one of claims 1-20, wherein the AAV-induced inflammation is in the retina.

23. The method of any one of claims 1-20, wherein the AAV-induced inflammation is in the choroid.

24. The method of claim 11, wherein the expression levels of the set of genes in the biological sample relative to the predetermined threshold levels indicates a likelihood of AAV-induced inflammation in the ciliary body and/or the iris, and wherein the set of genes is selected from:
(a) CXCL9, CXCL10, CXCL11, CCL5 and CCL3;
(b) CXCL9, CXCL11, CXCL10, IL6, TNF, CCL5, CXCL8, CCL3, TLR8, CCL4L2, IY96, IL12A, IL12B, STAT1, IKBKE, IL1B, TLR1, CASP8, IKBKG, TLR4, IRF7, CD14, MYD88, CD86, IKBKB, KPK3CD, MP3K7, CHUK, and CD40;
(c) PLA2G4C, PRKCG, RAC2, PTPRC, FCGR3A, FCGR2A, SYK, and FCGR1A;
(d) PLA2G4C, PTPRC, RAC2, LAT, NCF1, FCGR3A, WAV1, SYK, PLA2G4B, WAS, PLCG2, HCK, FCGR2A, INPP5D, FCGR2A, INPP5D, FCGR1A, PIK3CD, PRKCD, and VASP;
(e) C1R, ITGAM, C4B, F13A1, ITGB2 and C1QC;
(f) SERPINA5, CR1, CR2, C4B, ITGAX, PROC, ITGAM, C3, F12, F13A1, ITGB2, SERPINE1, PLAU, CIS, C1QC, CFHR4, C1QA and C1R;
(g) HLA-DOB, KLRC2, CD8B, TAP1, HLA-DRA, CTSS, HLA-DMB, IFNG, CD8A and LGMN;
(h) HLA-DOB, KLRD1, TNF, IFNG, TAP1, HLA-G, CIITA, KLRC2, CD8B, TAP2, HLA-E, CD8A, CTSS, HLA-DPB1, HLA-DPA1, HLA-DRB1, HLA-DMB, HLA-DRA, HLA-DOA, HLA-F, CD74, HLA-DMA, IFI30, PSME1, HSP90AA1, and LGMN;
(i) CD79A, DAPP1, CD79B, CARD11, RAC2, BLNK, BTK, IFITIvII, and SYK; and
(j) CD79A, CD19, CARD11, CD79B, DAPP1, CR2, LILRA4, CD22, RAC2, BTK, LILRA1, BLNK, IFITM1, LILRB2, LILRB4, VAV1, SYK, PIK3AP1, PLCG2, INPP5D, CD72, IKBKG, IKBKB, PIK3CD, and CHUK;
(k) LCK, CD40LG, CD3D, CD28, CD3E, CARD11, PAK5, CD247, CD8B, ITK, PTPRC, LCP2, IFNG, CD8A, and FYN; and
(l) LCK, CD3D, CARD11, CD28, TNF, ZAP70, CD40LG, CD3E, CD3G, PTPRC, ITK, IFNG, ICOS, PDCD1, LAT, LCP2, GRAP2, IL10, CD8B, CD8A, CD247, VAV1, PAK5, IKBKG, IKBKB, PIK3CD, MAPK13, MAP3K7, and CHUK.

25. The method of claim 11, wherein the expression levels of the set of genes in the biological sample relative to the predetermined threshold levels indicates a likelihood AAV-induced inflammation in the retina, and wherein the set of genes is selected from:
(a) CXCL10, CXCL9, CCL5, CCL4L2, and MYD88;
(b) CXCL10, CXCL9, CCL5, CXCL11, IL1B, TLR8, CCL4L2, CD86, TLR2, STAT1, TNF, LY97, TLR4, IRF7, TLR7 and IKBKE;
(c) RAC2, SYK, VAV2, FCGR3A and PTPRC;
(d) RAC2, FCGR2A, PTPRC, FCGR3A, SYK, VAV1, NCF1, PLA2G4C, WAS, SCIN, FCGR1A, INPP5D, HCK and PLD1;
(e) ITGAX, FGG and CPB2;
(f) F13A1, ITGAX, C1QB, ITGAM, C1QC, C1QA, C2, ITGB2, C3, C4B, C1S, SERPINA1, VSIG4, SERPINA5, CR2, A2M, and F2RL3;
(g) HLA-DRB1, HLA-DQA1, HLA-DPA1, and KLRD1;
(h) HLA-DRA, HLA-G, B2M, HLA-DQA1, HLA-DRB1, HLA-DPB1, CIITA, CD74, HLA-DOA, HLA-DMB, HLA-DMA, HLA-E, CD8B, HLA-DPA1, CD4, TAP1, IFI30, KLRD1, HLA-DOB, TNF, CD8A, TAP2, and PSME1;
(i) DAPP1, RAC2, SYK, VAV2, and NFATC2;
(j) RAC2, CD79A, CD79B, DAPP1, LILRA4, SYK, VAV1, BTK, BLNK, LILRB2, INPP5D, NFATC2, LILRB4, CD22, CARD11, CR2, LILRA1, CD72 and RASGRP3
(k) VAV2, CD40LG, CD28, PTPRC, and NFATC2; and
(l) CD3D, PTPRC, CD28, LCK, CD3G, VAV1, ICOS, CD8B, CD4, CD40LG, ITK, CD3E, TNF, CD8A, NFATC2, LCP2, CARD11, and CTLA4.

26. A method of screening an rAAV composition for reduced AAV-induced inflammation comprising: (a) administering a dose of a test rAAV composition to a non-human subject; (b) obtaining a biological sample from the subject; (b) detecting expression levels of each of a set of genes in the biological sample; (d) comparing the detected expression levels for the test rAAV composition to expression levels detected for a dose of a control rAAV composition.

27. The method of claim 26, wherein the set of genes comprises at least two genes selected from A2M, B2M, BLNK, BTK, C1QA, C1QB, C1QC, C1R, C1S, C2, C3, C4B, CARD11, CASP8, CCL3, CCL4L2, CCL5, CD14, CD19, CD22, CD247, CD28, CD3D, CD3E, CD3G, CD4, CD40, CD40LG, CD72, CD74, CD79A, CD79B, CD86, CD8A, CD8B, CFHR4, CHUK, CIITA, CPB2, CR1, CR2, CTLA4, CTSS, CXCL10, CXCL11, CXCL8, CXCL9, CXCR3, DAPP1, F12, F13A1, F2RL3, FCGR1A, FCGR2A, FCGR3A, FYN, GRAP2, HCK, HLA-DMA, HLA-DMB, HLA-DOA, HLA-DOB, HLA-DPA1, HLA-DPB1, HLA-DQA1, HLA-DRA, HLA-DRB1, HLA-E, HLA-F, HLA-G, HSP90AA1, ICOS, IFI30, IFITIvII, IFITM1, IFNG, IKBKB, IKBKE, IKBKG, IL10, IL12A, IL12B, IL1B, IL6, INPP5D, IRF7, ITGAM, ITGAX, ITGB2, ITK, IY96, KLRC2, KLRD1, KPK3CD, LAT, LCK, LCP2, LGMN, LILRA1, LILRA4, LILRB2, LILRB4, LR2, LY97, MAP3K7, MAPK13, MP3K7, MYD88, NCF1, NFATC2, PAK5, PDCD1, PIK3AP1, PIK3CD, PLA2G4B, PLA2G4C, PLAU, PLCG2, PLD1, PRKCD, PROC, PSME1, PTPRC, RAC2, RASGRP3, SCIN, SERPINA1, SERPINA5, SERPINE1, STAT1, SYK, TAP1, TAP2, TLR1, TLR4, TLR7, TLR8, TLR10, TNF, VASP, VAV1, VAV2, VSIG4, WAS, WAV1, and ZAP70.

28. The method of any one of claims 26-27, wherein the set of genes is selected from:
(a) CXCL9, CXCL10, CXCL11, CCL5 and CCL3;
(b) CXCL9, CXCL11, CXCL10, IL6, TNF, CCL5, CXCL8, CCL3, TLR8, CCL4L2, IY96, IL12A, IL12B, STAT1, IKBKE, IL1B, TLR1, CASP8, IKBKG, TLR4, IRF7, CD14, MYD88, CD86, IKBKB, KPK3CD, MP3K7, CHUK, and CD40;
(c) CXCL10, CXCL9, CCL5, CCL4L2, and MYD88;
(d) CXCL10, CXCL9, CCL5, CXCL11, IL1B, TLR8, CCL4L2, CD86, TLR2, STAT1, TNF, LY97, TLR4, IRF7, TLR7 and IKBKE;
(e) PLA2G4C, PRKCG, RAC2, PTPRC, FCGR3A, FCGR2A, SYK, and FCGR1A;
(f) PLA2G4C, PTPRC, RAC2, LAT, NCF1, FCGR3A, WAV1, SYK, PLA2G4B, WAS, PLCG2, HCK, FCGR2A, INPP5D, FCGR2A, INPP5D, FCGR1A, PIK3CD, PRKCD, and VASP;
(g) RAC2, SYK, VAV2, FCGR3A and PTPRC;
(h) RAC2, FCGR2A, PTPRC, FCGR3A, SYK, VAV1, NCF1, PLA2G4C, WAS, SCIN, FCGR1A, INPP5D, HCK and PLD1;
(i) C1R, ITGAM, C4B, F13A1, ITGB2 and C1QC;
(j) SERPINA5, CR1, CR2, C4B, ITGAX, PROC, ITGAM, C3, F12, F13A1, ITGB2, SERPINE1, PLAU, C1S, C1QC, CFHR4, C1QA and C1R;
(k) ITGAX, FGG and CPB2;
(l) F13A1, ITGAX, C1QB, ITGAM, C1QC, C1QA, C2, ITGB2, C3, C4B, C1S, SERPINA1, VSIG4, SERPINA5, CR2, A2M, and F2RL3;
(m) HLA-DOB, KLRC2, CD8B, TAP1, HLA-DRA, CTSS, HLA-DMB, IFNG, CD8A and LGMN;
(n) HLA-DOB, KLRD1, TNF, IFNG, TAP1, HLA-G, CIITA, KLRC2, CD8B, TAP2, HLA-E, CD8A, CTSS, HLA-DPB1, HLA-DPA1, HLA-DRB1, HLA-DMB, HLA-DRA, HLA-DOA, HLA-F, CD74, HLA-DMA, IFI30, PSME1, HSP90AA1, and LGMN;
(o) HLA-DRB1, HLA-DQA1, HLA-DPA1, and KLRD1;
(p) HLA-DRA, HLA-G, B2M, HLA-DQA1, HLA-DRB1, HLA-DPB1, CIITA, CD74, HLA-DOA, HLA-DMB, HLA-DMA, HLA-E, CD8B, HLA-DPA1, CD4, TAP1, IFI30, KLRD1, HLA-DOB, TNF, CD8A, TAP2, and PSME1;
(q) CD79A, DAPP1, CD79B, CARD11, RAC2, BLNK, BTK, IFITIvII, and SYK;
(r) CD79A, CD19, CARD11, CD79B, DAPP1, CR2, LILRA4, CD22, RAC2, BTK, LILRA1, BLNK, IFITM1, LILRB2, LILRB4, VAV1, SYK, PIK3AP1, PLCG2, INPP5D, CD72, IKBKG, IKBKB, PIK3CD, and CHUK;
(s) DAPP1, RAC2, SYK, VAV2, and NFATC2; and
(t) RAC2, CD79A, CD79B, DAPP1, LILRA4, SYK, VAV1, BTK, BLNK, LILRB2, INPP5D, NFATC2, LILRB4, CD22, CARD11, CR2, LILRA1, CD72 and RASGRP3
(u) LCK, CD40LG, CD3D, CD28, CD3E, CARD11, PAK5, CD247, CD8B, ITK, PTPRC, LCP2, IFNG, CD8A, and FYN;
(v) LCK, CD3D, CARD11, CD28, TNF, ZAP70, CD40LG, CD3E, CD3G, PTPRC, ITK, IFNG, ICOS, PDCD1, LAT, LCP2, GRAP2, IL10, CD8B, CD8A, CD247, VAV1, PAK5, IKBKG, IKBKB, PIK3CD, MAPK13, MAP3K7, and CHUK;
(w) VAV2, CD40LG, CD28, PTPRC, and NFATC2; and
(x) CD3D, PTPRC, CD28, LCK, CD3G, VAV1, ICOS, CD8B, CD4, CD40LG, ITK, CD3E, TNF, CD8A, NFATC2, LCP2, CARD11, and CTLA4.

29. The method of any one of claims 26-28, wherein the test rAAV composition and control rAAV compositions are the same, and the dose of the test rAAV composition is different from the dose of the control rAAV composition.

30. The method of any one of claims 26-28, wherein the test rAAV composition and control rAAV compositions are different, and the dose of the test rAAV composition is the same as the dose of the control rAAV composition.
